(19) United States
(12) Patent Application Publication
(10) Pub. No.: US 2025/0134890 A1
(43) Pub. Date: May 1, 2025

(54) ANTIMALARIAL HEXAHYDROPYRIMIDINE ANALOGUES

(71) Applicant: UCB BIOPHARMA SRL, Brussels (BE)

(72) Inventors: Jeffrey BRUFFAERTS, Slough Berkshire (GB); Teresa DE HARO GARCIA, Brussels (BE); Martin Alexander LOWE, Slough Berkshire (GB); Komei SAKATA, Slough Berkshire (GB); Luigi Piero STASI, Slough Berkshire (GB); Richard David TAYLOR, Slough Berkshire (GB); Lewis Robin, Slough Berkshire (GB); Mengyang XUAN, Slough Berkshire (GB)

(21) Appl. No.: 18/835,890

(22) PCT Filed: Feb. 3, 2023
(86) PCT No.: PCT/EP2023/052674

§ 371 Date: Aug. 5, 2024

(30) Foreign Application Priority Data
Feb. 8, 2022 (GB) 2201613.3

(51) Int. Cl. A61K 31/513; A61K 31/675; A61P 33/06; C07D 239/22; C07D 405/04; C07D 405/14; C07F 9/6558

(52) U.S. Cl. CPC A61K 31/513; A61K 31/675; A61P 33/06; C07D 239/22; C07D 405/04; C07D 405/14; C07F 9/65583; C07F 9/65586

(57) ABSTRACT

A series of 2-imino-6-methylhexahydropyrimidin-4-one derivatives, substituted in the 6-position by an arylphenyl or heteroarylphenyl moiety, being potent inhibitors of the growth and propagation of the Plasmodium falciparum parasite in human blood, are beneficial as pharmaceutical agents, especially in the treatment of malaria.

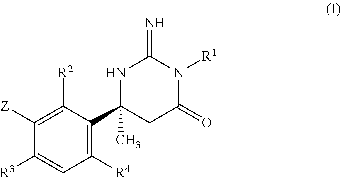

The present invention relates to a class of heterocyclic compounds, and to their use in therapy. More particularly, this invention is concerned with pharmacologically active substituted hexahydropyrimidine derivatives, and analogues thereof. These compounds are potent inhibitors of the growth and propagation of the Plasmodium falciparum parasite in human blood, and are accordingly of benefit as pharmaceutical agents, especially in the treatment of malaria.

Malaria is a mosquito-borne infectious disease, caused by a parasite of the genus Plasmodium, which has devastating consequences. Based on 2019 data, approximately 229 million cases, resulting in approximately 409,000 deaths, are reported annually. Approximately 80% of cases occur in sub-Saharan Africa, mostly in young children (aged 5 years or less).

The compounds in accordance with the present invention, being potent inhibitors of the growth and propagation of the P. falciparum parasite in human blood, are therefore beneficial in the treatment of malaria.

In addition, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2019/192992, WO 2020/229427, WO 2021/032687 and WO 2022/008639 describe certain classes of heterocyclic compounds which are stated to be potent inhibitors of the growth and propagation of the P. falciparum parasite in human blood, and therefore to be beneficial in the treatment of malaria.

WO 2017/142825 describes a family of heterocyclic compounds which are stated to be potent inhibitors of P. falciparum growth in vitro that may be useful for the treatment of malaria.

WO 2017/089453 and WO 2017/144517 describe heterocyclic compounds which are stated to be potent and selective inhibitors of plasmepsin V activity that are beneficial in the treatment of malaria.

WO 2016/172255 describes a class of heterocyclic compounds which are stated to be BACE inhibitors that may be useful for treating amyloid-o-protein-related (AP-related) pathologies including Alzheimer's disease.

WO 2008/103351, WO 2006/065277 and WO 2005/058311 describe a family of heterocyclic compounds that are stated to be aspartyl protease inhibitors. The compounds described in those publications are also stated to be effective in a method of inhibiting inter alia plasmepsins (specifically plasmepsins I and II) for treatment of malaria.

WO 2006/041404 describes a family of heterocyclic compounds that are stated to be inhibitors of Beta site APP (amyloid precursor protein) Cleaving Enzyme (BACE).

The compounds described in that publication are also stated to be effective in a method of modulating BACE activity; and in methods of treating or preventing an Aβ-related pathology, including Downs syndrome and Alzheimer disease.

The present invention provides a compound of formula (I), or a pharmaceutically acceptable salt thereof:

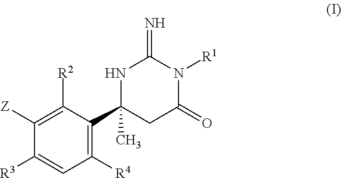

wherein

Z represents aryl or heteroaryl, either of which groups may be optionally substituted by one or more substituents;

R¹ represents C₃₋₇ cycloalkyl, C₃₋₇ cycloalkyl(C₁₋₆)alkyl, aryl(C₁₋₆)alkyl, C₃₋₇ heterocycloalkyl, C₃₋₇ heterocycloalkyl(C₁₋₆)alkyl, C₄₋₉ heterobicycloalkyl, C₄₋₉ spiroheterocycloalkyl or heteroaryl(C₁₋₆)alkyl, any of which groups may be optionally substituted by one or more substituents; and

R², R³ and R⁴ independently represent hydrogen, halogen or trifluoromethyl.

The present invention also provides a compound of formula (I) as defined above, or a pharmaceutically acceptable salt thereof, for use in therapy.

The present invention also provides a compound of formula (I) as defined above, or a pharmaceutically acceptable salt thereof, for use in the treatment and/or prevention of malaria.

The present invention also provides a method for the treatment and/or prevention of malaria which comprises administering to a patient in need of such treatment an effective amount of a compound of formula (I) as defined above, or a pharmaceutically acceptable salt thereof.

The present invention also provides the use of a compound of formula (I) as defined above, or a pharmaceutically acceptable salt thereof, for the manufacture of a medicament for the treatment and/or prevention of malaria.

Where any of the groups in the compounds of formula (I) above is stated to be optionally substituted, this group may be unsubstituted, or substituted by one or more substituents. Typically, such groups will be unsubstituted, or substituted by one, two or three substituents, generally by one or two substituents.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002.

Suitable alkyl groups which may be present on the compounds of use in the invention include straight-chained and branched C₇₋₆ alkyl groups, for example CI-4 alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulfonyl” and “C₁₋₆ alkylamino” are to be construed accordingly.

The term “C₃₋₇ cycloalkyl” as used herein refers to monovalent groups of 3 to 7 carbon atoms derived from a saturated monocyclic hydrocarbon, and may comprise benzo-fused analogues thereof. Suitable C₃₋₇ cycloalkyl groups include cyclopropyl, cyclobutyl, benzocyclobutenyl, cyclopentyl, indanyl, cyclohexyl and cycloheptyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, dioxanyl, tetrahydrothiopyranyl, piperidinyl, 1,2,3,4-tetrahydroquinolinyl, 1,2,3,4-tetrahydro-isoquinolinyl, piperazinyl, 1,2,3,4-tetrahydroquinoxalinyl, hexahydro-[1,2,5]thiadiazolo-[2,3-a]pyrazinyl, homopiperazinyl, morpholinyl, benzoxazinyl, thiomorpholinyl, azepanyl, oxazepanyl, diazepanyl, thiadiazepanyl and azocanyl.

The term “C₄₋₉ heterobicycloalkyl” as used herein refers to monovalent groups of 4 to 9 carbon atoms derived from a saturated bicyclic hydrocarbon, comprising one or more heteroatoms selected from oxygen, sulphur and nitrogen. Typical heterobicyclo-alkyl groups include 3-azabicyclo[3.1.0]hexanyl, 2-oxa-5-azabicyclo[2.2.1]heptanyl, 7-oxabicyclo[2.2.1]hexanyl, 6-azabicyclo[3.2.0]heptanyl, 3-azabicyclo[3.1.1]heptanyl, 6-oxa-3-azabicyclo[3.1.1]heptanyl, 3-azabicyclo[4.1.0]heptanyl, 2-oxabicyclo[2.2.2]-octanyl, quinuclidinyl, 2-oxa-5-azabicyclo[2.2.2]octanyl, 3-azabicyclo[3.2.1]octanyl, 8-oxabicyclo[3.2.1]octanyl, 8-azabicyclo[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo-[3.3.1]nonanyl, 3,7-dioxa-9-azabicyclo[3.3.1]nonanyl and 3,9-diazabicyclo[4.2.1]-nonanyl.

The term “C₄₋₉ spiroheterocycloalkyl” as used herein refers to saturated bicyclic ring systems containing 4 to 9 carbon atoms and at least one heteroatom selected from oxygen, sulphur and nitrogen, in which the two rings are linked by a common atom. Suitable spiroheterocycloalkyl groups include 5-azaspiro[2.3]hexanyl, 5-azaspiro[2.4]-heptanyl, 2-oxaspiro[3.3]heptanyl, 2-azaspiro[3.3]heptanyl, 2-oxa-6-azaspiro[3.3]-heptanyl, 3-oxa-6-azaspiro[3.3]heptanyl, 6-thia-2-azaspiro[3.3]heptanyl, 2-oxa-6-aza-spiro[3.4]octanyl, 2-oxa-6-azaspiro[3.5]nonanyl, 7-oxa-2-azaspiro[3.5]nonanyl, 2-oxa-7-azaspiro[3.5]nonanyl and 2,4,8-triazaspiro[4.5]decanyl.

The term “heteroaryl” as used herein refers to monovalent aromatic groups containing at least five atoms derived from a single ring or multiple condensed rings, wherein one or more carbon atoms have been replaced by one or more heteroatoms selected from oxygen, sulfur and nitrogen. Suitable heteroaryl groups include furyl, benzofuryl, dibenzofuryl, thienyl, benzothienyl, thieno[2,3-c]pyrazolyl, thieno[3,2-c]-pyridinyl, dibenzothienyl, pyrrolyl, indolyl, pyrrolo[2,3-b]pyridinyl, pyrrolo[3,2-c]-pyridinyl, pyrrolo[3,4-b]pyridinyl, pyrazolyl, pyrazolo[1,5-a]pyridinyl, pyrazolo[3,4-b]-pyridinyl, pyrazolo[3,4-d]pyrimidinyl, pyrazolo[1,5-a]pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]-pyridinyl, imidazo[1,5-a]pyridinyl, imidazo[4,5-b]pyridinyl, purinyl, imidazo[1,2-a]-pyrimidinvl, imidazo[1,2-a]pyrazinyl, oxadiazolyl, thiadiazolyl, triazolyl, [1,2,4]triazolo-[1,5-a]pyridinyl, [1,2,4]triazolo[1,5-a]pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl.

The term “halogen” as used herein is intended to include fluorine, chlorine, bromine and iodine atoms, typically fluorine, chlorine or bromine.

The absolute stereochemical configuration of the chiral carbon atom in the imino-tetrahydropyrimidinone nucleus of the compounds according to the invention is as depicted in formula (I) above. Generally, the compounds in accordance with the invention are at least 51% enantiomerically pure (by which it is meant that a sample thereof comprises a mixture of enantiomers containing 51% or more of the enantiomer depicted in formula (I) and 49% or less of the opposite antipode). Typically, the compounds in accordance with the invention are at least 60% enantiomerically pure. Appositely, the compounds in accordance with the invention are at least 75% enantiomerically pure. Suitably, the compounds in accordance with the invention are at least 80% enantiomerically pure. More suitably, the compounds in accordance with the invention are at least 85% enantiomerically pure. Still more suitably, the compounds in accordance with the invention are at least 90% enantiomerically pure. Even more suitably, the compounds in accordance with the invention are at least 95% enantiomerically pure. Preferably, the compounds in accordance with the invention are at least 99% enantiomerically pure. Ideally, the compounds in accordance with the invention are at least 99.9% enantiomerically pure.

Where the compounds of formula (I) have one or more additional asymmetric centres, they may accordingly exist as enantiomers. Where the compounds in accordance with the invention possess one or more additional asymmetric centres, they may also exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C=O)↔enol (CH═CHOH) tautomers or amide (NHC═O)↔hydroxyimine (N═COH) tautomers or imide (NHC═NH)↔aminoimine (N═CNH₂) tautomers. Formula (I) and the formulae depicted hereinafter are intended to represent all individual tautomers and all possible mixtures thereof, unless stated or shown otherwise. In addition, under certain circumstances, e.g. where R² represents halogen, compounds of formula (I) may exist as atropisomers. Formula (I) and the formulae depicted hereinafter are intended to represent all individual atropisomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium; D) or ³H (tritium; T) atom, preferably ¹H. Similarly, by way of example, each individual carbon atom present in formula (I), or in the formulae depicted hereinafter, may be present as a ¹²C, ¹³C or ⁴C atom, preferably ¹²C.

In a first embodiment, Z represents aryl, which group may be optionally substituted by one or more substituents. In a second embodiment, Z represents heteroaryl, which group may be optionally substituted by one or more substituents.

Typically, Z represents phenyl, naphthyl, furyl, benzofuryl, dibenzofuryl, thienyl, benzothienyl, thieno[2,3-c]pyrazolyl, thieno[3,2-c]pyridinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, pyrazolo[3,4-b]pyridinyl, pyrazolo[1,5-a]-pyrimidinyl, pyrazolo[3,4-d]pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, imidazo[1,5-a]-pyridinyl, imidazo[4,5-b]pyridinyl, purinyl, imidazo[1,2-a]pyrimidinyl, imidazo[1,2-a]-pyrazinyl, oxadiazolyl, thiadiazolyl, triazolyl, [1,2,4]triazolo[1,5-a]pyridinyl, [1,2,4]triazolo[4,3-a]pyridinyl, [1,2,4]triazolo[1,5-a]pyrimidinyl, benzotriazolyl, tetrazolyl, tetrazolo[1,5-a]pyridinyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl or chromenyl, any of which groups may be optionally substituted by one or more substituents.

Appositely, Z represents phenyl, naphthyl, pyrrolo[2,3-b]pyridinyl, pyrazolyl, pyrazolo[1,5-a]pyrimidinyl, indazolyl, isoxazolyl, benzimidazolyl, triazolyl, pyridinyl, pyridazinyl or pyrimidinyl, any of which groups may be optionally substituted by one or more substituents.

Suitably, Z represents phenyl or pyridinyl, either of which groups may be optionally substituted by one or more substituents.

Typical examples of optional substituents on Z include one, two or three substituents independently selected from halogen, cyano, nitro, C₁₋₆ alkyl, difluoromethyl, trifluoromethyl, trifluoroethyl, C₂₋₆ alkenyl, C₂₋₆ alkynyl, cyclopropyl, cyclobutyl, difluorocyclobutyl(C₁₋₆)alkyl, phenyl, fluorophenyl, trifluorophenyl, benzyl, phenyl-(C₂₋₆)alkenyl, morpholinyl, azepanyl, pyrrolidinyl(C₁₋₆)alkyl, 2-oxabicyclo[2.1.1]hexanyl-(C₁₋₆)alkyl, pyrazolyl, imidazolyl, (C₁₋₆)alkylimidazolyl, pyridinyl, pyridinyl(C₁₋₆)alkyl, hydroxy, hydroxy(C₁₋₆)alkyl, (difluoro)(hydroxy)(C₁₋₆)alkyl, (trifluoro)(hydroxy)(C₁₋₆)-alkyl, oxo, C₁₋₆ alkoxy, difluoromethoxy, difluoroethoxy, trifluoromethoxy, trifluoro-ethoxy, C₁₋₆ alkoxy(C₁₋₆)alkyl, phenoxy, methylenedioxy, difluoromethylenedioxy, C₁₋₆ alkylthio, C₁₋₆ alkylsulfinyl, C₁₋₆ alkylsulfonyl, amino, C₁₋₆ alkylamino, di(C₁₋₆)alkyl-amino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylaminosulfonyl, di(C₁₋₆)alkyl-sulfoximino and di(C₁₋₆)alkylphospboryl.

Apposite examples of optional substituents on Z include one, two or three substituents independently selected from halogen, cyano, C₁₋₆ alkyl, trifluoromethyl, trifluoroethyl, cyclopropyl, cyclobutyl, difluorocyclobutyl(C₁₋₆)alkyl, phenyl, fluoro-phenyl, trifluorophenyl, benzyl, phenyl(C₂₋₆)alkenyl, morpholinyl, azepanyl, pyrrolidinyl-(C₁₋₆)alkyl, 2-oxabicyclo[2.1.1]hexanyl(C₁₋₆)alkyl, pyrazolyl, pyridinyl, pyridinyl-(C₁₋₆)alkyl, (difluoro)(hydroxy)(C₁₋₆)alkyl, (trifluoro)(hydroxy)(C₁₋₆)alkyl, oxo, C₁₋₆ alkoxy, difluoromethoxy, trifluoromethoxy, C₁₋₆ alkoxy(C₁₋₆)alkyl, C₁₋₆ alkylsulfonyl, di(C₁₋₆)alkylamino(C₁₋₆)alkyl, C₁₋₆ alkylsulfonylamino, di(C₁₋₆)alkylsulfoximino and di(C₁₋₆)alkylphosphoryl.

Suitable examples of optional substituents on Z include one, two or three substituents independently selected from halogen.

Typical examples of particular substituents on Z include one, two or three substituents independently selected from fluoro, chloro, bromo, cyano, nitro, methyl, ethyl, n-propyl, isopropyl, n-butyl, tert-butyl, difluoromethyl, trifluoromethyl, trifluoro-ethyl, vinyl, allyl, propynyl, cyclopropyl, cyclobutyl, difluorocyclobutylmethyl, phenyl, fluorophenyl, trifluorophenyl, benzyl, phenylethenyl, morpholinyl, azepanyl, pyrrolidinylethyl, 2-oxabicyclo[2.1.1]hexanylethyl, pyrazolyl, imidazolyl, methyl-imidazolyl, pyridinyl, pyridinylmethyl, pyridinylethyl, hydroxy, hydroxymethyl, hydroxyethyl, (difluoro)(hydroxy)ethyl, (trifluoro)(hydroxy)ethyl, hydroxyisopropyl, oxo, methoxy, isopropoxy, difluoromethoxy, difluoroetboxy, trifluoromethoxy, trifluoro-ethoxy, methoxymethyl, methoxyethyl, phenoxy, methylenedioxy, difluoromethylene-dioxy, methylthio, methylsulfinyl, methylsulfonyl, amⁱno, methylamino, dimethylamino, aminomethyl, dimethylaminomethyl, dimethylaminoethyl, acetylamino, methoxy-carbonylamino, methylsulfonylamino, formyl, acetyl, carboxy, methoxycarbonyl, ethoxy-carbonyl, aminocarbonyl, methylaminocarbonyl, dimethylaminocarbonyl, aminosulfonyl, methylaminosulfonyl, dimethylaminosulfonyl, dimethylsulfoximino and dimethyl-phosphoryl.

Apposite examples of particular substituents on Z include one, two or three substituents independently selected from fluoro, chloro, cyano, methyl, isopropyl, n-butyl, trifluoromethyl, trifluoroethyl, cyclopropyl, cyclobutyl, difluorocyclobutylmethyl, phenyl, fluorophenyl, trifluorophenyl, benzyl, phenylethenyl, morpholinyl, azepanyl, pyrrolidinyletbyl, 2-oxabicyclo[2.1.1]hexanylethyl, pyrazolyl, pyridinyl, pyridinylmethyl, pyridinylethyl, (difluoro)(hydroxy)ethyl, (trifluoro)(hydroxy)ethyl, oxo, methoxy, difluoromethoxy, trifluoromethoxy, methoxyethyl, methylsulfonyl, dimethylaminoethyl, methylsulfonylamino, dimethylsulfoximino and dimethylphosphoryl.

Suitable examples of particular substituents on Z include one, two or three substituents independently selected from fluoro and chloro.

Typical values of Z include phenyl, fluorophenyl, chlorophenyl, cyanophenyl, methylphenyl, isopropylphenyl, tert-butylphenyl, trifluoromethylphenyl, propynylphenyl, morpholinylphenyl, pyrazolylphenyl, imidazolylphenyl, metbylimidazolylphenyl, (difluoro)(hydroxy)ethylphenyl, (trifluoro)(hydroxy)ethylphenyl, methoxyphenyl, isopropoxyphenyl, difluoromethoxyphenyl, trifluoromethoxyphenyl, phenoxyphenyl, methylenedioxyphenyl, difluoromethylenedioxyphenyl, methylsulfonylphenyl, methylsulfonylaminophenyl, methoxycarbonylphenyl, dimethylsulfoximinophenyl, dimethylphosphorylphenyl, difluorophenyl, (chloro)(fluoro)phenyl, (cyano)(fluoro)-phenyl, (fluoro)(methyl)phenyl, (fluoro)(trifluoromethyl)phenyl, (fluoro)(methoxy)-phenyl, (fluoro)(difluoromethoxy)phenyl, (fluoro)(trifluoromethoxy)phenyl, (fluoro)-(methylsulfonyl)phenyl, (dimethylsulfoxinino)(fluoro)phenyl, (dimethylphosphoryl)-(fluoro)phenyl, dichlorophenyl, (chloro)(cyano)phenyl, (chloro)(methyl)phenyl, (chloro)-(difluoromethoxy)phenyl, (chloro)(trifluoromethoxy)phenyl, (chloro)(methylsulfonyl)-phenyl, (cyano)(trifluoromethyl)phenyl, (cyano)(methoxy)phenyl, (cyano)(difluoro-methoxy)phenyl, dimethylphenyl, dimethoxyphenyl, (methoxy)(methylsulfonyl)phenyl, trifluorophenyl, (chloro)(difluoro)phenyl, naphthyl, methoxynaphthyl, cyanofuryl, fluorobenzofuryl, (cyano)(methyl)pyrrolyl, methylindolyl, methylpyrrolo[2,3-b]pyridinyl, pyrazolyl, methylpyrazolyl, trifluoroethylpyrazolyl, dimethylpyrazolyl, (methyl)-(trifluoromethyl)pyrazolyl, (dimethyl)(phenyl)pyrazolyl, pyrazolo[1,5-a]pyridinyl, fluoropyrazolo[1,5-a]pyridinyl, methylpyrazolo[3,4-b]pyridinyl, pyrazolo[1,5-a]-pyrimidinyl, methylindazolyl, isoxazolyl, methylisoxazolyl, cyclopropylisoxazolyl, methylimidazolyl, trifluoroethylbenzimidazolyl, imidazo[1,2-a]pyridinyl, methylimidazo-[1,2-a]pyridinyl, imidazo[1,5-a]pyridinyl, methylimidazo[4,5-b]pyridinyl, imidazo[1,2-a]pyrazinyl, methyloxadiazolyl, butyltriazolyl, trifluoroethyltriazolyl, cyclobutyltriazolyl, difluorocyclobutylmethyltriazolyl, phenyltriazolyl, fluorophenyltriazolyl, trifluorophenyl-triazolyl, benzyltriazolyl, phenylethenyltriazolyl, azepanyltriazolyl, pyrrolidinylethyl-triazolyl, 2-oxabicyclo[2.1.1]hexanylethyltriazolyl, pyridinyltriazolyl, pyridinylmethyl-triazolyl, pyridinylethyltriazolyl, methoxyethyltriazolyl, dimethylaminoethyltriazolyl, [1,2,4]triazolo[1,5-a]pyridinyl, [1,2,4]triazolo[4,3-a]pyridinyl, tetrazolo[1,5-a]pyridinyl, pyridinyl, fluoropyridinyl, chloropyridinyl, cyanopyridinyl, methylpyridinyl, ethyl-pyridinyl, tert-butylpyridinyl, difluoromethylpyridinyl, trifluoromethylpyridinyl, trifluoroethylpyridinyl, propynylpyridinyl, cyclopropylpyridinyl, morpholinylpyridinyl, methoxypyridinyl, difluoromethoxypyridinyl, trifluoromethoxypyridinyl, difluoroethoxy-pyridinyl, trifluoroethoxypyridinyl, dimethylaminopyridinyl, difluoropyridinyl, (fluoro)-(trifluoromethyl)pyridinyl, (fluoro)(methoxy)pyridinyl, (chloro)(methyl)pyridinyl, (chloro)(trifluoromethyl)pyridinyl, (cyano)(methyl)pyridinyl, (cyano)(difluoromethyl)-pyridinyl, (methyl)(trifluoromethyl)pyridinyl, (methyl)(oxo)pyridinyl, (methoxy)-(methyl)pyridinyl, (methoxy)(trifluoromethyl)-pyridinyl, (difluoromethoxy)(methyl)-pyridinyl, quinolinyl, cyanoquinolinyl, difluoromethoxyquinolinyl, isoquinolinyl, methylisoquinolinyl, difluoromethoxyisoquinolinyl, naphthyridinyl, pyridazinyl, methylpyridazinyl, trifluoromethylpyridazinyl, trifluoroethoxypyridazinyl, cinnolinyl, pyrimidinyl, chloropyrimidinyl, methylpyrimidinyl, tert-butylpyrimidinyl, trifluoro-methylpyrimidinyl, pyrazinyl, methylpyrazinyl, tert-butylpyrazinyl, methoxypyrazinyl, difluoromethoxypyrazinyl, dimethylaminopyrazinyl, quinoxalinyl and trifluoromethyl-quinoxalinyl.

Suitable values of Z include chlorophenyl, cyanophenyl, isopropylphenyl, trifluoromethylphenyl, morpholinylphenyl, pyrazolylphenyl, (difluoro)(hydroxy)ethyl-phenyl, (trifluoro)(hydroxy)ethylphenyl, methylsulfonylphenyl, methylsulfonylamino-phenyl, dimethylsulfoximinophenyl, dimethylphosphorylphenyl, difluorophenyl, (chloro)(fluoro)phenyl, (fluoro)(metbylsulfonyl)phenyl, (dimethylsulfoximino)(fluoro)-phenyl, (dimethylphosphoryl)(fluoro)phenyl, dichlorophenyl, (chloro)(trifluorometboxy)-phenyl, naphthyl, methylpyrrolo[2,3-b]pyridinyl, pyrazolyl, trifluoroethylpyrazolyl, dimethylpyrazolyl, pyrazolo[1,5-a]-pyrimidinyl, methylindazolyl, isoxazolyl, methyl-isoxazolyl, cyclopropylisoxazolyl, trifluoroethylbenzimidazolylbutyltriazolyl, trifluoro-ethyltriazolyl, cyclobutyltriazolyl, difluorocyclobutylmethyltriazolyl, phenyltriazolyl, fluorophenyltriazolyl, trifluorophenyltriazolyl, benzyltriazolyl, phenylethenyltriazolyl, azepanyltriazolyl, pyrrolidinylethyltriazolyl, 2-oxabicyclo[2.1.1]hexanylethyltriazolyl, pyridinyltriazolyl, pyridinylmethyltriazolyl, pyridinylethyltriazolyl, methoxyethyl-triazolyl, dimethylaminoethyltriazolyl, pyridinyl, chloropyridinyl, cyclopropylpyridinyl, methoxypyridinyl, difluoromethoxypyridinyl, trifluoromethoxypyridinyl, (methyl)(oxo)-pyridinyl, pyridazinyl and pyrimidinyl.

Typically, R¹ represents C₃₋₇ cycloalkyl, C₃₋₇ cycloalkyl(C₁₋₆)alkyl, aryl(C₁₋₆)alkyl, C₃₋₇ heterocycloalkyl, C₃₋₇ heterocycloalkyl(C₁₋₆)alkyl or heteroaryl(C₁₋₆)alkyl, any of which groups may be optionally substituted by one or more substituents.

Suitably, R¹ represents C₃₋₇ cycloalkyl, C₃₋₇ cycloalkyl(C₁₋₆)alkyl or C₃₋₇ heterocycloalkyl, any of which groups may be optionally substituted by one or more substituents.

More particularly, R¹ represents C₃₋₇ cycloalkyl or C₃₋₇ heterocycloalkyl, either of which groups may be optionally substituted by one or more substituents.

Typical examples of R¹ include cyclobutyl, cyclohexyl, cyclopropylethyl, tetrahydrofuranyl, tetrahydropyranyl, tetrabydrothiopyranyl, piperidinyl, oxetanylmethyl, tetrahydropyranyhnethyl, 7-oxabicyclo[2.2.1]heptanyl, 8-oxabicyclo[3.2.1]octanyl and 2-oxaspiro[3.3]heptanyl, any of which groups may be optionally substituted by one or more substituents.

Suitable examples of R¹ include cyclobutyl, cyclohexyl, cyclopropylethyl, tetrahydropyranyl, tetrahydrothiopyranyl and piperidinyl, any of which groups may be optionally substituted by one or more substituents.

Illustrative examples of R¹ include cyclohexyl and tetrahydropyranyl, either of which groups may be optionally substituted by one or more substituents.

Typical examples of optional substituents on R¹ include one, two or three substituents independently selected from halogen, cyano, nitro, C₁₋₆ alkyl, difluoromethyl, trifluoromethyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaninosulfonyl and di(C₁₋₆)alkyl-aminosulfonyl.

Suitable examples of optional substituents on R¹ include one, two or three substituents independently selected from halogen, C₁₋₆ alkyl, hydroxy, oxo, C₁₋₆ alkoxy and di(C₁₋₆)alkylamino.

Typical examples of particular substituents on R¹ include one, two or three substituents independently selected from fluoro, chloro, bromo, cyano, nitro, methyl, ethyl, isopropyl, difluoromethyl, trifluoromethyl, hydroxy, hydroxymethyl, hydroxyethyl, hydroxyisopropyl, oxo, methoxy, difluoromethoxy, trifluoromethoxy, methylthio, methylsulfinyl, methylsulfonyl, amino, methylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methyl-aminocarbonyl, dimethylaminocarbonyl, aminosulfonyl, methylaminosulfonyl and dimethylaminosulfonyl.

Suitable examples of particular substituents on R¹ include one, two or three substituents independently selected from fluoro, methyl, hydroxy, oxo, methoxy and dimethylamino.

Typical values of R¹ include dimethylaminocyclobutyl, (hydroxy)(methyl)-cyclohexyl, (difluoro)(methyl)cyclohexyl, (difluoro)(hydroxy)cyclohexyl, (difluoro)-(methoxy)cyclohexyl, hydroxycyclopropylethyl, methyltetrahydropyranyl, hydroxy-tetrahydropyranyl, (dioxo)(methyl)tetrahydrothiopyranyl, methylpiperidinyl and dimethylpiperidinyl.

Illustrative values of R¹ include (difluoro)(methyl)cyclohexyl and (difluoro)-(methoxy)cyclohexyl.

Generally, R², R³ and R⁴ independently represent hydrogen or halogen.

Generally, R² represents hydrogen or halogen.

In a first embodiment, R² represents hydrogen. In a second embodiment, R² represents halogen, especially fluoro or chloro. In one aspect of that embodiment, R² represents fluoro. In another aspect of that embodiment, R² represents chloro. In a third embodiment, R² represents trifluoromethyl.

Selected values of R² include hydrogen, fluoro and chloro.

Suitably, R² represents chloro.

Generally, R³ represents hydrogen or halogen, especially hydrogen.

In a first embodiment, R³ represents hydrogen. In a second embodiment, R³ represents halogen, especially fluoro or chloro. In one aspect of that embodiment, R¹ represents fluoro. In another aspect of that embodiment, R³ represents chloro. In a third embodiment, R³ represents trifluoromethyl.

Selected values of R³ include hydrogen, fluoro and chloro.

Suitably, R³ represents hydrogen or fluoro.

Generally, R⁴ represents hydrogen or halogen, especially hydrogen.

In a first embodiment, R⁴ represents hydrogen. In a second embodiment, R⁴ represents halogen, especially fluoro or chloro. In one aspect of that embodiment, R⁴ represents fluoro. In another aspect of that embodiment, R⁴ represents chloro. In a third embodiment, R⁴ represents trifluoromethyl.

Suitably, R² represents hydrogen or halogen; R³ represents hydrogen or halogen; and R⁴ represents hydrogen.

Appositely, R² represents halogen; R³ represents hydrogen or halogen; and R⁴ represents hydrogen.

Generally, R² represents hydrogen or halogen; and R³ and R⁴ both represent hydrogen.

More particularly, R² represents halogen; and R³ and R⁴ both represent hydrogen.

One sub-class of compounds according to the invention is represented by the compounds of formula (IIA), and pharmaceutically acceptable salts thereof:

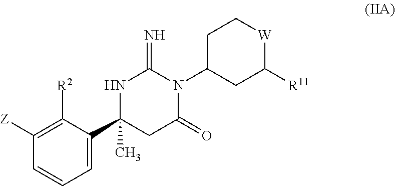

wherein

W represents O, S(O)₂, N—R¹², CH(OH) or CF₂;

R¹¹ represents hydrogen, methyl, hydroxy or methoxy;

R¹² represents hydrogen or methyl; and

Z and R² are as defined above.

In a first embodiment, W represents O. In a second embodiment, W represents S(O)₂. In a third embodiment, W represents N—R¹². In a fourth embodiment, W represents CH(OH). In a fifth embodiment, W represents CF₂.

Suitably, W represents O or CF₂.

In a first embodiment, R¹¹ represents hydrogen. In a second embodiment, R¹¹ represents methyl. In a third embodiment, R¹¹ represents hydroxy. In a fourth embodiment, R¹¹ represents methoxy.

Suitably, R¹¹ represents methyl or methoxy.

In a first embodiment, R¹² represents hydrogen. In a second embodiment, R¹² represents methyl.

Specific novel compounds in accordance with the present invention include each of the compounds whose preparation is described in the accompanying Examples, and pharmaceutically acceptable salts thereof.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

The compounds in accordance with the invention may be prepared by a process which comprises reacting a boronic acid derivative of formula Z—B(OH)₂ or a cyclic ester thereof formed with an organic diol, e.g. pinacol, 1,3-propanediol or neopentyl glycol, with a compound of formula (III):

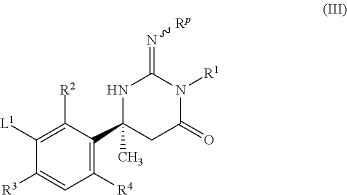

wherein Z, R¹, R², R³ and R⁴ are as defined above, L¹ represents a suitable leaving group, and R^{p} represents hydrogen or an N-protecting group; in the presence of a transition metal catalyst; followed, as necessary, by removal of the N-protecting group R^{p}.

The leaving group L¹ suitably represents halogen, e.g. bromo or iodo.

Suitably, the transition metal catalyst of use in the above process comprises [1,1′-bis(diphenylphosphino)ferrocene]dichloropalladium(II). Alternatively, the transition metal catalyst may comprise chloro(2-dicyclohexylphosphino-2′,4′,6′-triisopropyl-1,1′-biphenyl)[2-(2′-amino-1,1′-biphenyl)]palladium(II) (XPhos Pd G2) or (2-dicyclohexyl-phosphino-2′,4′,6′-triisopropyl-1,1′-biphenyl)[2-(2′-amino-1,1′-biphenyl)]palladium(II) methanesulfonate (XPhos Pd G3), either of which may conveniently be employed in conjunction with 2-dicyclohexylphosphino-2′,4′,6′-triisopropylbiphenyl (XPhos).

The reaction may generally be performed in the presence of a base, e.g. an inorganic base such as sodium carbonate, potassium carbonate or cesium carbonate, or potassium phosphate. The reaction will conveniently be effected at an elevated temperature in water and a suitable organic solvent, e.g. a cyclic ether such as 1,4-dioxane, or a C₁₋₄ alkanol such as ethanol.

Suitably, the N-protecting group R^{p} is tert-butoxycarbonyl (BOC).

Where the N-protecting group R^{p} is BOC, subsequent removal of the BOC group may suitably be accomplished by treatment with an acid, e.g. a mineral acid such as hydrochloric acid, or an organic acid such as trifluoroacetic acid. The reaction will typically be effected at ambient temperature in a suitable solvent, e.g. a chlorinated solvent such as dichloromethane, or a cyclic ether such as 1,4-dioxane.

The intermediates of formula (III) above wherein L¹ represents iodo may be prepared by reacting a compound of formula (IV):

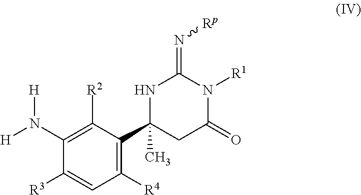

wherein R¹, R², R³, R⁴ and R^{p} are as defined above; with copper(I) iodide, in the presence of tert-butyl nitrite.

The reaction is conveniently accomplished at an elevated temperature in a suitable solvent, e.g. acetonitrile.

Alternatively, the intermediates of formula (III) above may be prepared by cyclising a compound of formula (V):

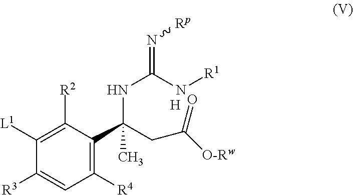

wherein R¹, R², R³, R⁴, L¹ and R^{p} are as defined above, and R^{w} represents C₁₋₄ alkyl.

Cyclisation of compound (V) is conveniently effected by treatment with a base.

Suitably, the base of use in the cyclisation reaction is a C₁₋₄ alkoxide salt, typically an alkali metal alkoxide such as potassium tert-butoxide. The reaction is conveniently accomplished at ambient temperature in a suitable solvent, e.g. a cyclic ether such as tetrahydrofuran.

The intermediates of formula (V) above may be prepared by reacting a compound of formula (VI) with a compound of formula (VII):

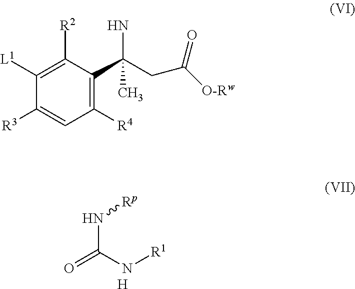

wherein R¹, R², R³, R⁴, L¹, R^{p} and R^{w} are as defined above.

Generally, the reaction between compounds (VI) and (VII) is performed in the presence of a coupling agent. A suitable coupling agent is N-(3-dimethylaminopropyl)-N′-ethylcarbodiimide hydrochloride (EDC·HCl). Suitably, the reaction is performed in the presence of a base, typically an organic base such as N,N-diisopropylethylamine.

The reaction between compounds (VI) and (VII) is conveniently accomplished at ambient temperature in a suitable solvent, e.g. a dipolar aprotic solvent such as N,N-dimethylformamide.

Under certain circumstances, the reaction between compounds (VI) and (VII) will proceed directly to the corresponding compound of formula (III).

The intermediates of formula (VI) above may be prepared from a compound of formula (VIII):

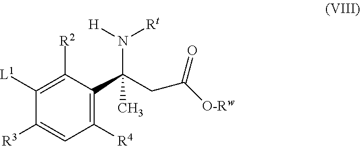

wherein R², R³, R⁴, L¹ and R^{w} are as defined above, and R^{t} represents an N-protecting group; by removal of the N-protecting group R^{t}.

Suitably, the N-protecting group R^{t} is tert-butoxycarbonyl (BOC).

Where the N-protecting group R^{t} is BOC, removal of the BOC group may suitably be accomplished by treatment with an acid, e.g. a mineral acid such as hydrochloric acid, or an organic acid such as trifluoroacetic acid.

The intermediates of formula (VIII) above wherein L¹ represents iodo may be prepared by reacting a compound of formula (IX):

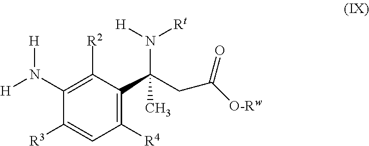

wherein R², R³, R⁴, R^{w} and R^{t} are as defined above; with copper(I) iodide, in the presence of tert-butyl nitrite, under conditions analogous to those described above for transforming compound (IV) to compound (III) wherein L¹ represents iodo.

The intermediates of formula (IX) above may be prepared by a process which comprises the following steps:
(i) attachment of the N-protecting group R^{t} to a compound of formula (X):

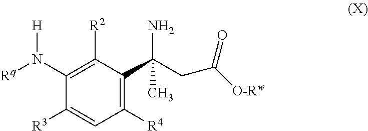

wherein R², R³, R⁴ and R⁷ are as defined above, and R^{q} represents an N-protecting group; and
(ii) removal of the N-protecting group R^{q} from the material thereby obtained.

Where the N-protecting group R^{t} represents BOC, step (i) may suitably be carried out by reacting compound (X) with di-tert-butyl dicarbonate. The reaction is generally effected in the presence of a mild base, e.g. an alkali metal bicarbonate such as sodium bicarbonate. The reaction is conveniently performed at ambient temperature in water and an organic solvent, e.g. a cyclic ether such as tetrahydrofuran.

Suitably, the N-protecting group R^{q} is benzyloxycarbonyl.

Where the N-protecting group R^{q} represents benzyloxycarbonyl, removal of the benzyloxycarbonyl group in step (ii) may suitably be accomplished by catalytic hydrogenation. Typically, this will involve treatment with gaseous hydrogen in the presence of a hydrogenation catalyst such as palladium on charcoal.

The intermediates of formulae (IV) and (X) above may be prepared by methods analogous to those described in WO 2021/032687.

In an alternative procedure, the compounds in accordance with the invention may be prepared by a process which comprises cyclising a compound of formula (XI):

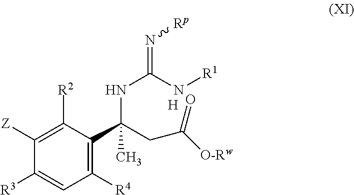

wherein Z, R¹, R², R³, R⁴, R^{p} and R^{w} are as defined above; under conditions analogous to those described above for the cyclisation of compound (V); followed, as necessary, by removal of the N-protecting group R^{p}, under conditions analogous to those described above.

The intermediates of formula (XI) above may be prepared by reacting a compound of formula (VII) as defined above with a compound of formula (XII):

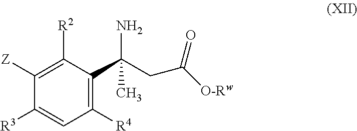

wherein Z, R², R³, R⁴ and R^{w} are as defined above; under conditions analogous to those described above for the reaction between compounds (VI) and (VII).

The intermediates of formula (XII) above may be prepared by reacting a boronic acid derivative of formula Z—B(OH)₂ or a cyclic ester thereof formed with an organic diol, e.g. pinacol, 1,3-propanediol or neopentyl glycol, with a compound of formula (VIII) as defined above, under conditions analogous to those described above for the reaction between compound (III) and a boronic acid derivative of formula Z—B(OH)₂ or a cyclic ester thereof formed with an organic diol; followed by removal of the N-protecting group R^{t}, under conditions analogous to those described above.

In another procedure, the compounds in accordance with the invention, wherein Z represents a 1,2,3-triazol-4-yl moiety substituted at the 1-position by a substituent Y, i.e. wherein Z represents a group of formula (Za):

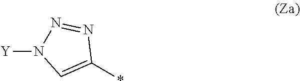

in which the asterisk (*) represents the point of attachment to the remainder of the molecule; may be prepared by a process which comprises reacting an azide derivative Y—N₃ with a compound of formula (XIII):

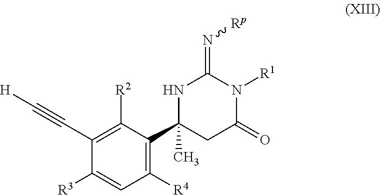

wherein R¹, R², R³, R⁴ and R^{p} are as defined above; followed, as necessary, by removal of the N-protecting group R^{p}, under conditions analogous to those described above.

The reaction between the azide derivative Y—N₃ and compound (XIII) is suitably performed in the presence of copper(II) sulphate and sodium ascorbate. The reaction may conveniently be effected at ambient or elevated temperature in water, optionally in the presence of an organic solvent, e.g. a C₁₋₄ alkanol such as tert-butanol.

The intermediates of formula (XIII) above may be prepared by a process which comprises the following steps:
(i) reaction of a compound of formula (III) as defined above with trimethylsilyl-acetylene; and
(ii) removal of the trimethylsilyl group from the material thereby obtained.
Step (i) will generally be accomplished in the presence of a transition metal catalyst. Suitably, the transition metal catalyst comprises bis(triphenylphosphine)-palladium(II) dichloride. The reaction may generally be performed in the presence of copper(I) iodide and a base, e.g. an organic amine such as triethylamine. The reaction will conveniently be effected at ambient temperature in a suitable organic solvent, e.g. a cyclic ether such as tetrahydrofuran.

Removal of the trimethylsilyl group in step (ii) may suitably be effected by treatment with a base, e.g. an alkali metal carbonate such as potassium bicarbonate. The reaction will conveniently be effected at ambient temperature in a suitable organic solvent, e.g. a C₁₋₄ alkanol such as methanol.

Where they are not commercially available, the starting materials of formula (VII) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N—BOC moiety (wherein BOC is an abbreviation for tert-butoxy-carbonyl) may be converted into the corresponding compound comprising a N—H moiety by treatment with an acid, e.g. a mineral acid such as hydrochloric acid, or an organic acid such as trifluoroacetic acid.

A compound comprising a tert-butyldimethylsilyloxy moiety may be converted into the corresponding compound comprising a hydroxy moiety by treatment with tetra-n-butylammonium fluoride.

A compound comprising a formyl (—CHO) moiety may be converted into the corresponding compound comprising a —CH(OH)CHF₂ functionality by treatment with (difluoromethyl)trimethylsilane in the presence of cesium fluoride; followed by treatment with tetra-n-butylammonium fluoride. Similarly, a compound comprising a formyl (—CHO) moiety may be converted into the corresponding compound comprising a —CH(OH)CF₃ functionality by treatment with (trifluoromethyl)trimethylsilane in the presence of cesium fluoride; followed by treatment with tetra-n-butylammonium fluoride.

A compound comprising a N—H functionality may be converted into the corresponding compound comprising a N—CH₃ functionality by treatment with formaldehyde in the presence of a reducing agent such as sodium cyanoborohydride.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The following Examples illustrate the preparation of compounds according to the invention.

The compounds of the present invention are potent inhibitors of the growth and propagation of the Plasmodium falciparum parasite in human blood. As such, they are active in a P. falciparum 3D7 asexual blood stage assay, exhibiting IC₅₀ values of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound).

### Asexual Blood Stage Assay

The assay used to measure the effect of test compounds on a bloodstream stage of Plasmodium falciparum: 3D7 strain employs SYBR green as the readout. This is a dye that binds to double stranded deoxyribonucleic acid (DNA) with a resulting increase in fluorescence, allowing detection of P. falciparum DNA in infected erythrocytes, and thereby providing a measure of parasite growth and propagation.

P. falciparum Culture Maintenance

Erythrocytes (A+blood) were prepared for both parasite culture and assay by washing 4 times with incomplete media (15.9 g RPMI 1640 (25 mM HEPES, L-glutamine), 1 g NaHCO₃, 2 g glucose, 400 μL gentacin (500 mg/mL), 2 mL hypoxanthine solution (13.6 g/L in 0.1M NaOH pH 7.3) in 1 litre of media). The cells were centrifuged at 1800 g for 5 minutes, before decanting the supernatant and re-suspending in fresh incomplete media. On the final wash, the cells were re-suspended in complete media (incomplete media with 5 g/L AlbumaxII), and centrifuged at 1800 g for 3 minutes. This cell sediment was treated as 100% haematocrit.

P. falciparum 3D7 was cultured in erythrocytes at 5% haematocrit in complete media at 37° C. (1% O₂, 3% CO₂, balance N₂). Cultures were split on a weekly basis to achieve a 1% parasitaemia in erythrocites at 5% haematocrit in fresh media. Culture media is replaced by fresh media every other day (2 times during the week).

### Assay Procedure

On day 1, test compounds were added to assay plates using Echo dispensing technology (1.5 fold dilution and 20 points titration). 50 nL of each compound dilution was added to 50 μL of culture (5% haematocrit, 0.5% parasitaemia) and incubated for 72 h at 37° C. (1% O₂, 3% CO₂, balance N₂). Final concentrations of test compounds ranged from 50,000 nM to 15 nM, in 0.5% DMSO.

On day 4, 10 μL SYBR green (Invitrogen S7563 supplied as 10,000×concentrate in DMSO) pre-diluted to 3×concentrate with Lysis buffer (20 mM Tris pH 7.9, 5% EDTA, 0.16% w/v, 1.6% TX100 v/v) was added to the cultures and incubated in the dark, overnight, at room temperature.

On day 5, fluorescent signal was measured using a BioTek plate reader (excitation 485 nm, emission 528 nm). All data were processed using IDBS ActivityBase. Raw data were converted into percent inhibition through linear regression by setting the high inhibition control (mefloquine) as 100% and the no inhibition control (DMSO) as 0%. Quality control criteria for passing plates were as follows: Z′>0.5, S:B>3, % CV_{(no inhibition control)}<15. The formula used to calculate Z′ is:

$$1 - \frac{3 ( σ p + σ n )}{( μ p + μn )}$$

where μ denotes the mean; σ denotes the standard deviation; p denotes the positive control; and n denotes the negative control.

All EC₅₀ curve fitting was undertaken using the following bi-phasic two site dose response using XLfit model 300 (IDBS):

$$y = \frac{A}{1 + 1 {0}^{( C - log 10 ( Bx ) )}} + \frac{100 - A}{1 + 1 {0}^{( D - log 10 ( Bx ) )}}$$

where A=100 minus the top of the upper curve 1 and the bottom of lower curve; B=Hill slope; log(C)=IC₅₀ concentration at lower site; log(D)=IC₅₀ concentration at upper site; x=inhibitor concentration; and y=% inhibition.

When tested in the P. falciparum 3D7 asexual blood stage assay as described above, the compounds of the accompanying Examples were found to exhibit the following IC₅₀ values.

| Example | IC₅₀ (nM) |
|---|---|
| 1 | 22 |
| 2 | 78 |
| 3 | 90 |
| 4 | 17 |
| 5 | 101 |
| 6 | 26 |
| 7 | 720 |
| 8 | 247 |
| 9 | 84 |
| 10 | 6 |
| 11 | 367 |
| 12 | 359 |
| 13 | 10 |
| 14 | 97 |
| 15 | 142 |
| 16 | 66 |
| 17 | 92 |
| 18 | 292 |
| 19 | 386 |
| 20 | 88 |
| 21 | 726 |
| 22 | 154 |
| 23 | 158 |
| 24 | 112 |
| 25 | 38 |
| 26 | 55 |
| 27 | 44 |
| 28 | 26 |
| 29 | 65 |
| 30 | 199 |
| 31 | 180 |
| 32 | 50 |
| 33 | 186 |
| 34 | 286 |
| 35 | 355 |
| 36 | 95 |
| 37 | 67 |
| 38 | 135 |
| 39 | 269 |
| 40 | 50 |
| 41 | 145 |
| 42 | 88 |
| 43 | 94 |
| 44 | 103 |
| 45 | 12 |
| 46 | 105 |
| 47 | 44 |
| 48 | 66 |
| 49 | 30 |
| 50 | 84 |
| 51 | 95 |
| 52 | 438 |
| 53 | 25 |
| 54 | 42 |
| 55 | 11 |
| 56 | 43 |
| 57 | 61 |
| 58 | 124 |
| 59 | 61 |
| 60 | 297 |
| 61 | 57 |
| 62 | 137 |
| 63 | 74 |
| 64 | 207 |
| 65 | 1304 |
| 66 | 272 |
| 67 | 88 |
| 68 | 1135 |
| 69 | 260 |
| 70 | 85 |
| 71 | 151 |
| 72 | 913 |
| 73 | 227 |
| 74 | 59 |
| 75 | 217 |
| 76 | 2460 |
| 77 | 460 |
| 78 | 365 |
| 79 | 107 |
| 80 | 1704 |
| 81 | 420 |
| 82 | 265 |
| 83 | 52 |
| 84 | 339 |
| 85 | 2674 |
| 86 | 286 |
| 87 | 2910 |
| 88 | 657 |
| 89 | 7953 |
| 90 | 122 |
| 91 | 6401 |
| 92 | 50 |
| 93 | 112 |
| 94 | 88 |
| 95 | 323 |
| 96 | 1429 |
| 97 | 280 |
| 98 | 297 |
| 99 | 1965 |
| 100 | 373 |
| 101 | 392 |
| 102 | 504 |
| 103 | 744 |
| 104 | 4937 |
| 105 | 1559 |
| 106 | 94 |
| 107 | 228 |
| 108 | 325 |
| 109 | 171 |
| 110 | 13 |
| 111 | 66 |
| 112 | 132 |
| 113 | 540 |
| 114 | 938 |
| 115 | 210 |
| 116 | 208 |
| 117 | 371 |
| 118 | 1008 |
| 119 | 296 |
| 120 | 507 |
| 121 | 185 |
| 122 | 101 |
| 123 | 542 |
| 124 | 216 |
| 125 | 220 |
| 126 | 5 |
| 127 | 100 |
| 128 | 112 |
| 129 | 11919 |

### EXAMPLES

Abbreviations

| | |
|---|---|
| DCM: dichloromethane | EtOAc: ethyl acetate |
| DMSO: dimethyl sulfoxide | THF: tetrahydrofuran |
| MeOH: methanol | DMF: N,N-dimethylformamide |
| DIPEA: N,N-diisopropylethylamine | TFA: trifluoroacetic acid |
| TFAA: trifluoroacetic anhydride | EtOH: ethanol |
| DME: 1,2-dimethoxyethane | DMAP: 4-(dimethylamino)pyridine |
| DAST: (diethylamino)sulfur trifluoride | TBAF: tetra-n-butylammonium fluoride |
| EDC•HCl: N-(3-dimethylaminopropyl)-N′-ethylcarbodiimide hydrochloride | |
| Xantphos: 4,5-bis(diphenylphosphino)-9,9-dimethylxanthene | |
| XPhos: 2-dicyclohexylphosphino-2′,4′,6′-triisopropylbiphenyl | |
| XPhos Pd G2: chloro(2-dicyclohexylphosphino-2′,4′,6′- | |
| triisopropyl-1,1′-biphenyl)[2-(2′- | |
| amino-1,1′-biphenyl)]palladium(II) | |
| XPhos Pd G3: (2-dicyclohexylphosphino-2′,4′,6′-triisopropyl- | |
| 1,1′-biphenyl)[2-(2′-amino- | |
| 1,1′-biphenyl)]palladium(II) methanesulfonate | |
| Pd₂(dba)₃: tris(dibenzylideneacetone)dipalladium(0) | |
| Pd(dppf)Cl₂: [1,1′- | |
| bis(diphenylphosphino)ferrocene]dichloropalladium(II) | |
| h: hour | M: mass |
| r.t.: room temperature | RT: retention time |
| DAD: Diode Array Detector | |
| HPLC: High Performance Liquid Chromatography | |
| UPLC: Ultra-Performance Liquid Chromatography | |
| LCMS: Liquid Chromatography Mass Spectrometry | |
| ESI: Electrospray Ionisation | |

### Nomenclature

Compounds were named in accordance with IUPAC guidelines with the aid of Biovia Draw version 20.1.

The asterisk (*)—for example, in compounds designated (2R*,4R*)-indicates compounds of known relative stereochemistry but unknown absolute stereochemistry.

### Analytical Conditions

### Method I

Stationary Phase: Waters X Bridge C18, 2.1×30 mm, 2.5 μm
Injection Volume: 5.0 μL
Flow Rate: 1.00 mL/minute
Detection: MS—ESI+m/z 150 to 800
   UV—DAD 220-400 nm
Mobile Phase A: 5 mM ammonium formate in water+0.1% ammonia
Mobile Phase B acetonitrile+5% Mobile Phase A+0.1% ammonia
Gradient program: 5% B to 95% B in 4.0 minutes; hold until 5.00 minutes;
   at 5.10 minutes concentration of B is 5%; hold up to 6.5 minutes

### Method 2

Stationary Phase: Phenomenex Gemini NX—C18 2×20 mm, 3 μM
Mobile Phase A: 10 mM ammonium formate in water+0.1% ammonia solution
Mobile Phase B: acetonitrile+5% water+0.1% ammonia solution
Flow Rate: 1 mL/minute

### Gradient Program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 3

Stationary Phase: Phenomenex Gemini NX—C18 2×20 mm, 3 μm
Mobile Phase A: 10 mM ammonium formate in water+0.1% ammonia solution
Mobile Phase B: acetonitrile+5% water+0.1% ammonia solution
Flow Rate: 1 mL/minute

### Gradient Program:

| Time | A % | B % |
|---|---|---|
| 0.00 | 95.00 | 5.00 |
| 4.00 | 5.00 | 95.00 |
| 5.00 | 5.00 | 95.00 |
| 5.10 | 95.00 | 5.00 |

### Method 4

Stationary Phase: Waters Acquity UPLC BEH C18 2.1×50 mm, 1.7 μm
Mobile Phase A: 10 mM ammonium formate in water+0.1% ammonia solution
Mobile Phase B: acetonitrile+5% water+0.1% ammonia solution
Flow rate: 1.5 mL/minute

### Gradient Program:

| Time | A % | B % |
|---|---|---|
| 0.00 | 95.00 | 5.00 |
| 0.10 | 95.00 | 5.00 |
| 3.50 | 5.00 | 95.00 |
| 4.00 | 5.00 | 95.00 |
| 4.05 | 95.00 | 5.00 |

### Method 5

Stationary Phase: Waters Acquity UPLC BEH C18 2.1×50 mm, 1.7 μm
Mobile Phase A: water/acetonitrile/ammonium formate (95:5:63 mg/L)+100 μg/L NH₄OH
Mobile Phase B: acetonitrile/water/ammonium formate (95:5:63 mg/L)+100 μg/L NH₄OH
Flow Rate: 0.8 mL/minute

### Gradient Program:

| Time | A % | B % |
|---|---|---|
| 0.00 | 99.00 | 1.00 |
| 0.15 | 99.00 | 1.00 |
| 1.60 | 5.00 | 95.00 |
| 1.65 | 5.00 | 95.00 |
| 2.00 | 5.00 | 95.00 |
| 2.05 | 99.00 | 1.00 |
| 2.75 | 99.00 | 1.00 |

### Method 6

Stationary Phase: Waters UPLC X Bridge BEH (C18, 2.1×50 mm, 2.5 μm)
Temperature: 45° C.
Injection Volume: 1.0 μL
Flow Rate: 1.00 mL/minute
Detection: Mass spectrometry—+/−detection in the same run
PDA: 210 to 400 nm
Mobile Phase A: 10 mM ammonium formate in water+0.1% formic acid
Mobile Phase B: 95% acetonitrile+5% H₂O+0.1% formic acid

### Gradient Program:

| Time | % A | % B |
|---|---|---|
| 0 | 95 | 55 |
| 0.10 | 95 | 5 |
| 2.10 | 5 | 95 |
| 2.35 | 5 | 95 |
| 2.80 | 95 | 5 |

### Method 7

LC-MS analysis performed on a Quattro micro API Tandem Quadrupole System Waters mass spectrometer. The spectrometer is equipped with a Waters 2720 LC separation module using a X-bridge C18 3.5 μm column in basic mode (water/acetonitrile/ammonium formate) and a Waters 996 (210 to 400 nm) photodiode array detector.

### Intermediate 1

### tert-Butyl (NE)-N-{(4S)-4-(2-chloro-3-iodophenyl)-4-methyl-1-[(2S,4S)-2-methyl-tetrahydropyran-4-yl]-6-oxohexahydropyrimidin-2-ylidene}carbamate

To a suspension of tert-butyl (NE)-N-{(4S)-4-(3-amino-2-chlorophenyl)-4-methyl-1-[(2S,4S)-2-methyltetrahydropyran-4-yl]-6-oxohexahydropyrimidin-2-ylidene}-carbamate (WO 2022/008639, Intermediate 9) (10.0 g, 22.2 mmol) in acetonitrile (60 mL) was added tert-butyl nitrite (90 mass %) (4.4 mL, 33.0 mmol) at 0° C., followed by CuI (8.50 g, 45.0 mmol). The reaction mixture was stirred at 60° C. for 5 h, then filtered. The filtrate was quenched with saturated aqueous NH₄Cl/NH₄OH solution (2:1, ca. 100 mL) and extracted with EtOAc (100 mL). The organic layer was separated, dried over anhydrous sodium sulfate and concentrated in vacuo. The crude residue was purified by flash chromatography (silica, 0-30% EtOAc in DCM) to afford the title compound (85% UV purity) (6.8 g, 46%) as a puffy white powder. LCMS (Method 2, ESI): 561.8 [MH]*, RT 2.97 minutes.

### Intermediate 2

### 6-Bromo-1-(2,2,2-trifluoroethyl)benzimidazole

To a solution of 6-bromo-1H-benzimidazole (700 mg, 3.45 mmol) in THF (15 mL) was added NaH (60 mass % in mineral oil) (210 mg, 5.25 mmol) at r.t. After 5 minutes, 2,2,2-trifluoroethyl trifluoromethanesulfonate (1.8 mL) was added. The reaction mixture was stirred at r.t. for 2 h, then carefully quenched with water (5 mL) and diluted with DCM (10 mL). The organic layer was separated and dried over anhydrous sodium sulfate, then concentrated in vacuo. The crude residue was purified by flash chromatography (silica, 30% EtOAc in hexane) to obtain the title compound (50 mass %: 1:1 mixture with regioisomeric 5-bromo-1-(2,2,2-trifluoroethyl)benzimidazole) (905 mg, 94%) as a yellow solid. LCMS (Method 3, ESI): 281.0 [MH]⁺, RT 0.95 minutes.

### Intermediate 3

### 6-(4,4,5,5-Tetramethyl-1,3,2-dioxaborolan-2-yl)-1-(2,2,2-trifluoroethyl)benzimidazole

A suspension of Intermediate 2 (50 mass %) (905 mg, 3.24 mmol), potassium acetate (1.26 g, 12.8 mmol), bis(pinacolato)diboron (2.20 g, 8.2 mmol) and bis(triphenyl-phosphine)palladium(II) dichloride (450 mg, 0.64 mmol) in 1,4-dioxane (10 mL) was stirred at 90° C. for 3 h. The reaction mixture was quenched with water (20 mL) and diluted with EtOAc (50 mL). The phases were separated, and the aqueous phase was washed with EtOAc (3×10 mL). The combined organic fractions were dried over anhydrous sodium sulfate and concentrated in vacuo. The residue was purified by flash chromatography (silica, 40% EtOAc in hexane) to obtain the title compound (45 mass %; 1:1 mixture with regioisomeric 5-(4,4,5,5-tetramethyl-1,3,2-dioxaborolan-2-yl)-1-(2,2,2-trifluoroethyl)benzimidazole) (1.10 g, 97%) as a brown solid. LCMS (Method 3, ESI): 327.2 [MH]+, RT 1.08 minutes.

### Intermediates 4 & 5

### tert-Butyl N-[rac-(1S,3S,4R)-4-hydroxy-3-methylcyclohexyl]carbamate (Intermediate 4)

### tert-Butyl N-[rac-(1S,3S,4S)-4-hydroxy-3-methylcyclohexyl]carbamate (Intermediate 5)

To a solution of tert-butyl N-(4-hydroxy-3-methylphenyl)carbamate (2.0 g, 8.96 mmol) in 2-propanol (40 mL) was added ruthenium on alumina (metal loading 5%) (452.7 mg, 0.22 mmol). The mixture was placed under a H₂ atmosphere (10 bar) in a stainless steel reactor at 120° C. for 110 h, then cooled to r.t. The mixture was filtered through a pad of diatomaceous earth and washed with 2-propanol. The solvent was removed in vacuo and the crude material was purified by flash chromatography (silica, 10-90% EtOAc in heptane) to obtain Intermediate 4 (235 mg, 11%). Other fractions were combined, concentrated and re-purified by flash chromatography (silica, 50% EtOAc in heptane with 0.1% diethylamine) to obtain Intermediate 5 (150 mg, 7%).

Intermediate 4: δ_{H} (400 MHz, CDCl₃) 4.41 (s, 1H), 3.75 (q, J 2.7 Hz, 1H), 3.45 (s, 1H), 1.88 (dq, J 13.8, 3.3 Hz, 1H), 1.80-1.49 (m, 4H), 1.44 (m, 11H), 1.21 (q, J 11.8 Hz, 1H), 0.97 (d, J 6.8 Hz, 3H). LCMS (Method 7): 230 [MH]⁺, RT 5.24 minutes.

Intermediate 5: δ_{H} (400 MHz, CDCl₃) 4.34 (s, 1H), 3.47 (s, 1H), 3.12 (td, J 10.4, 4.1 Hz, 1H), 1.99 (m, J 16.2, 7.1, 3.4 Hz, 4H), 1.77-1.30 (m, 11H), 1.29-1.06 (m, 1H), 1.02 (d, J 6.5 Hz, 3H), 0.88 (q, J 12.2 Hz, 1H). LCMS (Method 7): 230 [MH]⁺, RT 5.10 minutes.

### Intermediate 6

### rac-(1R,2S,4S)-4-Amino-2-methylcyclohexanol hydrochloride

Intermediate 4 (500.0 mg, 2.18 mmol) was dissolved in 1,4-dioxane (5 mL), then 4N HCl in 1,4-dioxane (5 mL, 20 mmol) was added at r.t. The mixture was stirred for 7 h, then concentrated in vacuo. The crude material was freeze-dried to afford the title compound (474 mg, 2.86 mmol) as a clear oil, which was utilised without further purification. 6H (300 MHz, DMSO-d₆) 7.99 (s, 3H), 3.65-3.44 (m, 2H), 2.95 (d, J 4.9 Hz, 1H), 1.74 (m, 1H), 1.68-1.18 (m, 6H), 0.88 (d, J 6.2 Hz, 3H).

### Intermediate 7

### tert-Butyl N-{[rac-(1R,3R,4S)-4-hydroxy-3-methylcyclohexyl]carbamothioyl}carbamate

Prepared from Intermediate 6 (474.0 mg, 2.86 mmol) in accordance with the procedure described in WO 2022/008639 (for Intermediate 2 thereof) to afford the title compound (460.0 mg, 56%) as an off-white solid. LCMS (Method 3, ESI): 287.2 [MH]⁻, RT 1.70 minutes.

### Intermediate 8

### tert-Butyl N-({rac-(1R,3R,4S)-4-[tert-butyl(dimethyl)silyl]oxy-3-methylcyclohexyl}-carbamothioyl)carbamate

To a solution of Intermediate 7 in DMF (3 mL) were added imidazole (548 mg, 8.0 mmol) and tert-butyldimethylchlorosilane (371.0 mg, 2.4 mmol) at 0° C. The mixture was stirred at r.t. for 20 h, then additional tert-butyldimethylchlorosilane (371 mg, 2.39 mmol) was added. The mixture was stirred at 40° C. for 3 h, then quenched with water (50 mL) and extracted with EtOAc (2×50 mL). The organic layer was separated, dried over anhydrous sodium sulfate and concentrated in vacuo. The crude residue was purified by flash chromatography (silica, 0-20% EtOAc in hexane) to afford the title compound (450 mg, 70%) as a colourless oil. LCMS (Method 3, ESI): 403.2 [MH]⁺, RT 3.81 minutes.

### Intermediate 9

### tert-Butyl (NE)-N-[(4S)-4-[3-(benzyloxycarbonylamino)-2-chlorophenyl]-1-{(1SR,3SR,4RS)-4-[tert-butyl(dimethyl)silyl]oxy-3-methylcyclohexyl}-4-methyl-6-oxo-hexahydropyrimidin-2-ylidene]carbamate

Prepared from Intermediate 8 (450.0 mg, 1.12 mmol) in accordance with the procedure described in WO 2022/008639 (for Intermediate 8 thereof) to afford the title compound (500 mg, 62%) as an off-white solid. LCMS (Method 3, ESI): 713.2 [MH]⁺, RT 4.08 minutes.

### Intermediate 10

### tert-Butyl (NE)-N-[(4S)-4-(3-amino-2-chlorophenyl)-1-{(1SR,3SR,4RS)-4-[tert-butyl-(dimethyl)silyl]oxy-3-methylcyclohexyl}-4-methyl-6-oxohexahydropyrimidin-2-ylidene]carbamate

Prepared from Intermediate 9 (500 mg, 0.70 mmol) in accordance with the procedure described in WO 2022/008639 (for Intermediate 9 thereof) to afford the title compound (430 mg, 100%). LCMS (Method 3, ESI): 579.2 [MH]⁺, RT 3.74 minutes.

### Intermediate 11

### tert-Butyl (NE)-N-[(4S)-1-{(1SR,3SR,4RS)-4-[tert-butyl(dimethyl)silyl]oxy-3-methyl-cyclohexyl}-4-(2-chloro-3-iodophenyl)-4-methyl-6-oxohexahydropyrimidin-2-ylidene]-carbamate

Prepared from Intermediate 10 (430.0 mg, 0.74 mmol) in accordance with the procedure described for Intermediate 1 to afford the title compound (180.0 mg, 31%) as a colourless oil. LCMS (Method 3, ESI): 690.2 [MH]⁺, RT 4.17 minutes.

### Intermediate 12

### (8-Oxo-1,4-dioxaspiro[4.5]decan-7-yl) acetate

A mixture of 1,4-dioxaspiro[4.5]decan-8-one (7.50 g, 48.0 mmol) and lead(IV) acetate (31.9 g, 72.0 mmol) in toluene (150 mL) was heated at 110° C. for 16 h, after which time the reaction mixture was cooled to r.t., filtered through a pad of Celite® and washed with EtOAc (3×150 mL). The organic layer was separated, then dried over anhydrous Na₂SO₄ and concentrated in vacuo. The crude residue was purified by column chromatography (silica, 100-200 mesh, 20% EtOAc in hexanes) to afford the title compound (6.56 g, 64%) as an off-white solid. δ_{H} (400 MHz, DMSO-d₆) 5.24 (dd, J 13.2, 6.8 Hz, 1H), 3.89-4.08 (m, 4H), 2.54-2.69 (m, 1H), 2.23-2.33 (m, 2H), 2.07 (s, 3H), 1.93-2.01 (m, 2H). One proton signal merged with solvent peak.

### Intermediate 13

### (8,8-Difluoro-1,4-dioxaspiro[4.5]decan-7-yl) acetate

To a solution of Intermediate 12 (13.5 g, 63.0 mmol) in dry DCM (270 mL) was added DAST (20.8 mL, 158 mmol) at 0° C. The reaction mixture was stirred at 0° C. for 30 minutes, then heated at 50° C. for 5 h, after which time the reaction mixture was quenched with saturated aqueous NaHCO₃ solution (450 mL) and extracted with DCM (3×400 mE). The organic layer was separated, then dried over anhydrous Na₂SO₄ and concentrated in vacuo. The crude residue was purified by column chromatography (silica, 100-200 mesh, 15% EtOAc in hexanes) to afford the title compound (9.51 g, 64%) as a colourless oil. δ_{H} (400 MHz, DMSO-d₆) 5.18-5.30 (m, 1H), 3.85-3.99 (m, 4H), 2.10-2.25 (m, 1H), 2.08 (s, 3H), 1.98-2.06 (m, 2H), 1.87-1.98 (m, 1H), 1.72-1.81 (m, 2H).

### Intermediate 14

### 4,4-Difluoro-3-hydroxycyclohexanone

To a solution of Intermediate 13 (9.50 g, 40.2 mmol) in THF (95 mL) was added 10% aqueous HCl (380 mL) slowly at r.t. The reaction mixture was stirred at r.t. for 16 h, then quenched with saturated aqueous NaHCO₃ solution (450 mL) and extracted with DCM (3×450 mL). The organic layer was separated, washed with saturated aqueous NH₄Cl solution (200 mL) and brine (200 mL), then dried over anhydrous Na₂SO₄ and concentrated in vacuo, to afford the crude title compound (4.50 g) as a colourless oil, which was utilised without further purification. δ_{H} (400 MHz, CDCl₃) 4.22-4.33 (m, 1H), 3.74 (br s, 1H), 2.77-2.88 (m, 1H), 2.45-2.68 (m, 4H), 2.18-2.32 (m, 1H).

### Intermediate 15

### 3-[tert-Butyl(dimethyl)silyl]oxy-4,4-difluorocyclohexanone

To a solution of Intermediate 14 (4.50 g, 30.0 mmol) in dry DCM (90 mL) was added imidazole (5.10 g, 74.9 mmol). The reaction mixture was cooled to 0° C., then tert-butyldimethylsilyl chloride (11.3 g, 74.9 mmol) was added. The reaction mixture was stirred at r.t. for 16 h, then quenched with 10% aqueous NH₄Cl solution (150 mL) and extracted with EtOAc (3×150 mL). The organic layer was separated, then dried over anhydrous Na₂SO₄ and concentrated in vacuo. The crude residue was purified by column chromatography (silica, 100-200 mesh, 30% EtOAc in hexanes) to afford the title compound (7.00 g, 88%) as a colourless oil. 11 (400 MHz, CDCl₃) 4.18-4.27 (m, 1H), 2.80 (d, J 14.6 Hz, 1H), 2.34-2.60 (m, 4H), 2.08-2.28 (m, 1H), 0.89 (s, 9H), 0.11 (s, 6H).

### Intermediate 16

### rac-(1R,3R)—N-Benzyl-3-[tert-butyl(dimethyl)silyl]oxy-4,4-difluorocyclohexanamine

To a solution of Intermediate 15 (7.00 g, 26.5 mmol) in isopropanol (70 mL) were added benzylamine (4.34 mL, 39.7 mmol) and acetic acid (0.50 mL) under a nitrogen atmosphere. The reaction mixture was stirred at r.t. for 8 h, then NaBH₄ (2.50 g, 39.7 mmol) was added. The reaction mixture was stirred at r.t. for 16 h, then quenched with cold water (500 mL) and extracted with EtOAc (3×150 mL). The organic layer was separated, then dried over anhydrous Na₂SO₄ and concentrated in vacuo. The crude residue was purified by column chromatography (silica, 100-200 mesh, 30% EtOAc in hexanes) to afford the title compound (8.00 g, 71%) as a pale yellow oil. δ_{H} (400 MHz, CDCl₃) 7.32-7.44 (m, 5H), 3.87 (s, 2H), 3.74-3.83 (m, 1H), 2.76-2.86 (m, 2H), 2.08-2.25 (m, 2H), 1.98-2.07 (m, 1H), 1.61-1.96 (m, 3H), 0.88 (s, 9H), 0.09 (s, 6H). LCMS (Method 1, ESI): 356.10 [MH]⁺, RT 2.57 minutes.

### Intermediate 17

### rac-(1R,5R)-5-(Benzylamino)-2,2-difluorocyclohexanol

To a solution of Intermediate 16 (4.00 g, 9.39 mmol) in THF (60 mL) was added TBAF (1M in THF, 14.1 mL) under a nitrogen atmosphere at 0° C. The reaction mixture was stirred at r.t. for 2 h, then quenched with water (200 mL) and extracted with EtOAc (3×300 mL). The organic layer was separated, then dried over anhydrous Na₂SO₄ and concentrated in vacuo. The crude residue was purified by combi-flash chromatography (40% EtOAc in hexanes) to afford the title compound (1.90 g, 84%) as a colourless oil. δ_{H} (400 MHz, DMSO-d₆) 1.22-1.44 (m, 2H), 1.59-1.85 (m, 2H), 1.88-2.07 (m, 2H), 7.30 (q, J 7.66 Hz, 4H), 7.13-7.24 (m, 1H), 5.48-5.62 (m, 1H), 3.69 (s, 2H), 3.53-3.66 (m, 1H), 2.53-2.66 (m, 1H). One exchangeable proton signal not observed in NMR spectrum.

### Intermediate 18

### tert-Butyl N-benzyl-N-[rac-(1R,3R)-4,4-difluoro-3-hydroxycyclohexyl]carbamate

To a solution of Intermediate 17 (1.90 g, 7.87 mmol) in DCM (20 mL) was added triethylamine (2.20 mL, 15.7 mol). The reaction mixture was stirred at r.t. for 15 minutes, then di-tert-butyl dicarbonate (2.06 g, 9.45 mmol) was added at 0° C. The reaction mixture was stirred at r.t. for 16 h, then diluted with DCM (150 mL) and washed with water (100 mL). The organic layer was separated, then dried over anhydrous Na₂SO₄ and concentrated in vacuo. The crude residue was purified by combi-flash chromatography (20% EtOAc in hexanes) to afford the title compound (1.80 g, 67%) as an off-white solid. δ_{H} (400 MHz, DMSO-d₆) 7.28-7.37 (m, 2H), 7.17-7.27 (m, 3H), 5.37 (d, J 5.38 Hz, 1H), 4.34 (s, 2H), 3.92-4.07 (m, 1H), 3.55-3.75 (m, 1H), 1.89-2.02 (m, 1H), 1.40-1.84 (m, 5H), 1.34 (s, 9H). LCMS (Method 1, ESI): 286.00 {MH]⁺, RT 2.11 minutes.

### Intermediate 19

### tert-Butyl N-benzyl-N-[rac-(1R,3R)-4,4-difluoro-3-methoxycyclohexyl]carbamate

To a solution of Intermediate 18 (1.50 g, 4.38 mmol) in THF (30 mL) was added NaH (0.26 g, 6.57 mmol) at 0° C. under a nitrogen atmosphere. The reaction mixture was stirred at r.t. for 30 minutes, then iodomethane (0.93 g, 6.57 mmol) was added. The reaction mixture was stirred at r.t. for 2 h, then poured into water (160 mL) and extracted with EtOAc (2×160 mL). The organic layer was separated, then dried over anhydrous Na₂SO₄ and concentrated in vacuo, to afford the crude title compound (1.72 g) as a pale yellow semi-solid, which was utilised without further purification. δ_{H} (400 MHz, CDCl₃) 7.13-7.37 (m, 5H), 4.20-4.50 (m, 3H), 3.50 (s, 3H), 3.25-3.45 (m, 1H), 2.02-2.19 (m, 2H), 1.64-1.80 (m, 3H), 1.40 (s, 9H), 0.80-0.96 (m, 1H).

### Intermediate 20

### rac-(1R,3R)—N-Benzyl-4,4-difluoro-3-methoxycyclohexanamine

To a solution of Intermediate 19 (1.70 g, 4.78 mmol) in DCM (20 mL) was added TFA (1.10 mL, 14.3 mmol) at 0° C. The reaction mixture was stirred at r.t. for 2 h, then poured into cold saturated aqueous NaHCO₃ solution (80 mL) and extracted with EtOAc (3×80 mL). The organic layer was separated, then dried over anhydrous Na₂SO₄ and concentrated in vacuo. The crude residue was purified by combi-flash chromatography (40% EtOAc in hexanes) to afford the title compound (1.10 g, 90%) as a colourless oil. δ_{H} (400 MHz, DMSO-d₆) 7.25-7.38 (m, 4H), 7.16-7.24 (m, 1H), 3.71 (s, 2H), 3.40-3.53 (m, 1H), 3.37 (s, 3H), 2.55-2.63 (m, 1H), 2.10-2.26 (m, 2H), 1.92-2.05 (m, 1H), 1.82-1.92 (m, 1H), 1.60-1.80 (m, 1H), 1.10-1.26 (m, 2H).

### Intermediate 21

### rac-(1R,3R)-4,4-Difluoro-3-methoxy-cyclohexanamine

To a solution of Intermediate 20 (1.10 g, 4.31 mmol) in MeOH (22 mL) was added 20% Pd/C (0.23 g, 0.43 mmol) under a nitrogen atmosphere. The reaction mixture was stirred at r.t. for 2 h under hydrogen pressure, then filtered through a pad of Celite®, washing through with MeOH (3×30 mL). The filtrate was concentrated in vacuo to afford the title compound (0.67 g, 94%) as a colourless oil, which was utilised without further purification. δ_{H} (400 MHz, DMSO-d₆) 3.40-3.53 (m, 1H), 2.73 (t, J 10.76 Hz, 1H), 1.88-2.08 (m, 2H), 1.64-1.85 (m, 2H), 1.07-1.26 (m, 2H). Three proton signals merged into solvent peak; and two exchangeable proton signals not observed in NMR spectrum.

### Intermediate 22

### tert-Butyl N-{[rac-(1R,3R)-4,4-difluoro-3-methoxycyclohexyl]carbamothioyl}carbamate

To a solution of N,N′-bis-tert-butoxycarbonylthiourea (1.09 g, 3.94 mmol) in THF (8 mL) was added NaH (0.47 g, 11.8 mmol) at 0° C. The reaction mixture was stirred at 0° C. for 1 h, then TFAA (0.83 mL, 5.90 mmol) was added at 0° C. The reaction mixture was stirred at 0° C. for 1 h, then a solution of Intermediate 21 (0.65 g, 3.94 mmol) in THF (2 mL) was added at 0° C. The reaction mixture was stirred at r.t. for 16 h, then diluted with water (70 mL) and extracted with EtOAc (2×70 mL). The organic layer was separated and washed with brine (70 mL), then dried over anhydrous Na₂SO₄ and concentrated in vacuo. The crude residue was purified by column chromatography (silica, 100-200 mesh, 20% EtOAc in hexanes) to afford the title compound (1.10 g, 85%) as a pale yellow solid. δ_{H} (400 MHz, DMSO-d₆) 10.58 (s, 1H), 10.20 (d, J 6.85 Hz, 1H), 4.40-4.52 (m, 1H), 3.59-3.70 (m, 1H), 3.40 (s, 3H), 1.70-2.15 (m, 6H), 1.44 (s, 9H). LCMS (Method 1, ESI): 325.10 [MH]⁺, RT 1.98 minutes.

### Intermediate 23

### Methyl (3S)-3-[3-(benzyloxycarbonylamino)-2-chlorophenyl]-3-({(Z)—N′-tert-butoxy-carbonyl-N-[rac-(1R,3R)-4,4-difluoro-3-methoxycyclohexyl]carbamimidoyl}amino)-butanoate

To a solution of Intermediate 22 (1.10 g, 3.36 mmol) and methyl (3S)-3-amino-3-[3-(benzyloxycarbonylamino)-2-chlorophenyl]butanoate (WO 2022/008639, Intermediate 7) (1.39 g, 3.36 mmol) in DMF (10 mL) were added EDC·HCl (0.96 g, 5.03 mol) and DIPEA (1.17 mL, 6.71 mmol) at 0° C. The reaction mixture was stirred at r.t. for 16 h, then quenched with water (140 mL) and extracted with EtOAc (3×70 mL). The organic layer was separated, then dried over anhydrous Na₂SO₄ and concentrated in vacuo, to afford the crude title compound (2.80 g) as a pale yellow solid, which was utilised without further purification. LCMS (Method 1, ESI): 667.10 [MH]⁺, RT 2.35 minutes.

### Intermediate 24

### tert-Butyl (NE)-N-{(4S)-4-[3-(benzyloxycarbonylamino)-2-chlorophenyl]-1-[(1RS,3RS)-4,4-difluoro-3-methoxycyclohexyl]-4-methyl-6-oxohexahydropyrimidin-2-ylidene}-carbamate

To a solution of Intermediate 23 (2.80 g, 3.08 mol) in dry THF (28 mL) was added potassium tert-butoxide (1M in THF, 3.08 mL) at 0° C. The reaction mixture was stirred at 0° C. for 1.5 h, then quenched with water (150 mL) and extracted with EtOAc (3×80 mL). The organic layer was separated, then dried over anhydrous Na₂SO₄ and concentrated in vacuo. The residue was purified by combi-flash chromatography (25% EtOAc in hexanes) to afford the title compound (1.88 g, 94%) as an off-white solid. δ_{H} (400 MHz, DMSO-d₆) 10.51 (d, J 2.45 Hz, 1H), 9.24 (s, 1H), 7.58 (d, J 7.82 Hz, 1H), 7.29-7.45 (m, 6H), 7.18 (d, J 4.40 Hz, 1H), 5.14 (s, 2H), 4.45-4.63 (m, lH), 3.59 (d, J 16.63 Hz, 1H), 3.36-3.53 (m, 1H), 3.26-3.35 (s, 3H), 2.16-2.43 (m, 2H), 1.83-2.05 (m, 2H), 1.76 (s, 3H), 1.56-1.70 (m, 1H), 1.45 (s, 9H), 1.26-1.35 (m, 1H), 0.95-1.07 (m, 1H). LCMS (Method 1, ESI): 635.05 [MH]⁺, RT 2.36 minutes.

### Intermediate 25

### tert-Butyl (NE)-N-{(4S)-4-(3-amino-2-chlorophenyl)-1-[(1RS,3RS)-4,4-difluoro-3-methoxycyclohexyl]-4-methyl-6-oxohexahydropyrimidin-2-ylidene}carbamate

To a solution of Intermediate 24 (1.88 g, 2.88 mmol) in MeOH (30 mL) was added 20% Pd/C (0.23 g, 0.43 mmol) under an argon atmosphere. The reaction mixture was stirred at r.t. for 45 minutes under hydrogen pressure, then filtered through a pad of Celite®, washing through with MeOH (4×40 mL). The filtrate was dried over anhydrous Na₂SO₄ and concentrated in vacuo. The crude residue was purified by combi-flash chromatography (20% EtOAc in hexanes) to afford the title compound (1.21 g, 79%) as an off-white solid. δ_{H} (400 MHz, DMSO-d₆) 10.46 (s, 1H), 6.95-7.08 (m, 1H), 6.75-6.83 (m, 1H), 6.40-6.50 (m, 1H), 5.52 (s, 2H), 4.49-4.67 (m, 1H), 3.44-3.60 (m, 2H), 3.28-3.36 (s, 3H), 3.05-3.16 (m, 1H), 2.18-2.42 (m, 2H), 1.84-2.07 (m, 1H), 1.74-1.84 (m, 1H), 1.73 (s, 3H), 1.45 (s, 9H), 1.21-1.31 (m, 1H), 0.81-0.91 (m, 1H). LCMS (Method 1, ESI): 500.95 [MH]⁺, RT 2.06 minutes.

### Intermediate 26

### tert-Butyl (NE)-N-{(4S)-4-(2-chloro-3-iodophenyl)-1-[(1RS,3RS)-4,4-difluoro-3-methoxycyclohexyl]-4-methyl-6-oxohexahydropyrimidin-2-ylidene}carbamate

Prepared from Intermediate 25 (411 mg, 1.41 mmol) in accordance with the procedure described for Intermediate 1 to afford the title compound (55% UV purity) (777 mg, 65%) as a white solid. LCMS (Method 4, ESI): 612.2 [MH]⁺, RT 2.38 minutes.

### Intermediate 27

### [(4-Bromo-3-fluorophenyl)imino](dimethyl)(oxo)-λ⁶-sulfane

A suspension of 1-bromo-2-fluoro-4-iodobenzene (1.05 g, 3.38 mmol), (dimethane sulfinylidene)amine (320 mg, 3.43 mmol), Cs₂CO₃ (3.15 g, 9.66 mmol), Xantphos (0.2 g, 0.34 mmol) and Pd₂(dba)₃ (0.15 g, 0.16 mmol) in 1,4-dioxane (4 mL), purged under nitrogen, was stirred at 100° C. The reaction mixture was quenched with water (10 mL) and DCM (20 mL). The phases were separated, and the aqueous phase was washed with DCM (4×5 mL). The combined organic fractions were concentrated in vacuo. The crude residue was purified by flash chromatography (silica, 0-100% MeOH in DCM) to afford the title compound (0.88 g, 98%) as a dark brown solid. δ_{H} (300 MHz, CDCl₃) 7.32 (t, J 8.3 Hz, 1H), 6.85 (dd, J 10.5, 2.4 Hz, 1H), 6.74 (ddd, J 8.6, 2.5, 0.9 Hz, 1H), 3.13 (s, 6H). LCMS (Method 2, ESI): 267.8 [MH]⁺, RT 1.50 minutes.

### Intermediate 28

### {[3-Fluoro-4-(4,4,5,5-tetramethyl-1,3,2-dioxaborolan-2-yl)phenyl]imino}(dimethyl)-(oxo)-λ⁶-sulfane

Prepared from Intermediate 27 (0.88 g, 3.31 mmol) in accordance with the procedure described for Intermediate 3 to afford the title compound (80 mass %) (1.1 g, 85%) as a dark solid. δ_{H} (300 MHz, CDCl₃) 7.58 (dd, J 8.0, 7.2 Hz, 1H), 6.83 (dd, J 8.0, 1.9 Hz, 1H), 6.74 (dd, J 11.3, 1.9 Hz, 1H), 3.16 (s, 6H), 1.33 (s, 12H). LCMS (Method 3, ESI): 314.2 [MH]⁻, RT 1.44 minutes.

### Intermediate 29

### 1-Bromo-2-fluoro-4-(methylsulfonyl)benzene

To a solution of DL-proline (0.11 g, 0.97 mmol) in DMSO (4 mL) was added NaH (60 mass % in mineral oil) (39 mg, 0.975 mmol) and the mixture was stirred for 5 minutes at r.t. CuI (185 mg, 0.97 mmol), sodium methanesulfinate (1.32 g, 12.9 mmol) and 1-bromo-2-fluoro-4-iodobenzene (1 g, 3.22 mmol) were added. The resulting suspension was stirred at 100° C. for 3 h, then quenched with water (10 mL) and DCM (20 mL). The phases were separated, and the aqueous phase was washed with DCM (4×5 mL). The combined organic fractions were concentrated in vacuo. The crude residue was purified by flash chromatography (silica, 0-50% EtOAc in hexane) to afford the title compound (75 mass %) (0.49 g, 45%) as a pale yellow crystalline solid. LCMS (Method 2, ESI): 271.6 [M+H₂O]⁺, RT 1.43 minutes.

### Intermediate 30

### 2-[2-Fluoro-4-(methylsulfonyl)phenyl]-4,4,5,5-tetramethyl-1,3,2-dioxaborolane

Prepared from Intermediate 29 (75 mass %) (0.49 g, 1.66 mol) in accordance with the procedure described for Intermediate 3 to afford the title compound (50 mass %) (0.92 g, 90%) as a viscous dark oil. δ_{H} (300 MHz, CDCl₃) 7.82 (dd, J 7.8, 5.5 Hz, 1H), 7.58 (dd, J 7.8, 1.6 Hz, 1H), 7.47 (dd, J 8.2, 1.6 Hz, 1H), 2.94 (s, 3H), 1.24 (s, 12H). LCMS (Method 3, ESI): 318.2 [M+H₂O]⁺, RT 0.39 minutes.

### Intermediate 31

### tert-Butyl (NE)-N-{(4S)-4-(2-chloro-3-iodophenyl)-1-[(1S,3S)-4,4-difluoro-3-methyl-cyclohexyl]-4-methyl-6-oxohexahydropyrimidin-2-ylidene}carbamate

Prepared from tert-butyl (NE)-N-{(4S)-4-(3-amino-2-chlorophenyl)-1-[(1S,3S)-4,4-difluoro-3-methylcyclohexyl]-4-methyl-6-oxohexahydropyrimidin-2-ylidene}-carbamate (WO 2022/008639, Intermediate 42) (4.0 g, 8.2 mol) in accordance with the procedure described for Intermediate 1 to afford the title compound (2.1 g, 41%) as an off-white solid. LCMS (Method 5, ESI): 596.2 [MH]⁺, RT 1.92 minutes.

### Intermediate 32

### 1-Bromo-4-(dimethylphosphoryl)-2-fluorobenzene

A suspension of 1-bromo-2-fluoro-4-iodobenzene (1.05 g, 3.38 mmol), dimethyl-phosphine oxide (320 mg, 3.43 mmol), Cs₂CO₃ (3150 mg, 9.66 mmol), Xantphos (0.2 g, 0.34 mmol) and Pd₂(dba)₃ (0.15 g, 0.16 mmol) in 1,4-dioxane (4 mL), purged under nitrogen, was stirred at 100° C. The reaction mixture was quenched with water (10 mL) and DCM (20 mL). The phases were separated, and the aqueous phase was washed with DCM (4×5 mL). The combined organic fractions were concentrated in vacuo. The crude residue was purified by flash chromatography (silica, 0-100% MeOH in DCM) to afford the title compound (0.87 g, 90%) as a yellow crystalline solid. δ_{H} (300 MHz, CDCl₃) 7.58 (ddd, J 8.1, 6.5, 3.0 Hz, 1H), 7.43 (ddd, J 12.0, 8.3, 1.7 Hz, 1H), 7.29 (ddd, J 11.0, 8.0, 1.7 Hz, 1H), 1.70 (s, 3H), 1.66 (s, 3H). LCMS (Method 2, ESI): 253.8 [MH]⁺, RT 1.08 minutes.

### Intermediate 33

### 2-[4-(Dimethylphosphoryl)-2-fluorophenyl]-4,4,5,5-tetramethyl-1,3,2-dioxaborolane

Prepared from Intermediate 32 (0.87 g, 3.31 mmol) in accordance with the procedure described for Intermediate 3 to afford the title compound (50 mass %) (0.96 g, 46%) as a dark viscous oil. 6H (300 MHz, CDCl₃) 7.71 (ddd, J 7.5, 5.5, 3.8 Hz, 1H), 7.38-7.18 (m, 2H), 1.62 (s, 3H), 1.58 (s, 3H), 1.19 (s, 12H). LCMS (Method 3, ESI): 299.2 [MH]⁺, RT 0.40 minutes.

### Intermediate 34

### rac-(1R,3R)-3-[tert-Butyl(dimethyl)silyl]oxy-4,4-difluorocyclohexanamine

To a solution of Intermediate 16 (1.9 g, 4.95 mmol) in MeOH (20 mL) was added 10% Pd/C (0.26 g, 0.25 mol) at r.t. The reaction mixture was stirred at r.t. for 3 h under H₂, then filtered through a pad of Celite, washing through with MeOH (3×30 mL). The filtrate was concentrated in vacuo to afford the crude title compound (1.20 g) as a colourless oil, which was utilised without further purification. δ_{H} (400 MHz, DMSO-d₆) 3.78-3.95 (m, 1H), 2.70-2.80 (m, 1H), 1.51-2.04 (m, 6H), 1.09-1.38 (m, 2H), 0.86 (s, 9H), 0.05 (s, 6H).

### Intermediate 35

### tert-Butyl N-({rac-(1R,3R)-3-[tert-Butyl(dimethyl)silyl]oxy-4,4-difluorocyclohexyl}-carbamothioyl)carbamate

Prepared from Intermediate 34 (1.00 g, 3.77 mol) in accordance with the procedure described in WO 2022/008639 (for Intermediate 2 thereof) to afford the title compound (1.51 g, 71%) as a pale-brown liquid. LCMS (Method 1, ESI): 368.95 [(M-tBu)+H]⁺, RT 2.63 minutes.

### Intermediates 36 & 37

### tert-Butyl (NE)-N-[(4S)-4-[3-(benzyloxycarbonylamino)-2-chlorophenyl]-1-{(1S*,3S*)-3-[tert-butyl(dimethyl)silyl]oxy-4,4-difluorocyclohexyl}-4-methyl-6-oxohexahydro-pyrimidin-2-ylidene]carbamate (Intermediate 36)

### tert-Butyl (NE)-N-[(4S)-4-[3-(benzyloxycarbonylamino)-2-chlorophenyl]-1-{(1R*,3R*)-3-[tert-butyl(dimethyl)silyl]oxy-4,4-difluorocyclohexyl}-4-methyl-6-oxohexahydro-pyrimidin-2-ylidene]carbamate (Intermediate 37)

Prepared from Intermediate 35 (1.25 g, 2.20 mmol) and methyl (3S)-3-amino-3-[3-(benzyloxycarbonylamino)-2-chlorophenyl]butanoate (WO 2022/008639, Intermediate 7) (0.91 g, 2.20 mol) in accordance with the procedure described in WO 2022/008639 (for Intermediate 8 thereof), and purified by flash chromatography, to afford the title compounds (Intermediate 36, 0.52 g, 39%; and Intermediate 37, 0.53 g, 40%) as off-white solids.

Intermediate 36: δ_{H} (400 MHz, DMSO-d₆) 10.47 (s, 1H), 9.24 (s, 1H), 7.59 (d, J 7.83 Hz, 1H), 7.37-7.43 (m, 4H), 7.27-7.36 (m, 2H), 7.16 (d, J 8.31 Hz, 1H), 5.14 (s, 2H), 4.56 (t, J 12.23 Hz, 1H), 3.78-3.93 (m, 1H), 3.58 (d, J 16.63 Hz, 1H), 3.12-3.26 (m, 1H), 2.38-2.48 (m, 1H), 2.19-2.36 (m, 1H), 1.82-1.94 (m, 1H), 1.75 (s, 3H), 1.54-1.68 (m, 2H), 1.44 (s, 9H), 0.98-1.08 (m, 1H), 0.84 (s, 9H), 0.04 (s, 6H). LCMS (Method 1, ESI): 735.35 [MH]⁺, RT 2.74 minutes.

Intermediate 37: δ_{H} (400 MHz, DMSO-d₆) 10.50 (s, 1H), 9.23 (s, 1H), 7.58 (d, J 8.31 Hz, 1H), 7.37-7.43 (m, 4H), 7.26-7.36 (m, 2H), 7.16 (d, J 7.83 Hz, 1H), 5.14 (s, 2H), 4.38-4.51 (m, 1H), 3.71-3.88 (m, 1H), 3.50-3.63 (m, 1H), 3.14-3.24 (m, 1H), 2.24-2.41 (m, 2H), 1.87-1.97 (m, 1H), 1.73-1.78 (m, 3H), 1.54-1.71 (m, 2H), 1.44 (s, 9H), 1.00-1.12 (m, 1H), 0.79 (s, 9H), −0.03 (s, 6H). LCMS (Method 1, ESI): 735.25 [MH]⁺, RT 2.70 minutes.

### Intermediate 38

### tert-Butyl (NE)-N-[(4S)-4-(3-amino-2-chlorophenyl)-1-{(1S*,3S*)-3-[tert-butyl-(dimethyl)silyl]oxy-4,4-difluorocyclohexyl}-4-methyl-6-oxohexahydropyrimidin-2-ylidene]carbamate

Prepared from Intermediate 36 (0.50 g, 0.65 mmol) in accordance with the procedure described in WO 2022/008639 (for Intermediate 9 thereof) to afford the title compound (0.32 g, 77%) as an off-white solid. δ_{H} (400 MHz, DMSO-d₆) 10.42 (s, 1H), 6.92-7.04 (m, 1H), 6.78 (d, J 7.82 Hz, 1H), 6.46 (d, J 7.83 Hz, 1H), 5.53 (br s, 2H), 4.58 (t, J 11.74 Hz, 1H), 3.78-3.94 (m, 1H), 3.44-3.57 (m, 1H), 3.06-3.17 (m, 1H), 2.37-2.48 (m, 1H), 2.21-2.37 (m, 1H), 1.82-1.96 (m, 1H), 1.76-1.81 (m, 1H), 1.72 (s, 3H), 1.56-1.66 (m, 1H), 1.44 (s, 9H), 1.04-1.13 (m, 1H), 0.84 (s, 9H), 0.04 (s, 6H). LCMS (Method 1, ESI): 601.15 [MH]⁺, RT 2.58 minutes.

### Intermediate 39

### tert-Butyl (NE)-N-[(4S)-1-{(1S*,3S*)-3-[tert-butyl(dimethyl)silyl]oxy-4,4-difluoro-cyclohexyl}-4-(2-chloro-3-iodophenyl)-4-methyl-6-oxohexahydropyrimidin-2-ylidene]-carbamate

Prepared from Intermediate 38 (0.35 g, 0.56 mmol) in accordance with the procedure described for Intermediate 1 to afford the title compound (0.23 g, 48%) as an off-white solid. LCMS (Method 1, ESI): 711.85 [MH]⁺, RT 1.96 minutes.

### Intermediate 40

### tert-Butyl (NE)-N-[(4S)-4-(3-amino-2-chlorophenyl)-1-{(1R*,3R*)-3-[tert-butyl-(dimethyl)silyl]oxy-4,4-difluorocyclohexyl}-4-methyl-6-oxohexahydropyrimidin-2-ylidene]carbamate

Prepared from Intermediate 37 (0.50 g, 0.65 mmol) in accordance with the procedure described in WO 2022/008639 (for Intermediate 9 thereof) to afford the title compound (0.33 g, 80%) as an off-white solid. δ_{H} (400 MHz, DMSO-d₆) 10.46 (s, 1H), 6.88-7.00 (m, 1H), 6.77 (d, J 7.82 Hz, 1H), 6.46 (d, J 7.34 Hz, 1H), 5.52 (s, 2H), 4.39-4.54 (m, 1H), 3.84 (dd, J 15.41, 4.16 Hz, 1H), 3.50 (d, J 16.63 Hz, 1H), 3.06-3.18 (m, 1H), 2.26-2.40 (m, 2H), 1.88-1.97 (m, 1H), 1.75-1.84 (m, 1H), 1.73 (s, 3H), 1.48-1.53 (m, 1H), 1.45 (s, 9H), 1.05-1.16 (m, 1H), 0.81 (s, 9H), −0.01 (s, 6H). LCMS (Method 1, ESI): 601.10 [MH]⁺, RT 2.54 minutes.

### Intermediate 41

### tert-Butyl (NE)-N-[(4S)-1-{(1R*,3R*)-3-[tert-butyl(dimethyl)silyl]oxy-4,4-difluoro-cyclohexyl}-4-(2-chloro-3-iodophenyl)-4-methyl-6-oxohexahydropyrimidin-2-ylidene]-carbamate

Prepared from Intermediate 40 (1.7 g, 2.77 mmol) in accordance with the procedure described for Intermediate 1 to afford the title compound (1.10 g, 49%) as an off-white solid. δ_{H} (400 MHz, DMSO-d₆) 10.49 (s, 1H), 7.96 (d, J 7.82 Hz, 1H), 7.29-7.43 (m, 1H), 7.00-7.12 (m, 1H), 4.34-4.50 (m, 1H), 3.75-3.94 (m, 1H), 3.58 (d, J 17.12 Hz, 1H), 3.15-3.23 (m, 1H), 2.20-2.41 (m, 2H), 1.86-1.97 (m, 1H), 1.75-1.83 (m, 1H), 1.73 (s, 3H), 1.59-1.67 (m, 1H), 1.45 (s, 9H), 0.93-1.01 (m, 1H), 0.80 (s, 9H), −0.02 (s, 6H). LCMS (Method 1, ESI): 712.05 [MH]⁺, RT 2.71 minutes.

### Intermediate 42

### tert-Butyl (NE)-N-[(4S)-1-{(1R*,3R*)-3-[tert-butyl(dimethyl)silyl]oxy-4,4-difluoro-cyclohexyl}-4-[2-chloro-3-(3-chlorophenyl)phenyl]-4-methyl-6-oxohexahydropyrimidin-2-ylidene]carbamate

To a solution of Intermediate 41 (0.62 g, 0.87 mmol) in 1,4-dioxane (20 mL) and water (5 mL) were added (3-chlorophenyl)boronic acid (0.14 g, 0.87 mmol), K₃PO₄ (0.55 g, 2.61 mmol) and Pd(dppf)Cl₂ (0.06 g, 0.09 mmol). The reaction mixture was purged with argon for 10 minutes and heated at 80° C. for 2 h, then concentrated in vacuo. The residue was diluted with water (50 mL) and extracted with EtOAc (3×50 mL). The organic layer was separated, dried over anhydrous sodium sulfate and concentrated in vacuo. The crude residue was purified by flash chromatography (silica, 0-25% EtOAc in hexane) to afford the title compound (0.59 g, 86%) as an off-white solid. LCMS (Method 1, ESI): 696.30 [MH]⁺, RT 2.91 minutes.

### Intermediate 43

### tert-Butyl (NE)-N-{(4S)-4-[2-chloro-3-(3-chlorophenyl)phenyl]-1-[(1R*,3R*)-4,4-difluoro-3-hydroxycyclohexyl]-4-methyl-6-oxohexahydropyrimidin-2-ylidene}carbamate

To a solution of Intermediate 42 (0.58 g, 0.83 mol) in THF (15 mL) was added 1M TBAF (0.83 mL, 0.83 mmol) at 0° C. over 10 minutes. The reaction mixture was stirred at r.t. for 1.5 h, then concentrated in vacuo. The residue was diluted with water (30 mL) and extracted with EtOAc (3×30 mL). The organic layer was separated, dried over anhydrous sodium sulfate and concentrated in vacuo. The crude residue was purified by flash chromatography (silica, 0-40% EtOAc in hexane) to afford the title compound (0.42 g, 86%) as an off-white solid. LCMS (Method 1, ESI): 582.15 [MH]⁺, RT 2.51 minutes.

### Intermediate 44

### tert-Butyl (NE)-N-{(4S)-1-[(1R*,3S*)-3-benzyloxy-4,4-difluorocyclohexyl]-4-[2-chloro-3-(3-chlorophenyl)phenyl]-4-methyl-6-oxohexahydropyrimidin-2-ylidene}carbamate

To a solution of Intermediate 43 (110 mg, 0.18 mmol) in toluene (10 mL) was added (cyanomethylene)tributylphosphorane (130 mg, 0.54 mmol), followed by phenyl-methanol (20.0 mg, 0.22 mmol). The reaction mixture was heated at 60° C. for 12 h, then concentrated in vacuo. The crude residue was purified by preparative HPLC to afford the title compound (35.0 mg, 29%) as an off-white solid. LCMS (Method 1, ESI): 671.88 [MH]⁺, RT 1.88 minutes.

### Intermediate 45

### tert-Butyl (NE)-N-{(4S)-4-[2-chloro-3-(3-chlorophenyl)phenyl]-1-[(1R*,3S*)-4,4-difluoro-3-hydroxycyclohexyl]-4-methyl-6-oxohexahydropyrimidin-2-ylidene}carbamate

To a solution of Intermediate 44 (30.0 mg, 0.05 mmol) in MeOH (5 mL) was added 10% Pd/C (80 mg) at 0° C. The reaction mixture was stirred at r.t. for 2 h under a H₂ atmosphere, then filtered through a pad of Celite, washing through with MeOH (2×20 mL). The filtrate was concentrated in vacuo to afford the crude title compound (24.0 mg) as a colourless liquid. LCMS (Method 1, ESI): 584.10 [MH]⁺, RT 2.31 minutes.

### Intermediate 46

### tert-Butyl (NE)-N-{(4S)-4-[2-chloro-3-(3-formylphenyl)phenyl]-4-methyl-1-[(2S,4S)-2-methyltetrahydropyran-4-yl]-6-oxohexahydropyrimidin-2-ylidene}carbamate

To a suspension of Intermediate 1 (1.0 g, 1.53 mmol) in 1,4-dioxane (5 mL) and water (0.5 mL) were added 3-formylphenylboronic acid (685 mg, 4.57 mmol) and Cs₂CO₃ (2 g, 6.13 mmol) at r.t. The reaction mixture was purged with argon for 10 minutes, then Pd(dppf)Cl₂ (225 mg, 0.29 mmol) was added at r.t. The reaction mixture was purged with nitrogen for 5 minutes and heated in a sealed tube at 60° C. for 1.5 h, then quenched with water (10 mL) and DCM (25 mL). The phases were separated, and the aqueous phase was washed with DCM (4×5 mL). The combined organic fractions were concentrated in vacuo. The crude residue was purified by flash chromatography (silica, 0-100% EtOAc in hexane) to afford the title compound (0.68 g, 83%) as a pale yellow solid. δ_{H} (300 MHz, DMSO-d₆) 10.53 (d, J 1.5 Hz, 1H), 10.08 (s, 1H), 7.97 (td, J 4.4, 1.6 Hz, 1H), 7.89 (q, J 1.4 Hz, 1H), 7.77-7.67 (m, 2H), 7.57-7.35 (m, 3H), 4.80-4.60 (m, 1H), 3.79 (dd, J 11.6, 4.4 Hz, 1H), 3.70-3.54 (m, 1H), 3.28-3.19 (m, 2H), 2.37-2.22 (m, 1H), 2.11 (q, J 11.9 Hz, 1H), 1.84 (s, 3H), 1.45 (s, 9H), 1.36-0.96 (m, 5H), 0.91-0.75 (m, 1H). LCMS (Method 3, ESI): 540.2 [MH]⁺, RT 2.70 minutes.

### Intermediate 47

### tert-Butyl (NE)-N-[(4S)-4-(2-chloro-3-{3-[(1SR)-2,2-difluoro-1-hydroxyethyl]phenyl}-phenyl)-4-methyl-1-[(2S,4S)-2-methyltetrahydropyran-4-yl]-6-oxohexahydropyrimidin-2-ylidene]carbamate

To a suspension of Intermediate 46 (0.23 g, 0.43 mmol) in THF (3 mL) and DMF (1 mL) were added caesium fluoride (200 mg, 1.32 mmol) and (difluoromethyl)trimethyl-silane (0.2 mL, 1 mmol) at r.t. The reaction mixture was stirred at r.t. for 2 h, then 60° C. for 20 h, then quenched with saturated aqueous NaHCO₃ solution (10 mL) and diluted with EtOAc (10 mL). The phases were separated, and the aqueous phase was washed with EtOAc (2×10 mL). The combined organic fractions were concentrated in vacuo. The crude residue was treated with a solution of TBAF (IM in THF) (3 mL, 3 mmol) for 30 minutes, then the crude mixture was concentrated in vacuo. The residue was purified by flash chromatography (silica, 0-80% EtOAc in hexane) to afford the title compound (0.22 g, 88%) as a pale yellow solid. LCMS (Method 3, ESI): 592.2 [MH]⁺, RT 2.56 minutes.

### Intermediates 48 & 49

### tert-Butyl (NE)-N-[(4S)-4-{2-chloro-3-[3-(1R*)(2,2-difluoro-1-hydroxyethyl)phenyl]-phenyl}-4-methyl-1-[(2S,4S)-2-methyltetrahydropyran-4-yl]-6-oxohexahydropyrimidin-2-ylidene]carbamate (Intermediate 48)

### tert-Butyl (NE)-N-[(4S)-4-{2-chloro-3-[3-(1S*)(2,2-difluoro-1-hydroxyethyl)phenyl]-phenyl}-4-methyl-1-[(2S,4S)-2-methyltetrahydropyran-4-yl]-6-oxohexahydropyrimidin-2-ylidene]carbamate (Intermediate 49)

Intermediate 47 was subjected to chiral preparative HPLC (Column: Chiralpak IC 250×20 mm, 5 μm; Method: isocratic 10% MeOH (no additive) over 12 minutes; Temperature: 40° C.; Flow: 100 mL/minute; ABPR: 60 bar) to afford Intermediate 48 (29 mg, 25%) and Intermediate 49 (29 mg, 25%).

Intermediate 48: SFC chiral LCMS peak 1, RT 5.44 minutes (dr=99:1).

Intermediate 49: SFC chiral LCMS peak 2, RT 6.13 minutes (dr=10:90).

SFC chiral LCMS conditions:—Column: Chiralpak IC 150×4.6 mm, 3 μm; Temperature: 35° C.; Flow Rate: 3 mL/minute; Method: isocratic 10% MeOH (no additive) over 10 minutes.

### Intermediate 50

### tert-Butyl (NE)-N-[(4S)-4-(2-chloro-3-{3-[(1SR)-2,2,2-trifluoro-1-hydroxyethyl]phenyl}-phenyl)-4-methyl-1-[(2S,4S)-2-methyltetrahydropyran-4-yl]-6-oxohexahydropyrimidin-2-ylidene]carbamate

To a suspension of Intermediate 46 (0.23 g, 0.43 mmol) in THF (3 mL) and DMF (1 mL) were added caesium fluoride (200 mg, 1.32 mmol) and (trifluoromethyl)trimethyl-silane (98 mass %) (0.2 mL, 1 mmol) at r.t. The reaction mixture was stirred at r.t. for 2 h, then quenched with saturated aqueous NaHCO₃ solution (10 mL) and diluted with EtOAc (10 mL). The phases were separated, and the aqueous phase was washed with EtOAc (2×10 mL). The combined organic fractions were concentrated in vacuo. The crude residue was treated with a solution of TBAF (1M in THF) (3 mL, 3 mmol) for 30 minutes, then the crude mixture was concentrated in vacuo. The residue was purified by flash chromatography (silica, 0-80% EtOAc in hexane) to afford the title compound (0.27 g, 90%) as a pale yellow solid. LCMS (Method 3, ESI): 608.2 [M−H]⁺, RT 1.36 minutes.

### Intermediates 51 & 52

### tert-Butyl (NE)-N-[(4S)-4-{2-chloro-3-[3-(1R*)(2,2,2-trifluoro-1-hydroxyethyl)phenyl]-phenyl}-4-methyl-1-[(2S,4S)-2-methyltetrahydropyran-4-yl]-6-oxohexahydropyrimidin-2-ylidene]carbamate (Intermediate 51)

### tert-Butyl (NE)-N-[(4S)-4-{2-chloro-3-[3-(1S*)-(2,2,2-trifluoro-1-hydroxyethyl)phenyl]-phenyl}-4-methyl-1-[(2S,4S)-2-methyltetrahydropyran-4-yl]-6-oxohexahydropyrimidin-2-ylidene]carbamate (Intermediate 52)

Intermediate 50 was subjected to chiral preparative HPLC (Column: Chiralpak IC 250×20 mm, 5 μm; Method: isocratic 10% MeOH (no additive) over 12 minutes; Temperature: 40° C.; Flow: 100 mL/minute; ABPR: 60 bar) to afford Intermediate 51 (20 mg, 15%) and Intermediate 52 (20 mg, 15%).

Intermediate 51: SFC chiral LCMS peak 1, RT 3.16 minutes (dr=97:3).

Intermediate 52: SFC chiral LCMS peak 2, RT 3.48 minutes (dr=7:93).

SFC chiral LCMS conditions:—Column: Chiralpak IC 150×4.6 mm, 3 μm; Temperature: 35° C.; Flow Rate: 3 mL/minute; Method: isocratic 10% MeOH (no additive) over 6.5 minutes.

### Intermediate 53

### Methyl (3S)-3-[3-(benzyloxycarbonylamino)-2-chlorophenyl]-3-(tert-butoxycarbonyl-amino)butanoate

To a solution of methyl (3S)-3-amino-3-[3-(benzyloxycarbonylamino)-2-chloro-phenyl]butanoate (WO 2022/008639, Intermediate 7) (7.1 g, 17.2 mmol) in THF (20 mL) were added di-tert-butyl dicarbonate (4.3 g, 19.7 mmol), sodium bicarbonate (4.4 g, 52.1 mmol) and water (3 mL) at r.t. The reaction mixture was stirred at r.t. for 16 h, then quenched with water (50 mL) and extracted with EtOAc (3×50 mL). The organic layer was separated and dried over anhydrous sodium sulfate, then concentrated in vacuo. The crude residue was purified by flash chromatography (silica, 0-50% EtOAc in hexane) to afford the title compound (7.02 g, 82%) as a colourless liquid. δ_{H} (300 MHz, CDCl₃) 8.16 (dd, J 8.1, 1.6 Hz, 1H), 7.33-7.50 (m, 5H), 7.28 (dd, J 8.1, 8.1 Hz, 2H), 7.17 (dd, J 8.1, 1.6 Hz 1H), 5.81 (s, 1H), 5.24 (s, 2H), 3.68 (s, 3H), 3.42 (d, J 14.1 Hz, 1H), 2.80 (d, J 14.1 Hz, 1H), 1.91 (s, 3H), 1.36 (s, 9H). LCMS (Method 2, ESI): 475.2 [M−H]⁺, RT 2.40 minutes.

### Intermediate 54

### Methyl (3S)-3-(3-amino-2-chlorophenyl)-3-(tert-butoxycarbonylamino)butanoate

Prepared from Intermediate 53 (2.7 g, 5.6 mmol) in accordance with the procedure described in WO 2022/008639 (for Intermediate 9 thereof) to afford the title compound (2.0 g, 99%) as a colourless oil. δ_{H} (300 MHz, CDCl₃) 7.09 (dd, J 7.9, 7.9 Hz, 1H), 6.81-6.96 (m, 2H), 5.79 (s, 1H), 3.72 (s, 2H), 3.69 (s, 3H), 3.47 (d, J 14.1 Hz, 1H), 2.83 (d, J 14.1 Hz, 1H), 1.91 (s, 3H), 1.37 (s, 9H). LCMS (Method 3, ESI): 243.2 [MH-BOC]⁺, RT 1.03 minutes.

### Intermediate 55

### Methyl (3S)-3-(tert-butoxycarbonylamino)-3-(2-chloro-3-iodophenyl)butanoate

Prepared from Intermediate 54 (3.2 g, 8.9 mmol) in accordance with the procedure described for Intermediate 1 to afford the title compound (89 mass %) (1.6 g, 36%) as a colourless oil. LCMS (Method 3, ESI): 354.0 [MH-BOC]*, RT 2.43 minutes.

### Intermediate 56

### Methyl (3S)-3-amino-3-(2-chloro-3-iodophenyl)butanoate hydrochloride

To a solution of Intermediate 55 (89 mass %) (1.2 g, 2.4 mmol) in 1,4-dioxane (10 mL) was added 4N HCl in 1,4-dioxane (10 mL). The reaction mixture was stirred at r.t. for 46 h, then concentrated in vacuo, to afford the title compound (89 mass %) (1.04 g, 100%) as a solid. LCMS (Method 3, ESI): 354.0 [MH]⁺, RT 0.99 minutes.

### Intermediate 57

### tert-Butyl N-[(2-methyl-1,1-dioxothian-4-yl)carbamothioyl]carbamate

Prepared from 2-methyl-1,1-dioxothian-4-amine hydrochloride (1.0 g, 5 mol) in accordance with the procedure described in WO 2022/008639 (for Intermediate 2 thereof) to afford the title compound (1.2 g, 78%) as a white solid. LCMS (Method 3, ESI): 321.0 [MH]⁻, RT 1.50 minutes.

### Intermediate 58

### tert-Butyl (NE)-N-[(4S)-4-(2-chloro-3-iodophenyl)-4-methyl-1-(2-methyl-1,1-dioxothian-4-yl)-6-oxohexahydropyrimidin-2-ylidene]carbamate

Prepared from Intermediate 57 (1.2 g, 3.7 mmol) and Intermediate 56 (1.45 g, 3.7 mmol) in accordance with the procedure described in WO 2022/008639 (for Intermediate 8 thereof) to afford the title compound (70 mass %) (1.6 g, 49%) as a white solid. LCMS (Method 3, ESI): 610.0 [MH]⁺, RT 2.50 minutes.

### Intermediate 59

### tert-Butyl (NE)-N-{(4S)-4-(2-chloro-3-iodophenyl)-1-[(1RS,3RS,4SR)-4-hydroxy-3-methylcyclohexyl]-4-methyl-6-oxohexahydropyrimidin-2-ylidene}carbamate

Prepared from Intermediate 7 (880 mg, 3.05 mmol) and Intermediate 56 (1.04 g, 2.67 mmol) in accordance with the procedure described in WO 2022/008639 (for Intermediate 8 thereof) to afford the title compound (60% LUV purity) (1.14 g, 50%) as a pale yellow solid. LCMS (Method 3, ESI): 576.0 [MH]⁺, RT 1.36 minutes.

### Intermediate 60

### tert-Butyl (NE)-N-[(4S)-1-{(1SR,3SR)-3-[tert-butyl(dimethyl)silyl]oxy-4,4-difluoro-cyclohexyl}-4-(2-chloro-3-iodophenyl)-4-methyl-6-oxohexahydropyrimidin-2-ylidene]-carbamate

Prepared from Intermediate 35 (312 mg, 0.74 mmol) and Intermediate 56 (450 mg, 1.15 mmol) in accordance with the procedure described in WO 2022/008639 (for Intermediate 8 thereof) to afford the title compound (166 mg, 30%) as a white solid. LCMS (Method 3, ESI): 712.2 [MH]⁺, RT 1.77 minutes.

### Intermediate 61

### tert-Butyl N-{[3-(dimethylamino)cyclobutyl]carbamothioyl}carbamate

Prepared from N¹,N¹-dimethylcyclobutane-1,3-diamine dihydrochloride (500.0 mg, 4.7 mmol) in accordance with the procedure described in WO 2022/008639 (for Intermediate 2 thereof) to afford the title compound (240 mg, 33%) as a white solid. LCMS (Method 2, ESI): 274.0 [MH]⁺, RT 1.12 minutes.

### Intermediate 62

### tert-Butyl (NE)-N-{(4S)-4-(2-chloro-3-iodophenyl)-1-[3-(dimethylamino)cyclobutyl]-4-methyl-6-oxohexahydropyrimidin-2-ylidene}carbamate

Prepared from Intermediate 61 (200.0 mg, 0.34 mmol) and Intermediate 56 in accordance with the procedure described in WO 2022/008639 (for Intermediate 8 thereof) to afford the title compound (410.3 mg), which was utilised without further purification. LCMS (Method 3, ESI): 561.0 [MH]⁺, RT 1.31 minutes.

### Intermediate 63

### tert-Butyl N-[(1-methylpiperidin-4-yl)carbamothioyl]carbamate

Prepared from 4-amino-1-methylpiperidine (0.16 g, 1.24 mmol) in accordance with the procedure described in WO 2022/008639 (for Intermediate 2 thereof) to afford the title compound (0.36 g, 95%) as a pale grey solid. LCMS (Method 3, ESI): 274.2 [MH]⁺, RT 1.47 minutes.

### Intermediate 64

### tert-Butyl (NE)-N-[(4S)-4-(2-chloro-3-iodophenyl)-4-methyl-1-(1-methylpiperidin-4-yl)-6-oxohexahydropyrimidin-2-ylidene]carbamate

Prepared from Intermediate 63 (0.14 g, 3.7 mmol) and Intermediate 56 in accordance with the procedure described in WO 2022/008639 (for Intermediate 8 thereof) to afford the title compound (80 mass %) (0.21 g, 73%) as a white solid. LCMS (Method 3, ESI): 561.2 [MH]⁺, RT 2.61 minutes.

### Intermediate 65

### Methyl (3S)-3-(tert-butoxycarbonylamino)-3-[2-chloro-3-(3-chlorophenyl)phenyl]-butanoate

Intermediate 55 (1.1 g, 2.3 mol), 3-chlorophenylboronic acid (1.08 g, 6.9 mmol), Pd(dppf)Cl₂ (177.0 mg, 2.3 mol) and cesium carbonate (2.25 g, 6.9 mol) were dissolved in 1,4-dioxane (10 mL) and water (1.0 mL). The reaction mixture was stirred at 60° C. for 1.5 h under a N₂ atmosphere, then filtered through a pad of Celite. The filtrate was concentrated in vacuo. The crude residue was purified by flash chromatography (silica, 0-30% EtOAc in hexane) to afford the title compound (990 mg, 96%) as a pale red oil. LCMS (Method 3, ESI): 338.2/340.2 [MH-BOC]⁺, RT 1.43 minutes.

### Intermediate 66

### Methyl (3S)-3-amino-3-[2-chloro-3-(3-chlorophenyl)phenyl]butanoate hydrochloride

To a solution of Intermediate 65 (985 mg, 2.25 mol) in 1,4-dioxane (10 mL) was added 4M hydrochloric acid in 1,4-dioxane (10 mL). The reaction mixture was stirred at r.t. for 18 h, then concentrated in vacuo, to afford the title compound (HCl salt) (88% LV purity) (867 mg, 91%) as a pale yellow powder. LCMS (Method 3, ESI): 338.2/340.2 [MH]⁺, RT 1.19 minutes.

### Intermediate 67

### Methyl (3S)-3-(tert-butoxycarbonylamino)-3-[2-chloro-3-(2,4-difluorophenyl)phenyl]-butanoate

Prepared from Intermediate 55 (1.00 g, 2.08 mmol) and (2,4-difluorophenyl)-boronic acid (0.99 g, 6.25 mol) in accordance with the procedure described for Intermediate 65 to afford the title compound (0.75 g, 72%) as a pale yellow semi-solid. δ_{H} (400 MHz, DMSO-d₆) 7.47 (d, J 7.83 Hz, 1H), 7.13-7.40 (m, 6H), 3.50 (s, 3H), 3.12-3.28 (m, 2H), 1.69-1.82 (m, 3H), 1.32 (s, 9H). LCMS (Method 1, ESI): 339.90 [MH-BOC]⁺, RT 2.02 minutes.

### Intermediate 68

### Methyl 3(S)-3-amino-3-[2-chloro-3-(2,4-difluorophenyl)phenyl]butanoate hydrochloride

Prepared from Intermediate 67 (0.7 g, 1.49 mmol) in accordance with the procedure described for Intermediate 66 to afford the title compound (0.50 g, 82%) as a pale yellow solid. δ_{H} (400 MHz, DMSO-d₆) 9.08 (br s, 3H), 7.56-7.63 (m, 1H), 7.47-7.55 (m, 1H), 7.30-7.45 (m, 3H), 7.14-7.25 (m, 1H), 3.58-3.73 (m, 1H), 3.49 (s, 3H), 3.35-3.42 (m, 1H), 1.80-1.94 (m, 3H). LCMS (Method 1, ESI): 340.17 [MH]⁺, RT 2.08 minutes.

### Intermediate 69

### Methyl (3S)-3-(tert-butoxycarbonylamino)-3-[2-chloro-3-(6-cyclopropylpyridin-3-yl)-phenyl]butanoate

Prepared from Intermediate 55 (700.0 mg, 1.54 mmol) and 6-cyclopropylpyridin-3-ylboronic acid (327 mg, 6.25 mmol) in accordance with the procedure described for Intermediate 65 to afford the title compound (430 mg, 63%) as a pale yellow gum.

LCMS (Method 5, ESI): 445.2 [MH]⁺, RT 1.64 minutes.

### Intermediate 70

### Methyl 3(S)-3-amino-3-[2-chloro-3-(6-cyclopropylpyridin-3-yl)phenyl]butanoate hydrochloride

Prepared from Intermediate 69 (430 mg, 0.97 mol) in accordance with the procedure described for Intermediate 66 to afford the title compound (420 mg, 100%) as a yellow solid. LCMS (Method 5, ESI): 345.1 [MH]⁺, RT 1.34 minutes.

### Intermediate 71

### tert-Butyl N-[(1-cyclopropyl-2-hydroxyethyl)carbamothioyl]carbamate

Prepared from 2-amino-2-cyclopropylethanol hydrochloride (112 mg, 2.50 mmol) in accordance with the procedure described in WO 2022/008639 (for Intermediate 2 thereof) to afford the title compound (105 mg, 47%) as a pale yellow oil.

### Intermediate 72

### tert-Butyl N-({2-[tert-butyl(dimethyl)silyl]oxy-1-cyclopropylethyl}carbamothioyl)-carbamate

To a solution of Intermediate 71 (100 mg, 0.384 mmol) in DMF (1 mL) were added imidazole (136 mg, 1.98 mmol) and tert-butyldimethylchlorosilane (105 mg, 0.676 mmol). The reaction mixture was stirred at r.t. for 72 h, then purified by flash chromatography (silica, 0-30% EtOAc in hexane), to afford the title compound (93 mg, 63%) as a colourless oil. δ_{H} (400 MHz, CDCl₃) 10.09 (s, 1H), 7.77 (s, 1H), 3.77-3.96 (m, 2H), 1.51 (s, 9H), 1.19-1.31 (m, 1H), 0.93 (s, 9H), 0.48-0.71 (m, 4H), 0.29-0.44 (m, 1H), 0.09 (s, 3H), 0.08 (s, 3H). LCMS (Method 2, ESI): 375.0 [MH]⁺, RT 1.82 minutes.

### Intermediate 73

### tert-Butyl N-{[(3RS,4SR)-3-hydroxytetrahydropyran-4-yl]carbamothioyl}carbamate

Prepared from trans-4-aminotetrahydropyran-3-ol (300 mg, 2.56 mmol) in accordance with the procedure described in WO 2022/008639 (for Intermediate 2 thereof) to afford the title compound (500 mg, 67%) as a white solid. LCMS (Method 3, ESI): 277.2 [MH]⁺, RT 0.76 minutes.

### Intermediate 74

### tert-Butyl N-({(3RS,4SR)-3-[tert-butyl(dimethyl)silyl]oxytetrahydropyran-4-yl}-carbamothioyl)carbamate

Prepared from Intermediate 73 (411 mg, 1.41 mmol) in accordance with the procedure described for Intermediate 72 to afford the title compound (534 mg, 95%) as a colourless oil. LCMS (Method 3, ESI): 391.2 [MH]⁺, RT 1.47 minutes.

### Intermediate 75

### tert-Butyl rac-(2S,4S)-4-(tert-butoxycarbonylcarbamothioylamino)-2-methylpiperidine-1-carboxylate

Prepared from tert-butyl rac-(2S,4S)-4-amino-2-methylpiperidine-1-carboxylate (1.5 g, 7.0 mmol) in accordance with the procedure described in WO 2022/008639 (for Intermediate 2 thereof) to afford the title compound (2.60 g, 78%) as an off-white solid.

LCMS (Method 1, ESI): 372.30 [M−H]⁺, RT 2.31 minutes.

### Intermediate 76

### rac-(1S,2S,4S)-4-Amino-2-methylcyclohexanol trifluoroacetic acid salt

A solution of Intermediate 5 (801.0 mg, 3.49 mmol) was stirred in TFA (2 mL) and DCM (8 mL) at r.t. for 1 h, then concentrated in vacuo, to obtain the title compound (800 mg, 94%) as a white solid, which was utilised without further purification.

### Intermediate 77

### tert-Butyl N-{[rac-(1S,3S,4S)-4-hydroxy-3-methylcyclohexyl]carbamothioyl}carbamate

Prepared from Intermediate 76 (800 mg, 3.49 mmol) in accordance with the procedure described in WO 2022/008639 (for Intermediate 2 thereof) to afford the title compound (810 mg, 52%) as a pale yellow gel. LCMS (Method 3, ESI): 289.2 [MH]⁺, RT 1.62 minutes.

### Intermediate 78

### tert-Butyl N-({rac-(1S,3S,4S)-4-[tert-butyl(dimethyl)silyl]oxy-3-methylcyclohexyl}-carbamothioyl)carbamate

Prepared from Intermediate 77 (810 mg, 2.81 mmol) in accordance with the procedure described for Intermediate 72 to afford the title compound (1.12 g, 99%) as a white solid. δ_{H} (300 MHz, CDCl₃) 9.54 (d, J 8.0 Hz, 1H), 8.46 (s, 1H), 4.20 (dp, J 11.6, 3.9 Hz, 1H), 3.16-3.02 (m, 1H), 2.72 (s, 1H), 2.20-2.02 (m, 2H), 1.85 (dq, J 12.6, 3.5 Hz, 1H), 0.86 (d, J 7.2 Hz, 15H), 0.03-0.00 (m, 9H). LCMS (Method 3, ESI): 403.2 [MH]⁺, RT 3.67 minutes.

### Intermediate 79

### (6S)-6-[2-Chloro-3-(2,4-difluorophenyl)phenyl]-2-imino-6-methyl-3-[(2S*,4S*)-2-methylpiperidin-4-yl]hexahydropyrimidin-4-one

To Example 102 (40.0 mg, 0.08 mmol) was added saturated aqueous NaHCO₃ solution (50 mL) at 0° C. The reaction mixture was stirred for 5 minutes, then extracted with EtOAc (3×25 mL). The organic layer was separated and washed with brine (2×30 mL), then dried over anhydrous sodium sulfate and concentrated in vacuo, to afford the title compound (35.0 mg, 90%) as an off-white solid. LCMS (Method 1, ESI): 446.97 [MH]⁺, RT 0.23 minutes and 446.97 [MH]⁺, RT 0.72 minutes.

### Intermediate 80

### (6S)-6-1[2-Chloro-3-(2,4-difluorophenylphenyl]-3-[(2S*,4S*)-1,2-dimethylpiperidin-4-yl]-2-imino-6-methylhexahydropyrimidin-4-one

To a solution of Intermediate 79 (32.0 mg, 0.07 mmol) and formaldehyde (4.0 mg, 0.13 mmol) in acetic acid (1.5 mL) was added NaBH₃CN (8.0 mg, 0.13 mmol). The reaction mixture was stirred at r.t. for 16 h, then concentrated in vacuo. The residue was poured into water (30 mL), basified with aqueous NaHCO₃ solution (15 mL) and extracted with EtOAc (2×30 mL). The organic layer was separated and washed with brine (2×30 mL), then dried over anhydrous sodium sulfate and concentrated in vacuo. The crude residue was purified by preparative HPLC to afford the title compound (23 mg, 70%) as an off-white solid. δ_{H} (400 MHz, DMSO-d₆) 7.31-7.47 (m, 3H), 7.21-7.30 (m, 1H), 7.11 (d, J 2.93 Hz, 1H), 5.79-6.09 (m, 1H), 2.80-2.92 (m, 1H), 2.63-2.75 (m, 1H), 2.14-2.39 (m, 2H), 2.09 (s, 3H), 1.72-1.97 (m, 2H), 1.57 (s, 3H), 0.97 (d, J 5.87 Hz, 3H). Two exchangeable proton signals not observed; and four proton signals merged into solvent peak.

### Intermediate 81

### tert-Butyl (NE)-N-[(4S)-1-{(1S*,3S*)-3-[tert-butyl(dimethyl)silyl]oxy-4,4-difluoro-cyclohexyl}-4-[2-chloro-3-(2-trimethylsilylethynyl)phenyl]-4-methyl-6-oxohexahydro-pyrimidin-2-ylidene]carbamate

To a solution of Intermediate 39 (100 mg, 0.14 mmol) in THF (5 mL) were added triethylamine (0.06 mL, 0.49 mmol), trimethylsilylacetylene (20.0 mg, 0.25 mmol) and CuI (3.0 mg, 0.014 mmol). The reaction mixture was purged with argon for 10 minutes, then bis(triphenylphosphine)palladium(II) dichloride (10.0 mg, 0.014 mmol) was added. The reaction mixture was stirred at r.t. for 22 h, then concentrated in vacuo. The residue was diluted with water (40 mL) and extracted with EtOAc (2×40 mL). The organic layer was washed with water (150 mL) and brine (150 mL), then separated, dried over anhydrous sodium sulfate and concentrated in vacuo, to afford the crude title compound (90.0 mg) as an off-white solid, which was utilised without further purification. LCMS (Method 1, ESI): 682.3 [MH]⁺, RT 2.98 minutes.

### Intermediate 82

### tert-Butyl (NE)-N-[(4S)-1-{(1S*,3S*)-3-[tert-butyl(dimethyl)silyl]oxy-4,4-difluoro-cyclohexyl}-4-(2-chloro-3-ethynylphenyl)-4-methyl-6-oxohexahydropyrimidin-2-ylidene]carbamate

To a solution of Intermediate 81 (90.0 g, 0.13 mmol) in MeOH (10 mL) was added K₂CO₃ (20.0 mg, 0.13 mmol). The reaction mixture was stirred at r.t. for 4 h, then concentrated in vacuo. The crude residue was purified by flash chromatography (silica, 0-30% EtOAc in hexane) to afford the title compound (70.0 mg, 89%) as an off-white solid. δ_{H} (400 MHz, DMSO-d₆) 10.50 (s, 1H), 7.62 (d, J 1.47 Hz, 1H), 7.35-7.37 (m, 2H), 4.68 (s, 1H), 4.45-4.48 (m, 1H), 3.82-3.85 (m, 1H), 3.56 (d, J 8.56 Hz, 1H), 3.20 (d, J 8.56 Hz, 1H), 2.28-2.31 (m, 2H), 1.90-1.92 (m, 1H), 1.75 (s, 3H), 1.48-1.50 (m, 1H), 1.46 (s, 9H), 1.20-1.22 (m, 1H), 1.01-1.05 (m, 1H), 0.78 (s, 9H), 0.10 (s, 6H). LCMS (Method 1, ESI): 610.27 [MH]⁻, RT 2.70 minutes.

### Intermediate 83

### tert-Butyl (NE)-N-{(4S)-4-[2-chloro-3-(2-trimethylsilylethynyl)phenyl]-4-methyl-1-[(2S,4S)-2-methyltetrahydropyran-4-yl]-6-oxohexahydropyrimidin-2-ylidene}carbamate

Prepared from Intermediate 1 (600.0 mg, 1.07 mmol) in accordance with the procedure described for Intermediate 81 to afford the crude title compound (640.0 mg) as a colourless oil. LCMS (Method 3, ESI): 532.2 [MH]⁺, RT 3.39 minutes.

### Intermediate 84

### tert-Butyl (NE)-N-{(4S)-4-(2-chloro-3-ethynylphenyl)-4-methyl-1-[(2S,4S)-2-methyl-tetrahydropyran-4-yl]-6-oxohexahydropyrimidin-2-ylidene}carbamate

Prepared from Intermediate 83 (640.0 mg, 1.20 mmol) in accordance with the procedure described for Intermediate 82 to afford the title compound (546.0 mg, 99%) as a colourless foam. LCMS (Method 3, ESI): 460.2 [MH]⁺, RT 2.58 minutes.

### Intermediate 85

### tert-Butyl (NE)-N-[(4S)-1-{(1SR,3SR,4RS)-4-[tert-butyl(dimethyl)silyl]oxy-3-methyl-cyclohexyl}-4-[2-chloro-3-(2-trimethylsilylethynyl)phenyl]-4-methyl-6-oxohexahydro-pyrimidin-2-ylidene]carbamate

Prepared from Intermediate 11 (80 mass %) (400.0 mg, 0.46 mmol) in accordance with the procedure described for Intermediate 81 to afford the title compound (320.0 mg, 85%) as a colourless oil. LCMS (Method 3, ESI): 660.4 [MH]⁺, RT 4.42 minutes.

### Intermediate 86

### tert-Butyl (NE)-N-[(4S)-1-{(1SR,3SR,4RS-4-[tert-butyl(dimethyl)silyl]oxy-3-methyl cyclohexyl}-4-(2-chloro-3-ethynylphenyl)-4-methyl-6-oxohexahydropyrimidin-2-ylidene]carbamate

Prepared from Intermediate 85 (320.0 mg, 0.48 mmol) in accordance with the procedure described for Intermediate 82 to afford the title compound (208.0 mg, 73%) as a pale pink oil. LCMS (Method 3, ESI): 588.4 [MH]⁻, RT 3.98 minutes.

### Examples 1 to 129

### General Method 1

To a suspension of the specified aryl iodide derivative (1 equivalent) in 1,4-dioxane (0.1M) and water (1M) were added the specified boronic acid/ester derivative (2-3 equivalents) and Cs₂CO₃ (3 equivalents) at r.t. The reaction mixture was purged with argon for 10 minutes, then Pd(dppf)Cl₂ (0.1 equivalent) was added at r.t. The reaction mixture was purged with nitrogen for 5 minutes, then heated in a sealed tube at 80° C. for 1.5 h, then quenched with water and DCM. The phases were separated, and the aqueous phase was washed with DCM. The combined organic fractions were concentrated in vacuo. The crude residue was purified by column chromatography. To a solution of the resulting material in 1,4-dioxane was added 4M HCl in 1,4-dioxane (5-10 equivalents). The reaction mixture was stirred at r.t. for 4-22 h. The solution was concentrated in vacuo, then lyophilized with acetonitrile/water (5 mL), to afford the title compound (HCl salt). Where the specified aryl iodide derivative contained the tert-butyldimethylsilyloxy moiety, after deprotection the crude material was washed with DME, then lyophilized with acetonitrile/water/aqueous HCl, to afford the title compound (HCl salt).

### General Method 2

To a suspension of the specified aryl iodide derivative (1 equivalent) in 1,4-dioxane (0.1M) and water (1M) were added the specified boronic acid/ester derivative (2-3 equivalents) and Cs₂CO₃ (3 equivalents) at r.t. The reaction mixture was purged with argon for 10 minutes, then Pd(dppf)Cl₂ (0.1 equivalent) was added at r.t. The reaction mixture was purged with nitrogen for 5 minutes, then heated in a sealed tube at 80° C. for 1.5 h, then quenched with water and DCM. The phases were separated, and the aqueous phase was washed with DCM. The combined organic fractions were concentrated in vacuo. The crude residue was purified by chiral separation to obtain two diastereomeric isomers. To a solution of each diastereomer in 1,4-dioxane was added 4M HCl in 1,4-dioxane (5-10 equivalents). The reaction mixture was stirred at r.t. for 4-22 h. The solution was concentrated in vacuo, then lyophilized with acetonitrile/water (5 mL), to afford the title compound (HCl salt). Where the specified aryl iodide derivative contained the tert-butyldimethylsilyloxy moiety, after deprotection the crude material was washed with DME, then lyophilized with acetonitrile/water/aqueous HCl, to afford the title compound (HCl salt).

### General Method 3

A stirred suspension of the specified boronic acid derivative (1 equivalent), the specified aryl iodide derivative (1.1 equivalents) and K₂CO₃ (3 equivalents) in EtOH (4 mL) and water (1 mL) was purged with argon for 30 minutes. XPhos Pd G2 (0.05 equivalent) and XPhos (0.2 equivalent) were added at r.t. The reaction mixture was heated in a sealed tube at 80° C. for 1 h, then concentrated in vacuo. The residue was diluted with brine (50 mL) and extracted with EtOAc (2×100 mL). The organic layer was separated, dried over anhydrous sodium sulfate and concentrated in vacuo. The crude residue was purified by column chromatography. The resulting solid was re-dissolved in DCM (5 mL), and TFA (20 equivalents) was added at 0° C. The reaction mixture was stirred at r.t. for 6 h, then concentrated in vacuo. The resulting crude material was purified by washing with diethyl ether (5 mL) and hexane (10 mL), then lyophilized with acetonitrile/water (5 mL), to afford the title compound (TFA salt).

### General Method 4

A stirred suspension of the specified boronic acid derivative (1 equivalent), the specified aryl iodide derivative (1 equivalent) and K₃PO₄ (3 equivalents) in 1,4-dioxane (6 mL) and water (2 mL) was purged with argon for 30 minutes. Pd(dppf)Cl₂ (0.13 equivalent) was added at r.t. The reaction mixture was heated in sealed tube at 80° C. for 1.5 h, then concentrated in vacuo. The residue was diluted with brine (50 mL) and extracted with EtOAc (2×100 mL). The organic layer was separated, dried over anhydrous sodium sulfate and concentrated in vacuo. The crude residue was purified by column chromatography. The resulting solid was re-dissolved in DCM (5 mL), and TFA (20 equivalents) was added at 0° C. The reaction mixture was stirred at r.t. for 16 h, then concentrated in vacuo. The resulting crude material was purified by washing with diethyl ether (5 mL) and hexane (10 mL), then lyophilized with acetonitrile/water (5 mL), to afford the title compound (TFA salt).

### General Method 5

To a solution of the specified Intermediate (1 equivalent) in DCM (5 mL) was added TFA (1 equivalent) at 0° C. over 10 minutes. The reaction mixture was stirred at r.t. for 3 h, then concentrated in vacuo. The crude residue was purified by triturating with diethyl ether (10 mL), then dried in vacuo, to afford the title compound (TFA salt).

### General Method 6

A solution of the specified Intermediate (1 equivalent) in HCl (2M in 1,4-dioxane) (80 equivalents) was stirred at r.t. for 4 h. The resulting solution was concentrated in vacuo, then lyophilized with acetonitrile/water (5 mL), to afford the title compound (HCl salt).

### General Method 7

To a mixture of potassium carbonate (3.0 equivalents) and the specified boronic acid/ester derivative (1.3 equivalents) were added a degassed solution of the specified aryl iodide derivative (1.0 equivalent), XPhos Pd G3 (0.1 equivalent) and XPhos (0.1 equivalent) in 1,4-dioxane/water (4:1) (0.5 mL). The mixture was heated at 90-100° C. for 1.5 h, then filtered. Acetonitrile (0.5 mL) was added, then the mixture was purified by preparative HPLC. To the resulting material was added TFA/DCM (1 mL), and the mixture was stirred at r.t. for 1 h, then concentrated in vacuo. Purification by preparative HPLC afforded the title compound (TFA salt).

### General Method 8

To a suspension of the specified aryl iodide derivative (1 equivalent) in 1,4-dioxane (0.1M) and water (1M) were added the specified boronic acid/ester derivative (2-3 equivalents) and Cs₂CO₃ (3 equivalents) at r.t. The reaction mixture was purged with argon for 10 minutes, then Pd(dppf)Cl₂ (0.1 equivalent) was added at r.t. The reaction mixture was purged with nitrogen for 5 minutes, then heated in a sealed tube at 80° C. for 1.5 h, then quenched with water and DCM. The phases were separated, and the aqueous phase was washed with DCM. The combined organic fractions were concentrated in vacuo. The crude residue was purified by chiral HPLC to afford two, three or four diastereomers. To a solution of each separated diastereomer in 1,4-dioxane was added 4M HCl in 1,4-dioxane (5-10 equivalents). The reaction mixture was stirred at r.t. for 16 h. The solution was concentrated in vacuo, then lyophilized with acetonitrile/water (5 mL), to afford the title compound (HCl salt).

### General Method 9

To a suspension of the specified aryl iodide derivative (1 equivalent) in 1,4-dioxane (0.1M) and water (1M) were added the specified boronic acid/ester derivative (2-3 equivalents) and Cs₂CO₃ (3 equivalents) at r.t. The reaction mixture was purged with argon for 10 minutes, then Pd(dppf)Cl₂ (0.1 equivalent) was added at r.t. The reaction mixture was purged with nitrogen for 5 minutes, then heated in a sealed tube at 80° C. for 1.5 h, then quenched with water and DCM. The phases were separated, and the aqueous phase was washed with DCM. The combined organic fractions were concentrated in vacuo. The crude material was re-dissolved in DCM, and TFA was added. The mixture was stirred at r.t. for 1-2 h, then concentrated in vacuo. The crude residue was purified by chiral/achiral HPLC to afford the title compound (free base).

### General Method 10

To a solution of the specified amino ester derivative (1.0 equivalent), the specified activated thioester derivative (1.2 equivalents) and EDC·HCl (1.5 equivalents) in DMF (0.5M) at 0° C. was added DIPEA (2.0 equivalents). The reaction mixture was stirred at r.t. for 4 h, then diluted with ice-cold water and extracted with EtOAc. The organic layer was separated and washed with brine, then dried over anhydrous sodium sulfate and concentrated in vacuo. The resulting crude material was dissolved in THF (0.1M), and potassium tert-butoxide (1M in THF, 1.3 equivalents) was added at 0° C. The reaction mixture was stirred at r.t. for 2 h, then quenched with water and extracted with EtOAc. The organic layer was separated, dried over anhydrous sodium sulfate and concentrated in vacuo. The crude residue was purified by chiral preparative HPLC (Column: Lux Cellulose-1 150×4.6 mm, 3 μm; Column Temperature: 35° C.; Flow Rate: 3 mL/minute; Method: 3-40% MeOH+0.1% NH₄OH over 6.5 minutes) to afford separated diastereomers. To a solution of each separated diastereomer in 1,4-dioxane (0.1M) was added 4M HCl in 1,4-dioxane (5 equivalents). The reaction mixture was stirred at r.t. for 18 h. The solution was concentrated in vacuo, then lyophilized with acetonitrile/water (5 mE), to afford the title compound (HCl salt).

### General Method 11

To a solution of the specified Intermediate (0.04 mmol) in DCM (2 mL) was added 4M HCl in 1,4-dioxane (0.08 mmol) at 0° C. The mixture was stirred at r.t. for 30 minutes, then concentrated in vacuo. The crude residue was washed with a mixture of diethyl ether (2 mL) and n-pentane (5 mL), then dried, to afford the title compound (HCl salt).

### General Method 12

To a solution of the specified aniline derivative (5.0 equivalents) in acetonitrile (0.25M) were added 2-azido-1,3-dimethylimidazolinium hexafluorophosphate (7.7 equivalents) and DMAP (15.6 equivalents) at r.t. The reaction mixture was stirred for 21 h at 50° C., then the specified aryl alkyne derivative (1.0 equivalent), sodium ascorbate (4.5 equivalents), cupric sulfate (1.6 equivalents) and water (0.05M) were added. The reaction mixture was stirred at r.t. for 2 h. The aqueous layer was extracted with EtOAc. The organic layer was separated and dried over anhydrous sodium sulfate, then concentrated in vacuo. The crude residue was purified by column chromatography. The resulting material was dissolved in 4M HCl in 1,4-dioxane (5-10 equivalents). The reaction mixture was stirred at r.t. for 17 h, then concentrated in vacuo. The crude residue was dissolved in MeOH and purified by SCX-2 (1 g cartridge). The resulting material was lyophilized with acetonitrile/water/0.5N HCl to afford the title compound (HCl salt).

### General Method 13

To the specified aryl alkyne derivative (1.0 equivalent) and the specified azide derivative (1.0 equivalent) in tert-butanol (0.5 mL) was added a solution of sodium ascorbate (0.4 equivalent) and copper(II) sulfate pentahydrate (0.4 equivalent) in water (0.5 mL) at r.t. The mixture was stirred at 40° C. for 16 h, then filtered, rinsed with acetonitrile/water (7:3) (0.5 mL) and concentrated in vacuo. The crude material was purified by preparative HPLC. To the resulting purified material was added TFA/DCM (1 mL), and the mixture was stirred at r.t. for 1 h, then concentrated in vacuo and purified by preparative HPLC, to afford the title compound (TFA salt).

### Examples 1 to 129

Example 1 was prepared from Intermediate 1 and 5-chloropyridine-3-boronic acid in accordance with General Method 1.

Example 2 was prepared from Intermediate 1 and 2-chloro-4-(trifluoromethoxy)-boronic acid in accordance with General Method 1.

Example 3 was prepared fromIntermediate 1 and (dimethyl)(oxo)[3-(4,4,5,5-tetramethyl-1,3,2-dioxaborolan-2-yl)phenyl]imino-k⁶-sulfane in accordance with General Method 1.

Example 4 was prepared from Intermediate 1 and 3-(methylsulfonyl)phenyl-boronic acid in accordance with General Method 1.

Example 5 was prepared from Intermediate 1 and 2-[3-(dimethylphosphoryl)-phenyl]-4,4,5,5-tetramethyl-1,3,2-dioxaborolane in accordance with General Method 1.

Example 6 was prepared from Intermediate 1 and Intermediate 3 in accordance with General Method 1.

Example 7 and Example 8 were prepared from Intermediate 11 and (3-chloro-phenyl)boronic acid in accordance with General Method 2.

Example 9 and Example 10 were prepared from Intermediate 26 and Intermediate 30 in accordance with General Method 2.

Example 11 and Example 12 were prepared from Intermediate 26 and Intermediate 28 in accordance with General Method 2.

Example 13 was prepared from Intermediate 31 and 3-(methylsulfonylamino)-phenylboronic acid in accordance with General Method 3.

Example 14 was prepared from Intermediate 31 and 4-(methylsulfonylamino)-phenylboronic acid in accordance with General Method 3.

Example 15 was prepared from Intermediate 1 and 4-chloro-2-fluorophenyl-boronic acid in accordance with General Method 3.

Example 16 was prepared from Intermediate 1 and 2,4-dichlorophenylboronic acid in accordance with General Method 3.

Example 17 was prepared from Intermediate 1 and 3-chlorophenylboronic acid in accordance with General Method 3.

Example 18 was prepared from Intermediate 31 and Intermediate 28 in accordance with General Method 1.

Example 19 was prepared from Intermediate 31 and Intermediate 33 in accordance with General Method 1.

Example 20 was prepared from Intermediate 31 and (dimethyl)(oxo)[3-(4,4,5,5-tetramethyl-1,3,2-dioxaborolan-2-yl)phenyl]imino-λ⁶-sulfane in accordance with General Method 1.

Example 21 was prepared from Intermediate 39 and (3-chlorophenyl)boronic acid in accordance with General Method 4.

Example 22 was prepared from Intermediate 41 and (3-chlorophenyl)boronic acid in accordance with General Method 4.

Example 23 was prepared from Intermediate 41 and (5-chloropyridin-3-yl)boronic acid in accordance with General Method 1.

Example 24 was prepared from Intermediate 45 in accordance with General Method 5.

Example 25 was prepared from Intermediate 48 in accordance with General Method 6.

Example 26 was prepared from Intermediate 49 in accordance with General Method 6.

Example 27 was prepared from Intermediate 51 in accordance with General Method 6.

Example 28 was prepared from Intermediate 52 in accordance with General Method 6.

Example 29 was prepared from Intermediate 1 and 4-[3-(4,4,5,5-tetramethyl-1,3,2-dioxaborolan-2-yl)phenyl]morpholine in accordance with General Method 7.

Example 30 was prepared from Intermediate 1 and 1-methyl-2-(4,4,5,5-tetramethyl-1,3,2-dioxaborolan-2-yl)-1H-pyrrolo[2,3-b]pyridine in accordance with General Method 7.

Example 31 was prepared from Intermediate 1 and 6-cyclopropylpyridin-3-ylboronic acid in accordance with General Method 7.

Example 32 was prepared from Intermediate 1 and 3-methyl-5-(4,4,5,5-tetramethyl-1,3,2-dioxaborolan-2-yl)isoxazole in accordance with General Method 7.

Example 33 was prepared from Intermediate 1 and 4-(4,4,5,5-tetramethyl-1,3,2-dioxaborolan-2-yl)pyridazine in accordance with General Method 7.

Example 34 was prepared from Intermediate 1 and 1-methyl-4-(4,4,5,5-tetramethyl-1,3,2-dioxaborolan-2-yl)pyridin-2(1H)-one in accordance with General Method 7.

Example 35 was prepared from Intermediate 1 and 4,4,5,5-tetramethyl-2-(naphthalen-1-yl)-1,3,2-dioxaborolane in accordance with General Method 7.

Example 36 was prepared from Intermediate 1 and 2-(2-isopropylphenyl)-4,4,5,5-tetramethyl-1,3,2-dioxaborolane in accordance with General Method 7.

Example 37 was prepared from Intermediate 1 and 2-(trifluoromethyl)phenyl-boronic acid in accordance with General Method 7.

Example 38 was prepared from Intermediate 1 and 3,4-difluorophenylboronic acid in accordance with General Method 7.

Example 39 was prepared from Intermediate 1 and pyridine-4-boronic acid in accordance with General Method 7.

Example 40 was prepared from Intermediate 1 and 3-cyanophenylboronic acid in accordance with General Method 7.

Example 41 was prepared from Intermediate 1 and pyrimidine-5-boronic acid in accordance with General Method 7.

Example 42 was prepared from Intermediate 1 and 2,5-difluorophenylboronic acid in accordance with General Method 7.

Example 43 was prepared from Intermediate 1 and 2-methoxy-5-pyridineboronic acid in accordance with General Method 7.

Example 44 was prepared from Intermediate 1 and 3,5-difluorophenylboronic acid in accordance with General Method 7.

Example 45 was prepared from Intermediate 1 and (1H-pyrazol-5-yl)boronic acid hydrate in accordance with General Method 7.

Example 46 was prepared from Intermediate 1 and 2-chlorophenylboronic acid in accordance with General Method 7.

Example 47 was prepared from Intermediate 1 and 2,6-difluorophenylboronic acid in accordance with General Method 7.

Example 48 was prepared from Intermediate 1 and 3-(trifluoromethyl)phenyl-boronic acid in accordance with General Method 7.

Example 49 was prepared from Intermediate 1 and pyridine-3-boronic acid in accordance with General Method 7.

Example 50 was prepared from Intermediate 1 and 2,3-difluorophenylboronic acid in accordance with General Method 7.

Example 51 was prepared from Intermediate 1 and 2,4-difluorophenylboronic acid in accordance with General Method 7.

Example 52 was prepared from Intermediate 1 and 1,3-dimethyl-5-(4,4,5,5-tetramethyl-1,3,2-dioxaborolan-2-yl)pyrazole in accordance with General Method 7.

Example 53 was prepared from Intermediate 1 and [3-(1H-pyrazol-5-yl)phenyl]-boronic acid hydrate in accordance with General Method 7.

Example 54 was prepared from Intermediate 1 and 4-isoxazoleboronic acid pinacol ester in accordance with General Method 7.

Example 55 was prepared from Intermediate 1 and (6-chloropyridin-3-yl)boronic acid in accordance with General Method 7.

Example 56 was prepared from Intermediate 1 and 1-methylindazole-7-boronic acid in accordance with General Method 7.

Example 57 was prepared from Intermediate 1 and 3-chloro-4-pyridineboronic acid in accordance with General Method 7.

Example 58 was prepared from Intermediate 1 and 5-methoxypyridine-3-boronic acid in accordance with General Method 7.

Example 59 was prepared from Intermediate 1 and 2-methoxypyridine-4-boronic acid in accordance with General Method 7.

Example 60 was prepared from Intermediate 1 and 4-cyanophenylboronic acid in accordance with General Method 7.

Example 61 was prepared from Intermediate 1 and 2-chloropyridine-3-boronic acid in accordance with General Method 7.

Example 62 was prepared from Intermediate 1 and pyrazolo[1,5-a]pyrimidine-3-boronic acid pinacol ester in accordance with General Method 7.

Example 63, Example 64, Example 65 and Example 66 were prepared from Intermediate 58 and 2,4-difluorophenylboronic acid in accordance with General Method 8.

Example 67, Example 68 and Example 69 were prepared from Intermediate 58 and 6-cyclopropypyridin-3-ylboronic acid in accordance with General Method 8.

Example 70, Example 71, Example 72 and Example 73 were prepared from Intermediate 58 and 2-(difluoromethoxy)-5-(4,4,5,5-tetramethyl-1,3,2-dioxaborolan-2-yl)pyridine in accordance with General Method 8.

Example 74, Example 75, Example 76 and Example 77 were prepared from Intermediate 58 and 5-chloropyridine-2-boronic acid in accordance with General Method 8.

Example 78, Example 79, Example 80 and Example 81 were prepared from Intermediate 58 and [6-(trifluoromethoxy)pyridin-3-yl]boronic acid in accordance with General Method 8.

Example 82 and Example 83 were prepared from Intermediate 59 and 5-chloro-pyridine-3-boronic acid in accordance with General Method 8.

Example 84 and Example 85 were prepared from Intermediate 60 and 4-(4,4,5,5-tetramethyl-1,3,2-dioxaborolan-2-yl)-1-(2,2,2-trifluoroethyl)-1H-pyrazole in accordance with General Method 8.

Example 86 and Example 87 were prepared from Intermediate 60 and 3-cyclopropyl-5-(4,4,5,5-tetramethyl-1,3,2-dioxaborolan-2-yl)isoxazole in accordance with General Method 8.

Example 88 and Example 89 were prepared from Intermediate 62 and 2,4-difluorophenylboronic acid in accordance with General Method 9.

Example 90 was prepared from Intermediate 64 and 3-chlorophenylboronic acid in accordance with General Method 9.

Example 91 was prepared from Intermediate 64 and 2-chloro-4-(trifluoro-methoxy)phenylboronic acid in accordance with General Method 9.

Example 92 was prepared from Intermediate 64 and 2,5-dichlorophenylboronic acid in accordance with General Method 9.

Example 93 was prepared from Intermediate 64 and 2,4-difluorophenylboronic acid in accordance with General Method 9.

Example 94 was prepared from Intermediate 66 and Intermediate 72 in accordance with General Method 10.

Example 95 and Example 96 were prepared from Intermediate 66 and Intermediate 74 in accordance with General Method 10.

Example 97 was prepared from Intermediate 68 and Intermediate 22 in accordance with General Method 10.

Example 98 and Example 99 were prepared from Intermediate 70 and Intermediate 22 in accordance with General Method 10.

Example 100 and Example 101 were prepared from Intermediate 66 and Intermediate 75 in accordance with General Method 10.

Example 102 and Example 103 were prepared from Intermediate 68 and Intermediate 75 in accordance with General Method 10.

Example 104 and Example 105 were prepared from Intermediate 66 and Intermediate 78 in accordance with General Method 10.

Example 106 was prepared from Intermediate 80 in accordance with General Method 11.

Example 107 was prepared from Example 103 in accordance with the procedure described for the preparation of Example 106 from Example 102.

Example 108 was prepared from Intermediate 82 and 3-fluoroaniline in accordance with General Method 12.

Example 109 was prepared from Intermediate 84 and 2,4,5-trifluoroaniline in accordance with General Method 12.

Example 110 was prepared from Intermediate 84 and 2-azido-1,1,1-trifluoro-ethane in accordance with General Method 12, omitting the first step.

Example 111 was prepared from Intermediate 84 and 3-fluoroaniline in accordance with General Method 12.

Example 112 and Example 113 were prepared from Intermediate 86 and 2,4,5-trifluoroaniline in accordance with General Method 12.

Example 114 was prepared from Intermediate 84 and (2-azidoethyl)dimethyl-amine hydrochloride in accordance with General Method 13.

Example 115 was prepared from Intermediate 84 and 3-[(1S)-1-azidoethyl]-pyridine in accordance with General Method 13.

Example 116 was prepared from Intermediate 84 and 3-(azidomethyl)-1,1-difluorocyclobutane in accordance with General Method 13.

Example 117 was prepared from Intermediate 84 and 3-(azidomethyl)pyridine in accordance with General Method 13.

Example 118 was prepared from Intermediate 84 and 1-(2-azidoethyl)pyrrolidine in accordance with General Method 13.

Example 119 was prepared from Intermediate 84 and 4-azidopyridine in accordance with General Method 13.

Example 120 was prepared from Intermediate 84 and 4-(azidomethyl)pyridine in accordance with General Method 13.

Example 121 was prepared from Intermediate 84 and (1-azidoethenyl)benzene in accordance with General Method 13.

Example 122 was prepared from Intermediate 84 and azidocyclobutane in accordance with General Method 13.

Example 123 was prepared from Intermediate 84 and 1-azido-2-methoxyethane in accordance with General Method 13.

Example 124 was prepared from Intermediate 84 and benzyl azide in accordance with General Method 13.

Example 125 was prepared from Intermediate 84 and 1-(1-azidoethyl)-2-oxabicyclo[2.1.1]hexane in accordance with General Method 13.

Example 126 was prepared from Intermediate 84 and azidobenzene in accordance with General Method 13.

Example 127 was prepared from Intermediate 84 and 1-azidobutane in accordance with General Method 13.

Example 128 was prepared from Intermediate 84 and 3-[(1R)-1-azidoethyl]-pyridine in accordance with General Method 13.

Example 129 was prepared from Intermediate 84 and 4-azidoazepane in accordance with General Method 13.

| | | | LCMS* | |
|---|---|---|---|---|
| Ex. | Product | Structure | RT (min) | [MH]⁺ |
| 1 | (6S)-6-[2-Chloro-3-(5-chloro- pyridin-3-yl)phenyl]-2-imino-6- methyl-3-[(2S,4S)-2-methyl- tetrahydropyran-4-yl]hexahydro- pyrimidin-4-one hydrochloride | 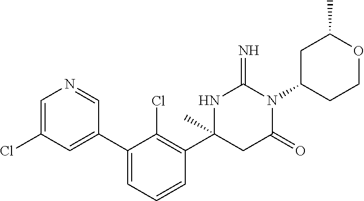 [SMILES: C[C@H]1C[C@@H](N2C(=N)N[C@](C)(c3cccc(-c4cncc(Cl)c4)c3Cl)CC2=O)CCO1] | 0.75 | 447.3 |
| 2 | (6S)-6-{2-Chloro-3-[2-chloro-4- (trifluoromethoxy)phenyl]phenyl}- 2-imino-6-methyl-3-[(2S,4S)-2- methyltetrahydropyran-4-yl]- hexahydropyrimidin-4-one hydrochloride | 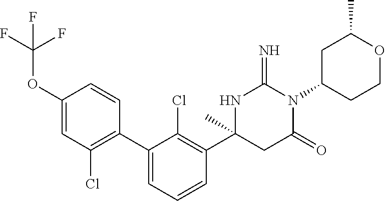 [SMILES: C[C@H]1C[C@@H](N2C(=N)N[C@](C)(c3cccc(-c4ccc(OC(F)(F)F)cc4Cl)c3Cl)CC2=O)CCO1] | 2.22 | 530.2 |
| 3 | (6S)-6-[2-Chloro-3-(3-{[(dimethyl)- (oxo)-26-sulfanylidene]amino}- phenyl)phenyl]-2-imino-6-methyl- 3-[(2S,4S)-2-methyltetrahydro- pyran-4-yl]hexahydropyrimidin-4- one hydrochloride | 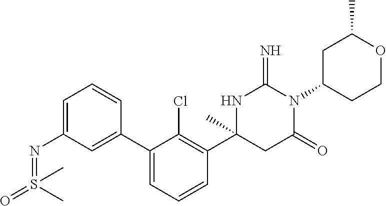 [SMILES: C[C@H]1C[C@@H](N2C(=N)N[C@](C)(c3cccc(-c4cccc(N=S(C)(C)=O)c4)c3Cl)CC2=O)CCO1] | 0.67 | 503.4 |
| 4 | (6S)-6-[2-Chloro-3-(3-methyl- sulfonylphenyl)phenyl]-2-imino-6- methyl-3-[(2S,4S)-2-methyl- tetrahydropyran-4-yl]hexahydro- pyrimidin-4-one hydrochloride | 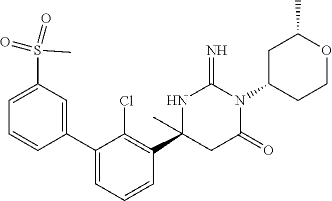 [SMILES: C[C@H]1C[C@@H](N2C(=N)N[C@](C)(c3cccc(-c4cccc(S(C)(=O)=O)c4)c3Cl)CC2=O)CCO1] | 0.68 | 490.4 |
| 5 | (6S)-6-[2-Chloro-3-(3-dimethyl- phosphorylphenyl)phenyl]-2-imino- 6-methyl-3-[(2S,4S)-2-methyl- tetrahydropyran-4-yl]hexahydro- pyrimidin-4-one hydrochloride | 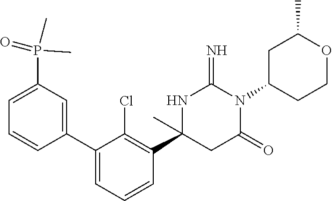 [SMILES: C[C@H]1C[C@@H](N2C(=N)N[C@](C)(c3cccc(-c4cccc(P(C)(C)=O)c4)c3Cl)CC2=O)CCO1] | 1.21 | 488.2 |
| 6 | (6S)-6-{2-Chloro-3-[3-(2,2,2- trifluoroethyl)benzimidazol-5-yl]- phenyl}-2-imino-6-methyl-3- [(2S,4S)-2-methyltetrahydropyran- 4-yl]hexahydropyrimidin-4-one hydrochloride | 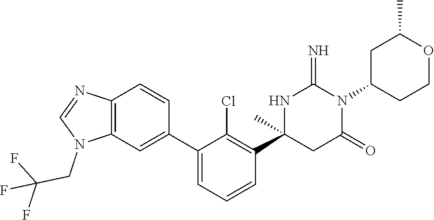 [SMILES: C[C@H]1C[C@@H](N2C(=N)N[C@](C)(c3cccc(-c4ccc5ncn(CC(F)(F)F)c5c4)c3Cl)CC2=O)CCO1] | 0.75 | 534.4 |
| 7 | (6S)-6-[2-Chloro-3-(3-chloro- phenyl)phenyl]-3-[(1R*,3R*,4S*)- 4-hydroxy-3-methylcyclohexyl]-2- imino-6-methylhexahydro- pyrimidin-4-one hydrochloride | 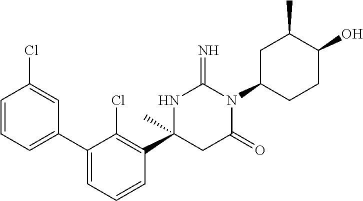 [SMILES: C[C@@H]1C[C@H](N2C(=N)N[C@](C)(c3cccc(-c4cccc(Cl)c4)c3Cl)CC2=O)CC[C@@H]1O] | 0.92 | 460.4 |
| 8 | (6S)-6-[2-Chloro-3-(3-chloro- phenyl)phenyl]-3-[(1S*,3S*,4R*)- 4-hydroxy-3-methylcyclohexyl]-2- imino-6-methylhexahydro- pyrimidin-4-one hydrochloride | 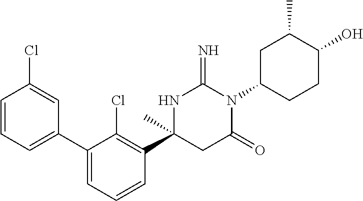 [SMILES: C[C@H]1C[C@@H](N2C(=N)N[C@](C)(c3cccc(-c4cccc(Cl)c4)c3Cl)CC2=O)CC[C@H]1O] | 0.92 | 460.4 |
| 9 | (6S)-6-{2-Chloro-3-[2-fluoro-4- (methylsulfonyl)phenyl]phenyl}-3- [(1R*,3R*)-4,4-difluoro-3- methoxycyclohexyl]-2-imino-6- methylhexahydropyrimidin-4-one hydrochloride | 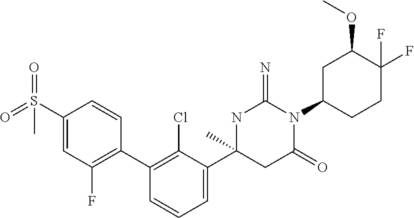 [SMILES: CO[C@@H]1C[C@H](N2C(=N)N[C@](C)(c3cccc(-c4ccc(S(C)(=O)=O)cc4F)c3Cl)CC2=O)CCC1(F)F] | 0.74 | 558.4 |
| 10 | (6S)-6-{2-Chloro-3-[2-fluoro-4- (methylsulfonyl)phenyl]phenyl}-3- [(1S*,3S*)-4,4-difluoro-3-methoxy- cyclohexyl]-2-imino-6-methyl- hexahydropyrimidin-4-one hydrochloride | 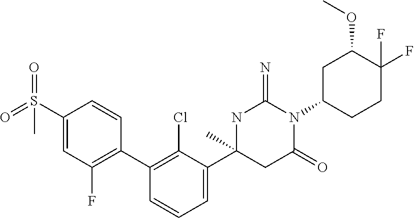 [SMILES: CO[C@H]1C[C@@H](N2C(=N)N[C@](C)(c3cccc(-c4ccc(S(C)(=O)=O)cc4F)c3Cl)CC2=O)CCC1(F)F] | 0.77 | 558.4 |
| 11 | (6S)-6-[2-Chloro-3-(4-{[(dimethyl)- (oxo)-λ⁶-sulfanylidene]amino}-2- fluorophenyl)phenyl]-3- [(1R*,3R*)-4,4-difluoro-3- methoxycyclohexyl]-2-imino-6- methylhexahydropyrimidin-4-one hydrochloride | 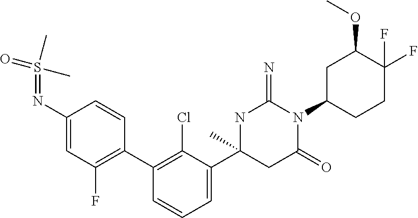 [SMILES: CO[C@@H]1C[C@H](N2C(=N)N[C@](C)(c3cccc(-c4ccc(N=S(C)(C)=O)cc4F)c3Cl)CC2=O)CCC1(F)F] | 0.73 | 571.4 |
| 12 | (6S)-6-[2-Chloro-3-(4-{[(dimethyl)- (oxo)-λ⁶-sulfanylidene]amino}-2- fluorophenyl)phenyl]-3-[(1S*,3S*)- 4,4-difluoro-3-methoxy- cyclohexyl]-2-imino-6-methyl- hexahydropyrimidin-4-one hydrochloride | 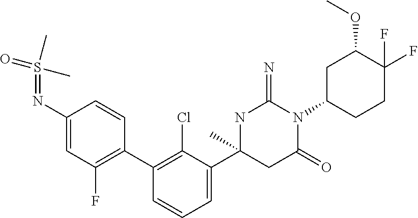 [SMILES: CO[C@H]1C[C@@H](N2C(=N)N[C@](C)(c3cccc(-c4ccc(N=S(C)(C)=O)cc4F)c3Cl)CC2=O)CCC1(F)F] | 0.75 | 571.4 |
| 13 | N-[3-(2-Chloro-3-{(4S)-1-[(1S,3S)- 4,4-difluoro-3-methylcyclohexyl]- 2-imino-4-methyl-6-oxohexahydro- pyrimidin-4-yl}phenyl)phenyl]- methanesulfonamide trifluoroacetic acid salt | 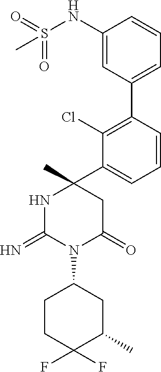 [SMILES: C[C@H]1C[C@@H](N2C(=N)N[C@](C)(c3cccc(-c4cccc(NS(C)(=O)=O)c4)c3Cl)CC2=O)CCC1(F)F] | 0.87 | 539.3 |
| 14 | N-[4-(2-Chloro-3-{(4S)-1-[(1S,3S)- 4,4-difluoro-3-methylcyclohexyl]- 2-imino-4-methyl-6-oxohexahydro- pyrimidin-4-yl}phenyl)phenyl]- methanesulfonamide trifluoroacetic acid salt | 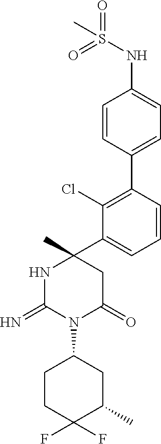 [SMILES: C[C@H]1C[C@@H](N2C(=N)N[C@](C)(c3cccc(-c4ccc(NS(C)(=O)=O)cc4)c3Cl)CC2=O)CCC1(F)F] | 0.85 | 539.3 |
| 15 | (6S)-6-[2-Chloro-3-(4-chloro-2- fluorophenyl)phenyl]-2-imino-6- methyl-3-[(2S,4S)-2-methyl- tetrahydropyran-4-yl]hexahydro- pyrimidin-4-one trifluoroacetic acid salt | 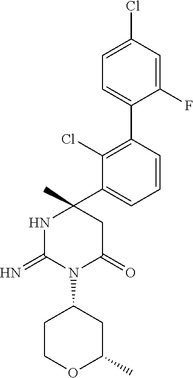 [SMILES: C[C@H]1C[C@@H](N2C(=N)N[C@](C)(c3cccc(-c4ccc(Cl)cc4F)c3Cl)CC2=O)CCO1] | 0.94 | 464.4 |
| 16 | (6S)-6-[2-Chloro-3-(2,4-dichloro- phenyl)phenyl]-2-imino-6-methyl- 3-[(2S,4S)-2-methyltetrahydro- pyran-4-yl]hexahydropyrimidin-4- one trifluoroacetic acid salt | 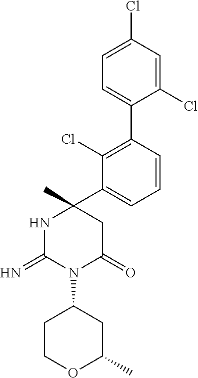 [SMILES: C[C@H]1C[C@@H](N2C(=N)N[C@](C)(c3cccc(-c4ccc(Cl)cc4Cl)c3Cl)CC2=O)CCO1] | 0.98 | 482.2 |
| 17 | (6S)-6-[2-Chloro-3-(3-chloro- phenyl)phenyl]-2-imino-6-methyl- 3-[(2S,4S)-2-methyltetrahydro- pyran-4-yl]hexahydropyrimidin-4- one trifluoroacetic acid salt | 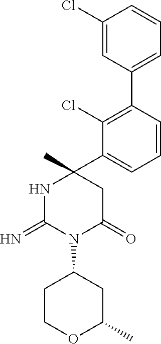 [SMILES: C[C@H]1C[C@@H](N2C(=N)N[C@](C)(c3cccc(-c4cccc(Cl)c4)c3Cl)CC2=O)CCO1] | 0.93 | 446.4 |
| 18 | (6S)-6-[2-Chloro-3-(4-{[(dimethyl)- (oxo)-λ⁶-sulfanylidene]amino}-2- fluorophenyl)phenyl]-3-[(1S,3S)- 4,4-difluoro-3-methylcyclohexyl]- 2-imino-6-methylhexahydro- pyrimidin-4-one hydrochloride | 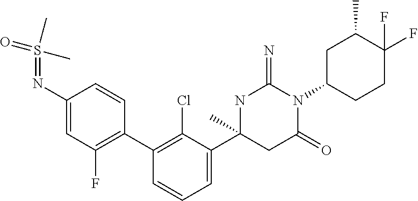 [SMILES: C[C@H]1C[C@@H](N2C(=N)N[C@](C)(c3cccc(-c4ccc(N=S(C)(C)=O)cc4F)c3Cl)CC2=O)CCC1(F)F] | 0.84 | 555.4 |
| 19 | (6S)-6-[2-Chloro-3-(4-dimethyl- phosphoryl-2-fluorophenyl)- phenyl]-3-[(1S,3S)-4,4-difluoro-3- methylcyclohexyl]-2-imino-6- methylhexahydropyrimidin-4-one hydrochloride | 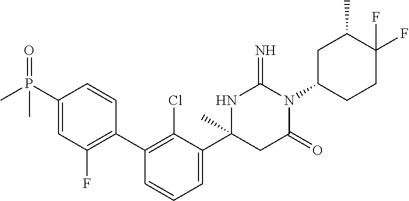 [SMILES: C[C@H]1C[C@@H](N2C(=N)N[C@](C)(c3cccc(-c4ccc(P(C)(C)=O)cc4F)c3Cl)CC2=O)CCC1(F)F] | 0.77 | 540.3 |
| 20 | (6S)-6-[2-Chloro-3-(3-{[(dimethyl)- (oxo)-λ⁶-sulfanylidene]amino}- phenyl)phenyl]-3-[(1S,3S)-4,4- difluoro-3-methylcyclohexyl]-2- imino-6-methylhexahydro- pyrimidin-4-one hydrochloride | 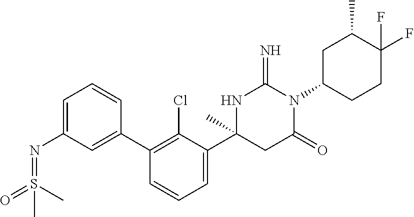 [SMILES: C[C@H]1C[C@@H](N2C(=N)N[C@](C)(c3cccc(-c4cccc(N=S(C)(C)=O)c4)c3Cl)CC2=O)CCC1(F)F] | 0.84 | 537.4 |
| 21 | (S)-6-[2,3′-Dichloro(1,1′-biphenyl)- 3-yl]-3-[(1S*,3S*)-4,4-difluoro-3- hydroxycyclohexyl]-2-imino-6- methyltetrahydropyrimidin-4(1H)- one trifluoroacetic acid salt | 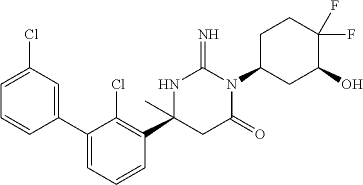 [SMILES: C[C@@]1(c2cccc(-c3cccc(Cl)c3)c2Cl)CC(=O)N([C@H]2CCC(F)(F)[C@@H](O)C2)C(=N)N1] | 0.92 | 482.3 |
| 22 | (S)-6-[2,3′-Dichloro(1,1′-biphenyl)- 3-yl]-3-[(1R*,3R*)-4,4-difluoro-3- hydroxycyclohexyl]-2-imino-6- methyltetrahydropyrimidin-4(1H)- one trifluoroacetic acid salt | 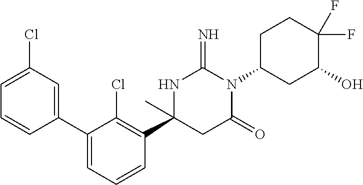 [SMILES: C[C@@]1(c2cccc(-c3cccc(Cl)c3)c2Cl)CC(=O)N([C@@H]2CCC(F)(F)[C@H](O)C2)C(=N)N1] | 0.85 | 482.3 |
| 23 | (6S)-6-[2-Chloro-3-(5-chloro- pyridin-3-yl)phenyl]-3-[(1R*,3R*)- 4,4-difluoro-3-hydroxycyclohexyl]- 2-imino-6-methylhexahydro- pyrimidin-4-one hydrochloride | 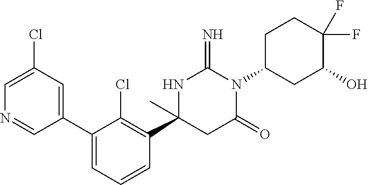 [SMILES: C[C@@]1(c2cccc(-c3cncc(Cl)c3)c2Cl)CC(=O)N([C@@H]2CCC(F)(F)[C@H](O)C2)C(=N)N1] | 0.67 | 483.3 |
| 24 | (6S)-6-[2-Chloro-3-(3-chloro- phenyl)phenyl]-3-[(1R*,3S*)-4,4- difluoro-3-hydroxycyclohexyl]-2- imino-6-methylhexahydro- pyrimidin-4-one trifluoroacetic acid salt | 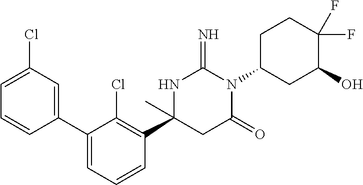 [SMILES: C[C@@]1(c2cccc(-c3cccc(Cl)c3)c2Cl)CC(=O)N([C@@H]2CCC(F)(F)[C@@H](O)C2)C(=N)N1] | 0.84 | 482.4 |
| 25 | (6S)-6-(2-Chloro-3-{3-[(1R*)-2,2- difluoro-1-hydroxyethyl]phenyl}- phenyl)-2-imino-6-methyl-3- [(2S,4S)-2-methyltetrahydropyran- 4-yl]hexahydropyrimidin-4-one hydrochloride | 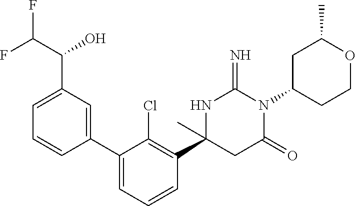 [SMILES: C[C@H]1C[C@@H](N2C(=N)N[C@](C)(c3cccc(-c4cccc([C@@H](O)C(F)F)c4)c3Cl)CC2=O)CCO1] | 0.75 | 492.4 |
| 26 | (6S)-6-(2-Chloro-3-{3-[(1S*)-2,2- difluoro-1-hydroxyethyl]phenyl}- phenyl)-2-imino-6-methyl-3- [(2S,4S)-2-methyltetrahydropyran- 4-yl]hexahydropyrimidin-4-one hydrochloride | 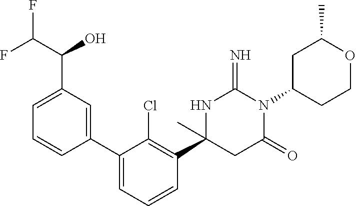 [SMILES: C[C@H]1C[C@@H](N2C(=N)N[C@](C)(c3cccc(-c4cccc([C@H](O)C(F)F)c4)c3Cl)CC2=O)CCO1] | 0.75 | 492.4 |
| 27 | (6S)-6-(2-Chloro-3-{3-[(1R*)- 2,2,2-trifluoro-1-hydroxyethyl]- phenyl}phenyl)-2-imino-6-methyl- 3-[(2S,4S)-2-methyltetrahydro- pyran-4-yl\|hexahydropyrimidin-4- one hydrochloride | 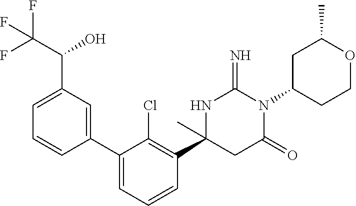 [SMILES: C[C@H]1C[C@@H](N2C(=N)N[C@](C)(c3cccc(-c4cccc([C@@H](O)C(F)(F)F)c4)c3Cl)CC2=O)CCO1] | 1.69 | 510.2 |
| 28 | (6S)-6-(2-Chloro-3-{3-[(1S*)-2,2,2- trifluoro-1-hydroxyethyl]phenyl}- phenyl)-2-imino-6-methyl-3- [(2S,4S)-2-methyltetrahydropyran- 4-yl]hexahydropyrimidin-4-one hydrochloride | 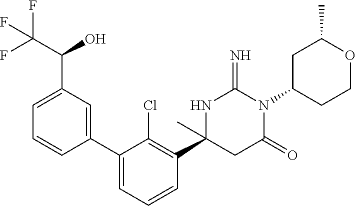 [SMILES: C[C@H]1C[C@@H](N2C(=N)N[C@](C)(c3cccc(-c4cccc([C@H](O)C(F)(F)F)c4)c3Cl)CC2=O)CCO1] | 1.69 | 510.2 |
| 29 | (6S)-6-{2-Chloro-3-[3-(morpholin- 4-yl)phenyl]phenyl}-2-imino-6- methyl-3-[(2S,4S)-2-methyl- tetrahydropyran-4-yl]hexahydro- pyrimidin-4-one trifluoroacetic acid salt | 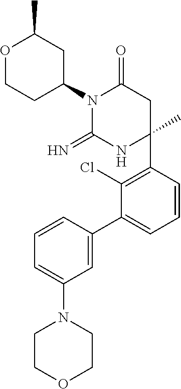 [SMILES: C[C@H]1C[C@@H](N2C(=N)N[C@](C)(c3cccc(-c4cccc(N5CCOCC5)c4)c3Cl)CC2=O)CCO1] | 0.83 | 497.3 |
| 30 | (6S)-6-[2-Chloro-3-(1-methyl- pyrrolo[2,3-b]pyridin-2-yl)phenyl]- 2-imino-6-methyl-3-[(2S,4S)-2- methyltetrahydropyran-4-yl]- hexahydropyrimidin-4-one trifluoroacetic acid salt | 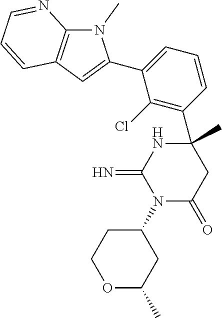 [SMILES: C[C@H]1C[C@@H](N2C(=N)N[C@](C)(c3cccc(-c4cc5cccnc5n4C)c3Cl)CC2=O)CCO1] | 0.78 | 466.2 |
| 31 | (6S)-6-[2-Chloro-3-(6-cyclopropyl- pyridin-3-yl)phenyl]-2-imino-6- methyl-3-[(2S,4S)-2-methyl- tetrahydropyran-4-yl]hexahydro- pyrimidin-4-one trifluoroacetic acid salt | 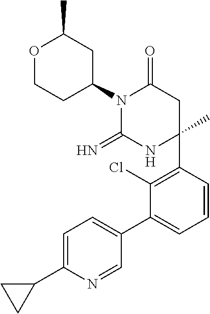 [SMILES: C[C@H]1C[C@@H](N2C(=N)N[C@](C)(c3cccc(-c4ccc(C5CC5)nc4)c3Cl)CC2=O)CCO1] | 0.71 | 453.2 |
| 32 | (6S)-6-[2-Chloro-3-(3-methyl- isoxazol-5-yl)phenyl]-2-imino-6- methyl-3-[(2S,4S)-2-methyl- tetrahydropyran-4-yl]hexahydro- pyrimidin-4-one trifluoroacetic acid salt | 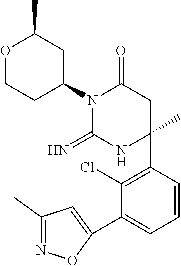 [SMILES: Cc1cc(-c2cccc([C@]3(C)CC(=O)N([C@H]4CCO[C@@H](C)C4)C(=N)N3)c2Cl)on1] | 0.67 | 417.2 |
| 33 | (6S)-6-[2-Chloro-3-(pyridazin-4- yl)phenyl]-2-imino-6-methyl-3- [(2S,4S)-2-methyltetrahydropyran- 4-yl]hexahydropyrimidin-4-one trifluoroacetic acid salt | 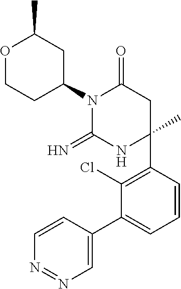 [SMILES: C[C@H]1C[C@@H](N2C(=N)N[C@](C)(c3cccc(-c4ccnnc4)c3Cl)CC2=O)CCO1] | 0.49 | 414.2 |
| 34 | (6S)-6-[2-Chloro-3-(1-methyl-2- oxopyridin-4-yl)phenyl]-2-imino-6- methyl-3-[(2S,4S)-2-methyl- tetrahydropyran-4-yl]hexahydro- pyrimidin-4-one trifluoroacetic acid salt | 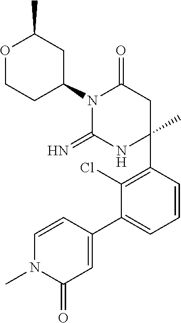 [SMILES: C[C@H]1C[C@@H](N2C(=N)N[C@](C)(c3cccc(-c4ccn(C)c(=O)c4)c3Cl)CC2=O)CCO1] | 0.54 | 443.2 |
| 35 | (6S)-6-[2-Chloro-3-(naphth-1-yl)- phenyl]-2-imino-6-methyl-3- [(2S,4S)-2-methyltetrahydropyran- 4-yl]hexahydropyrimidin-4-one trifluoroacetic acid salt | 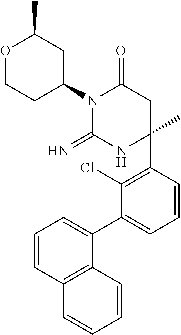 [SMILES: C[C@H]1C[C@@H](N2C(=N)N[C@](C)(c3cccc(-c4cccc5ccccc45)c3Cl)CC2=O)CCO1] | 0.97 | 462.3 |
| 36 | (6S)-6-[2-Chloro-3-(2-isopropyl- phenyl)phenyl]-2-imino-6-methyl- 3-[(2S,4S)-2-methyltetrahydro- pyran-4-yl]hexahydropyrimidin-4- one trifluoroacetic acid salt | 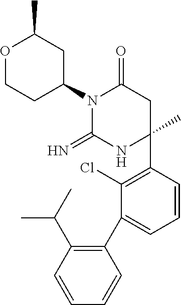 [SMILES: CC(C)c1ccccc1-c1cccc([C@]2(C)CC(=O)N([C@H]3CCO[C@@H](C)C3)C(=N)N2)c1Cl] | 1.01 | 454.3 |
| 37 | (6S)-6-{2-Chloro-3-[2-(trifluoro- methyl)phenyl]phenyl}-2-imino-6- methyl-3-[(2S,4S)-2-methyl- tetrahydropyran-4-yl]hexahydro- pyrimidin-4-one trifluoroacetic acid salt | 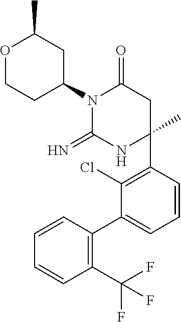 [SMILES: C[C@H]1C[C@@H](N2C(=N)N[C@](C)(c3cccc(-c4ccccc4C(F)(F)F)c3Cl)CC2=O)CCO1] | 0.93 | 480.2 |
| 38 | (6S)-6-[2-Chloro-3-(3,4-difluoro- phenyl)phenyl]-2-imino-6-methyl- 3-[(2S,4S)-2-methyltetrahydro- pyran-4-yl]hexahydropyrimidin-4- one trifluoroacetic acid salt | 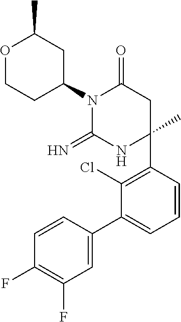 [SMILES: C[C@H]1C[C@@H](N2C(=N)N[C@](C)(c3cccc(-c4ccc(F)c(F)c4)c3Cl)CC2=O)CCO1] | 0.90 | 448.2 |
| 39 | (6S)-6-[2-Chloro-3-(pyridin-4-yl)- phenyl]-2-imino-6-methyl-3- [(2S,4S)-2-methyltetrahydropyran- 4-yl]hexahydropyrimidin-4-one trifluoroacetic acid salt | 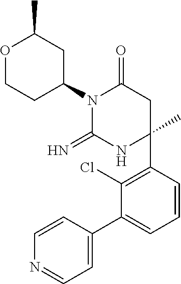 [SMILES: C[C@H]1C[C@@H](N2C(=N)N[C@](C)(c3cccc(-c4ccncc4)c3Cl)CC2=O)CCO1] | 0.49 | 413.0 |
| 40 | 3-(2-Chloro-3-{(4S)-2-imino-4- methyl-1-[(2S,4S)-2-methyl- tetrahydropyran-4-yl]-6-oxo- hexahydropyrimidin-4-yl}phenyl)- benzonitrile trifluoroacetic acid salt | 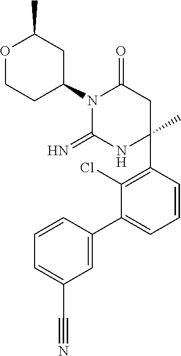 [SMILES: C[C@H]1C[C@@H](N2C(=N)N[C@](C)(c3cccc(-c4cccc(C#N)c4)c3Cl)CC2=O)CCO1] | 0.79 | 437.0 |
| 41 | (6S)-6-[2-Chloro-3-(pyrimidin-5- yl)phenyl]-2-imino-6-methyl-3- [(2S,4S)-2-methyltetrahydropyran- 4-yl]hexahydropyrimidin-4-one trifluoroacetic acid salt | 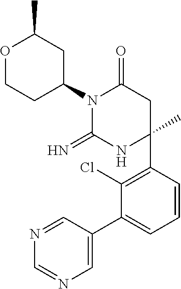 [SMILES: C[C@H]1C[C@@H](N2C(=N)N[C@](C)(c3cccc(-c4cncnc4)c3Cl)CC2=O)CCO1] | 0.53 | 414.2 |
| 42 | (6S)-6-[2-Chloro-3-(2,5-difluoro- phenyl)phenyl]-2-imino-6-methyl- 3-[(2S,4S)-2-methyltetrahydro- pyran-4-yl]hexahydropyrimidin-4- one trifluoroacetic acid salt | 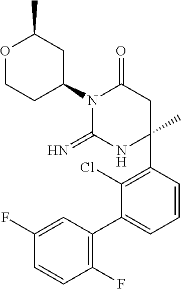 [SMILES: C[C@H]1C[C@@H](N2C(=N)N[C@](C)(c3cccc(-c4cc(F)ccc4F)c3Cl)CC2=O)CCO1] | 0.86 | 448.2 |
| 43 | (6S)-6-[2-Chloro-3-(6-methoxy- pyridin-3-yl)phenyl]-2-imino-6- methyl-3-[(2S,4S)-2-methyl- tetrahydropyran-4-yl]hexahydro- pyrimidin-4-one trifluoroacetic acid salt | 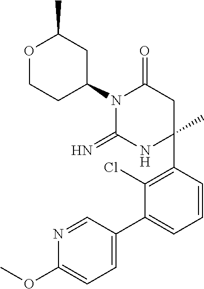 [SMILES: COc1ccc(-c2cccc([C@]3(C)CC(=O)N([C@H]4CCO[C@@H](C)C4)C(=N)N3)c2Cl)cn1] | 0.76 | 443.0 |
| 44 | (6S)-6-[2-Chloro-3-(3,5-difluoro- phenyl)phenyl]-2-imino-6-methyl- 3-[(2S,4S)-2-methyltetrahydro- pyran-4-yl]hexahydropyrimidin-4- one trifluoroacetic acid salt | 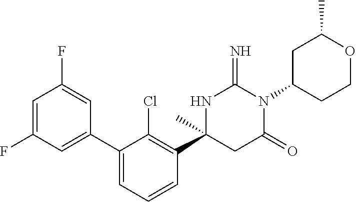 [SMILES: C[C@H]1C[C@@H](N2C(=N)N[C@](C)(c3cccc(-c4cc(F)cc(F)c4)c3Cl)CC2=O)CCO1] | 0.90 | 448.2 |
| 45 | (6S)-6-[2-Chloro-3-(1H-pyrazol-5- yl)phenyl]-2-imino-6-methyl-3- [(2S,4S)-2-methyltetrahydropyran- 4-yl]hexahydropyrimidin-4-one trifluoroacetic acid salt | 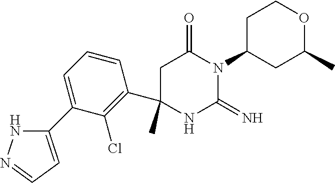 [SMILES: C[C@H]1C[C@@H](N2C(=N)N[C@](C)(c3cccc(-c4ccn[nH]4)c3Cl)CC2=O)CCO1] | 0.56 | 402.4 |
| 46 | (6S)-6-[2-Chloro-3-(2-chloro- phenyl)phenyl]-2-imino-6-methyl- 3-[(2S,4S)-2-methyltetrahydro- pyran-4-yl]hexahydropyrimidin-4- one trifluoroacetic acid salt | 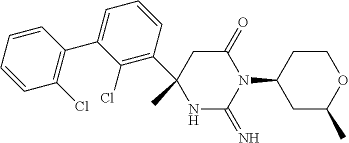 [SMILES: C[C@H]1C[C@@H](N2C(=N)N[C@](C)(c3cccc(-c4ccccc4Cl)c3Cl)CC2=O)CCO1] | 0.87 | 448.3 |
| 47 | (6S)-6-[2-Chloro-3-(2,6-difluoro- phenyl)phenyl]-2-imino-6-methyl- 3-[(2S,4S)-2-methyltetrahydro- pyran-4-yl]hexahydropyrimidin-4- one trifluoroacetic acid salt | 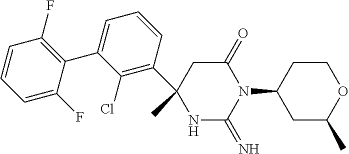 [SMILES: C[C@H]1C[C@@H](N2C(=N)N[C@](C)(c3cccc(-c4c(F)cccc4F)c3Cl)CC2=O)CCO1] | 0.83 | 448.3 |
| 48 | (6S)-6-{2-Chloro-3-[3-(trifluoro- methyl)phenyl]phenyl}-2-imino-6- methyl-3-[(2S,4S)-2-methyl- tetrahydropyran-4-yl]hexahydro- pyrimidin-4-one trifluoroacetic acid salt | 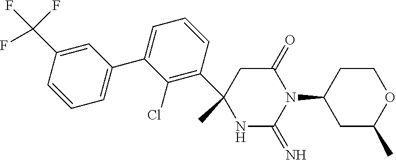 [SMILES: C[C@H]1C[C@@H](N2C(=N)N[C@](C)(c3cccc(-c4cccc(C(F)(F)F)c4)c3Cl)CC2=O)CCO1] | 0.96 | 480.4 |
| 49 | (6S)-6-[2-Chloro-3-(pyridin-3-yl)- phenyl]-2-imino-6-methyl-3- [(2S,4S)-2-methyltetrahydropyran- 4-yl]hexahydropyrimidin-4-one trifluoroacetic acid salt | 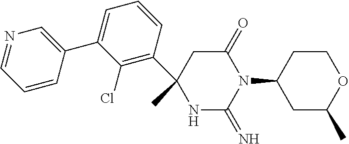 [SMILES: C[C@H]1C[C@@H](N2C(=N)N[C@](C)(c3cccc(-c4cccnc4)c3Cl)CC2=O)CCO1] | 0.55 | 413.3 |
| 50 | (6S)-6-[2-Chloro-3-(2,3-difluoro- phenyl)phenyl]-2-imino-6-methyl- 3-[(2S,4S)-2-methyltetrahydro- pyran-4-yl]hexahydropyrimidin-4- one trifluoroacetic acid salt | 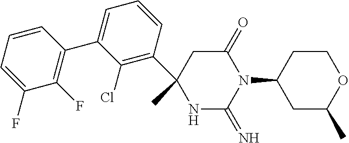 [SMILES: C[C@H]1C[C@@H](N2C(=N)N[C@](C)(c3cccc(-c4cccc(F)c4F)c3Cl)CC2=O)CCO1] | 0.85 | 448.4 |
| 51 | (6S)-6-[2-Chloro-3-(2,4-difluoro- phenyl)phenyl]-2-imino-6-methyl- 3-[(2S,4S)-2-methyltetrahydro- pyran-4-yl]hexahydropyrimidin-4- one trifluoroacetic acid salt | 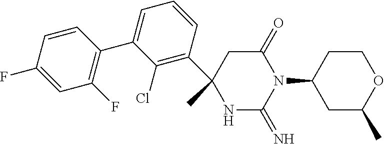 [SMILES: C[C@H]1C[C@@H](N2C(=N)N[C@](C)(c3cccc(-c4ccc(F)cc4F)c3Cl)CC2=O)CCO1] | 0.86 | 448.3 |
| 52 | (6S)-6-[2-Chloro-3-(2,5-dimethyl- pyrazol-3-yl)phenyl]-2-imino-6- methyl-3-[(2S,4S)-2-methyl- tetrahydropyran-4-yl]hexahydro- pyrimidin-4-one trifluoroacetic acid salt | 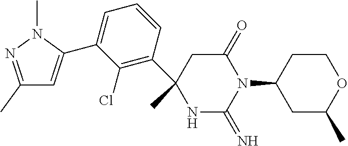 [SMILES: Cc1cc(-c2cccc([C@]3(C)CC(=O)N([C@H]4CCO[C@@H](C)C4)C(=N)N3)c2Cl)n(C)n1] | 0.66 | 430.4 |
| 53 | (6S)-6-{2-Chloro-3-[3-(1H-pyrazol- 5-yl)phenyl]phenyl}-2-imino-6- methyl-3-[(2S,4S)-2-methyl- tetrahydropyran-4-yl]hexahydro- pyrimidin-4-one trifluoroacetic acid salt | 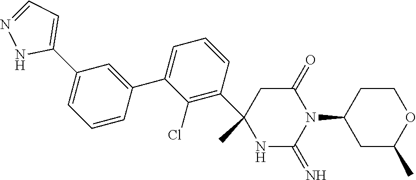 [SMILES: C[C@H]1C[C@@H](N2C(=N)N[C@](C)(c3cccc(-c4cccc(-c5ccn[nH]5)c4)c3Cl)CC2=O)CCO1] | 0.78 | 478.4 |
| 54 | (6S)-6-[2-Chloro-3-(isoxazol-4-yl)- phenyl]-2-imino-6-methyl-3- [(2S,4S)-2-methyltetrahydropyran- 4-yl]hexahydropyrimidin-4-one trifluoroacetic acid salt | 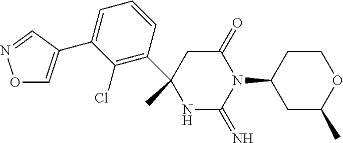 [SMILES: C[C@H]1C[C@@H](N2C(=N)N[C@](C)(c3cccc(-c4cnoc4)c3Cl)CC2=O)CCO1] | 0.61 | 403.4 |
| 55 | (6S)-6-[2-Chloro-3-(6-chloro- pyridin-3-yl)phenyl]-2-imino-6- methyl-3-[(2S,4S)-2-methyl- tetrahydropyran-4-yl]hexahydro- pyrimidin-4-one trifluoroacetic acid salt | 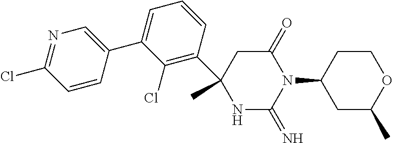 [SMILES: C[C@H]1C[C@@H](N2C(=N)N[C@](C)(c3cccc(-c4ccc(Cl)nc4)c3Cl)CC2=O)CCO1] | 0.75 | 447.3 |
| 56 | (6S)-6-[2-Chloro-3-(1-methyl- indazol-7-yl)phenyl]-2-imino-6- methyl-3-[(2S,4S)-2-methyl- tetrahydropyran-4-yl]hexahydro- pyrimidin-4-one trifluoroacetic acid salt | 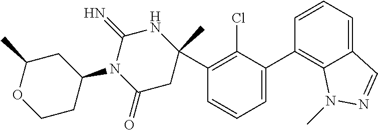 [SMILES: C[C@H]1C[C@@H](N2C(=N)N[C@](C)(c3cccc(-c4cccc5cnn(C)c45)c3Cl)CC2=O)CCO1] | 0.78 | 466.4 |
| 57 | (6S)-6-[2-Chloro-3-(3-chloro- pyridin-4-yl)phenyl]-2-imino-6- methyl-3-[(2S,4S)-2-methyl- tetrahydropyran-4-yl]hexahydro- pyrimidin-4-one trifluoroacetic acid salt | 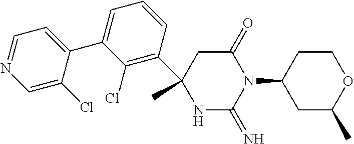 [SMILES: C[C@H]1C[C@@H](N2C(=N)N[C@](C)(c3cccc(-c4ccncc4Cl)c3Cl)CC2=O)CCO1] | 0.69 | 447.3 |
| 58 | (6S)-6-[2-Chloro-3-(5-methoxy- pyridin-3-yl)phenyl]-2-imino-6- methyl-3-[(2S,4S)-2-methyl- tetrahydropyran-4-yl]hexahydro- pyrimidin-4-one trifluoroacetic acid salt | 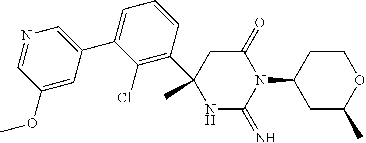 [SMILES: COc1cncc(-c2cccc([C@]3(C)CC(=O)N([C@H]4CCO[C@@H](C)C4)C(=N)N3)c2Cl)c1] | 0.64 | 443.3 |
| 59 | (6S)-6-[2-Chloro-3-(2-methoxy- pyridin-4-yl)phenyl]-2-imino-6- methyl-3-[(2S,4S)-2-methyl- tetrahydropyran-4-yl]hexahydro- pyrimidin-4-one trifluoroacetic acid salt | 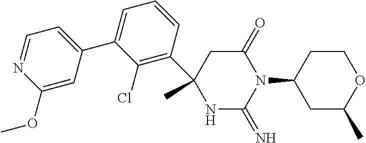 [SMILES: COc1cc(-c2cccc([C@]3(C)CC(=O)N([C@H]4CCO[C@@H](C)C4)C(=N)N3)c2Cl)ccn1] | 0.74 | 443.3 |
| 60 | 4-(2-Chloro-3-{(4S)-2-imino-4- methyl-1-[(2S,4S)-2-methyl- tetrahydropyran-4-yl]-6-oxo- hexahydropyrimidin-4-yl}phenyl)- benzonitrile trifluoroacetic acid salt | 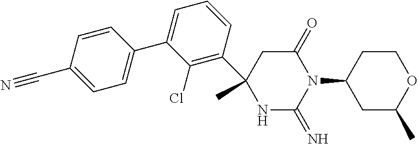 [SMILES: C[C@H]1C[C@@H](N2C(=N)N[C@](C)(c3cccc(-c4ccc(C#N)cc4)c3Cl)CC2=O)CCO1] | 0.78 | 437.3 |
| 61 | (6S)-6-[2-Chloro-3-(2-chloro- pyridin-3-yl)phenyl]-2-imino-6- methyl-3-[(2S,4S)-2-methyl- tetrahydropyran-4-yl]hexahydro- pyrimidin-4-one trifluoroacetic acid salt | 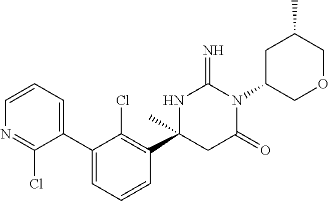 [SMILES: C[C@@H]1COC[C@H](N2C(=N)N[C@](C)(c3cccc(-c4cccnc4Cl)c3Cl)CC2=O)C1] | 0.69 | 447.4 |
| 62 | (6S)-6-[2-Chloro-3-(pyrazolo[1,5- a]pyrimidin-3-yl)phenyl]-2-imino- 6-methyl-3-[(2S,4S)-2-methyl- tetrahydropyran-4-yl]hexahydro- pyrimidin-4-one trifluoroacetic acid salt | 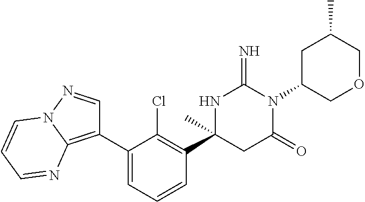 [SMILES: C[C@@H]1COC[C@H](N2C(=N)N[C@](C)(c3cccc(-c4cnn5cccnc45)c3Cl)CC2=O)C1] | 0.62 | 453.4 |
| 63 | (6S)-6-[2-Chloro-3-(2,4-difluoro- phenyl)phenyl]-2-imino-6-methyl- 3-[(2R*,4R*)-2-methyl-1,1-dioxo- thian-4-yl]hexahydropyrimidin-4- one hydrochloride | 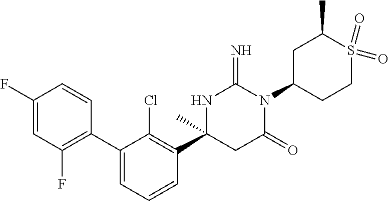 [SMILES: C[C@@H]1C[C@H](N2C(=N)N[C@](C)(c3cccc(-c4ccc(F)cc4F)c3Cl)CC2=O)CCS1(=O)=O] | 0.81 | 496.3 |
| 64 | (6S)-6-[2-Chloro-3-(2,4-difluoro- phenyl)phenyl]-2-imino-6-methyl- 3-[(2S*,4S*)-2-methyl-1,1-dioxo- thian-4-yl]hexahydropyrimidin-4- one hydrochloride | 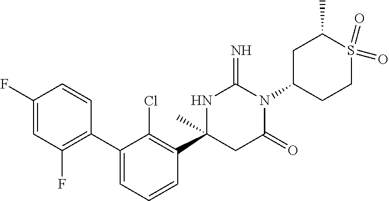 [SMILES: C[C@H]1C[C@@H](N2C(=N)N[C@](C)(c3cccc(-c4ccc(F)cc4F)c3Cl)CC2=O)CCS1(=O)=O] | 0.82 | 496.4 |
| 65 | (6S)-6-[2-Chloro-3-(2,4-difluoro- phenyl)phenyl]-2-imino-6-methyl- 3-[(2S*,4R*)-2-methyl-1,1-dioxo- thian-4-yl]hexahydropyrimidin-4- one hydrochloride | 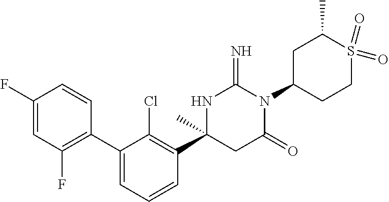 [SMILES: C[C@H]1C[C@H](N2C(=N)N[C@](C)(c3cccc(-c4ccc(F)cc4F)c3Cl)CC2=O)CCS1(=O)=O] | 0.81 | 496.4 |
| 66 | (6S)-6-[2-Chloro-3-(2,4-difluoro- phenyl)phenyl]-2-imino-6-methyl- 3-[(2R*,4S*)-2-methyl-1,1-dioxo- thian-4-yl]hexahydropyrimidin-4- one hydrochloride | 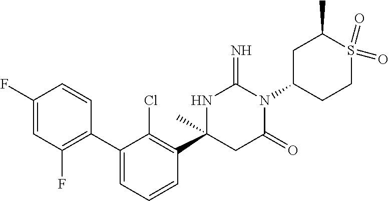 [SMILES: C[C@@H]1C[C@@H](N2C(=N)N[C@](C)(c3cccc(-c4ccc(F)cc4F)c3Cl)CC2=O)CCS1(=O)=O] | 0.79 | 496.4 |
| 67 | (6S)-6-[2-Chloro-3-(6-cyclopropyl- pyridin-3-yl)phenyl]-2-imino-6- methyl-3-[(2S*,4S*)-2-methyl-1,1- dioxothian-4-yl]hexahydro- pyrimidin-4-one hydrochloride | 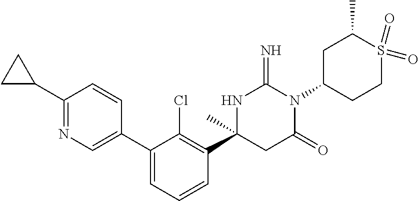 [SMILES: C[C@H]1C[C@@H](N2C(=N)N[C@](C)(c3cccc(-c4ccc(C5CC5)nc4)c3Cl)CC2=O)CCS1(=O)=O] | 0.65 | 501.4 |
| 68 | (6S)-6-[2-Chloro-3-(6-cyclopropyl- pyridin-3-yl)phenyl]-2-imino-6- methyl-3-[(2R*,4S*)-2-methyl-1,1- dioxothian-4-yl]hexahydro- pyrimidin-4-one hydrochloride | 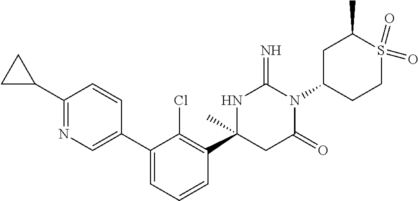 [SMILES: C[C@@H]1C[C@@H](N2C(=N)N[C@](C)(c3cccc(-c4ccc(C5CC5)nc4)c3Cl)CC2=O)CCS1(=O)=O] | 0.66 | 501.3 |
| 69 | (6S)-6-[2-Chloro-3-(6-cyclopropyl- pyridin-3-yl)phenyl]-2-imino-6- methyl-3-[(2S*,4R*)-2-methyl-1,1- dioxothian-4-yl]hexahydro- pyrimidin-4-one hydrochloride | 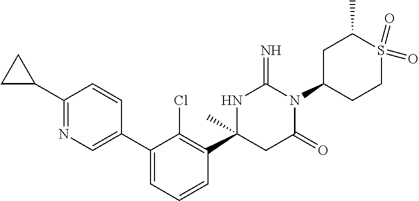 [SMILES: C[C@H]1C[C@H](N2C(=N)N[C@](C)(c3cccc(-c4ccc(C5CC5)nc4)c3Cl)CC2=O)CCS1(=O)=O] | 0.64 | 501.3 |
| 70 | (6S)-6-{2-Chloro-3-[6-(difluoro- methoxy)pyridin-3-yl]phenyl}-2- imino-6-methyl-3-[(2R*,4R*)-2- methyl-1,1-dioxothian-4-yl]- hexahydropyrimidin-4-one hydrochloride | 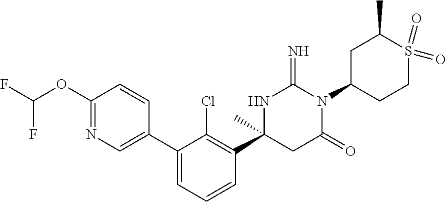 [SMILES: C[C@@H]1C[C@H](N2C(=N)N[C@](C)(c3cccc(-c4ccc(OC(F)F)nc4)c3Cl)CC2=O)CCS1(=O)=O] | 0.80 | 527.2 |
| 71 | (6S)-6-{2-Chloro-3-[6-(difluoro- methoxy)pyridin-3-yl]phenyl}-2- imino-6-methyl-3-[(2S*,4S*)-2- methyl-1,1-dioxothian-4-yl]- hexahydropyrimidin-4-one hydrochloride | 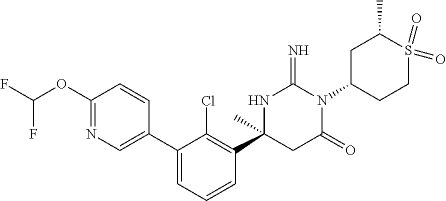 [SMILES: C[C@H]1C[C@@H](N2C(=N)N[C@](C)(c3cccc(-c4ccc(OC(F)F)nc4)c3Cl)CC2=O)CCS1(=O)=O] | 0.81 | 527.4 |
| 72 | (6S)-6-{2-Chloro-3-[6-(difluoro- methoxy)pyridin-3-yl\|phenyl}-2- imino-6-methyl-3-[(2R*,4S*)-2- methyl-1,1-dioxothian-4-yl]- hexahydropyrimidin-4-one hydrochloride | 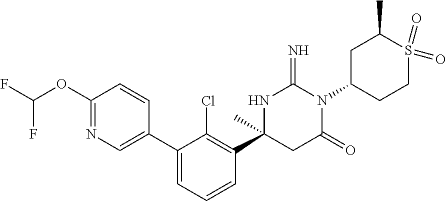 [SMILES: C[C@@H]1C[C@@H](N2C(=N)N[C@](C)(c3cccc(-c4ccc(OC(F)F)nc4)c3Cl)CC2=O)CCS1(=O)=O] | 0.80 | 527.3 |
| 73 | (6S)-6-{2-Chloro-3-[6-(difluoro- methoxy)pyridin-3-yl]phenyl}-2- imino-6-methyl-3-[(2S*,4R*)-2- methyl-1,1-dioxothian-4-yl]- hexahydropyrimidin-4-one hydrochloride | 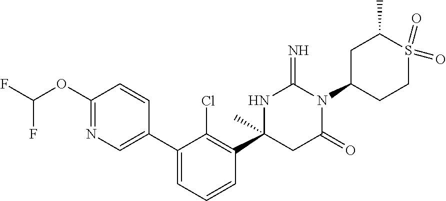 [SMILES: C[C@H]1C[C@H](N2C(=N)N[C@](C)(c3cccc(-c4ccc(OC(F)F)nc4)c3Cl)CC2=O)CCS1(=O)=O] | 0.80 | 527.3 |
| 74 | (6S)-6-[2-Chloro-3-(5-chloro- pyridin-2-yl)phenyl]-2-imino-6- methyl-3-[(2R*,4R*)-2-methyl-1,1- dioxothian-4-yl]hexahydro- pyrimidin-4-one hydrochloride | 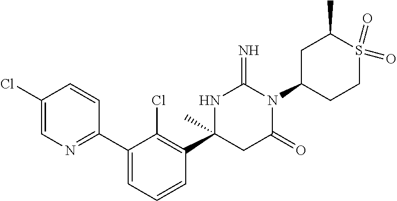 [SMILES: C[C@@H]1C[C@H](N2C(=N)N[C@](C)(c3cccc(-c4ccc(Cl)cn4)c3Cl)CC2=O)CCS1(=O)=O] | 0.74 | 497.2 |
| 75 | (6S)-6-[2-Chloro-3-(5-chloro- pyridin-2-yl)phenyl]-2-imino-6- methyl-3-[(2S*,4S*)-2-methyl-1,1- dioxothian-4-yl]hexahydro- pyrimidin-4-one hydrochloride | 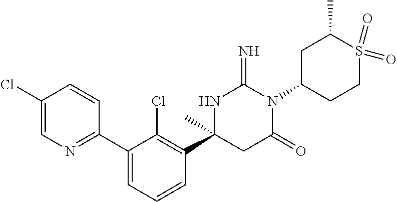 [SMILES: C[C@H]1C[C@@H](N2C(=N)N[C@](C)(c3cccc(-c4ccc(Cl)cn4)c3Cl)CC2=O)CCS1(=O)=O] | 0.71 | 495.3 |
| 76 | (6S)-6-[2-Chloro-3-(5-chloro- pyridin-2-yl)phenyl]-2-imino-6- methyl-3-[(2S*,4R*)-2-methyl-1,1- dioxothian-4-yl]hexahydro- pyrimidin-4-one hydrochloride | 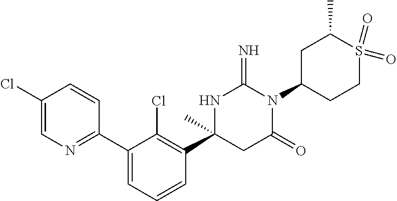 [SMILES: C[C@H]1C[C@H](N2C(=N)N[C@](C)(c3cccc(-c4ccc(Cl)cn4)c3Cl)CC2=O)CCS1(=O)=O] | 0.72 | 495.2 |
| 77 | (6S)-6-[2-Chloro-3-(5-chloro- pyridin-2-yl)phenyl]-2-imino-6- methyl-3-[(2R*,4S*)-2-methyl-1,1- dioxothian-4-yl]hexahydro- pyrimidin-4-one hydrochloride | 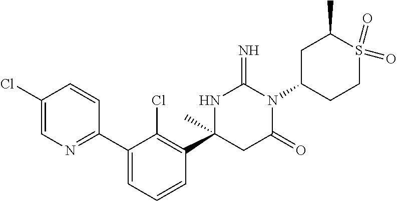 [SMILES: C[C@@H]1C[C@@H](N2C(=N)N[C@](C)(c3cccc(-c4ccc(Cl)cn4)c3Cl)CC2=O)CCS1(=O)=O] | 0.69 | 495.2 |
| 78 | (6S)-6-{2-Chloro-3-[6-(trifluoro- methoxy)pyridin-3-yl]phenyl}-2- imino-6-methyl-3-[(2S*,4S*)-2- methyl-1,1-dioxothian-4-yl]- hexahydropyrimidin-4-one hydrochloride | 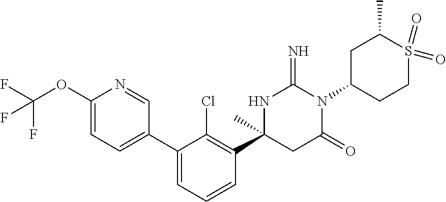 [SMILES: C[C@H]1C[C@@H](N2C(=N)N[C@](C)(c3cccc(-c4ccc(OC(F)(F)F)nc4)c3Cl)CC2=O)CCS1(=O)=O] | 0.86 | 545.3 |
| 79 | (6S)-6-{2-Chloro-3-[6-(trifluoro- methoxy)pyridin-3-yl]phenyl}-2- imino-6-methyl-3-[(2R*,4R*)-2- methyl-1,1-dioxothian-4-yl]- hexahydropyrimidin-4-one hydrochloride | 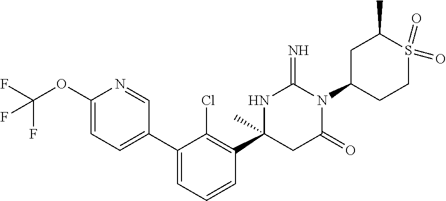 [SMILES: C[C@@H]1C[C@H](N2C(=N)N[C@](C)(c3cccc(-c4ccc(OC(F)(F)F)nc4)c3Cl)CC2=O)CCS1(=O)=O] | 0.84 | 545.3 |
| 80 | (6S)-6-{2-Chloro-3-[6-(trifluoro- methoxy)pyridin-3-yl]phenyl}-2- imino-6-methyl-3-[(2S*,4R*)-2- methyl-1,1-dioxothian-4-yl]- hexahydropyrimidin-4-one hydrochloride | 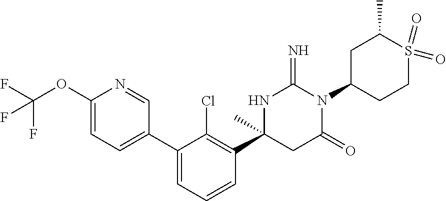 [SMILES: C[C@H]1C[C@H](N2C(=N)N[C@](C)(c3cccc(-c4ccc(OC(F)(F)F)nc4)c3Cl)CC2=O)CCS1(=O)=O] | 0.84 | 545.2 |
| 81 | (6S)-6-{2-Chloro-3-[6-(trifluoro- methoxy)pyridin-3-yl]phenyl}-2- imino-6-methyl-3-[(2R*,4S*)-2- methyl-1,1-dioxothian-4-yl]- hexahydropyrimidin-4-one hydrochloride | 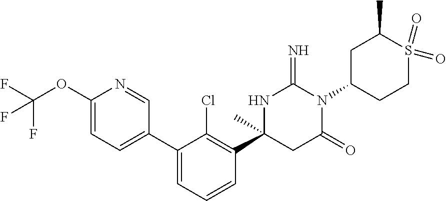 [SMILES: C[C@@H]1C[C@@H](N2C(=N)N[C@](C)(c3cccc(-c4ccc(OC(F)(F)F)nc4)c3Cl)CC2=O)CCS1(=O)=O] | 0.83 | 545.2 |
| 82 | (6S)-6-[2-Chloro-3-(5-chloro- pyridin-3-yl)phenyl]-3- [(1S*,3S*,4R*)-4-hydroxy-3- methylcyclohexyl]-2-imino-6- methylhexahydropyrimidin-4-one hydrochloride | 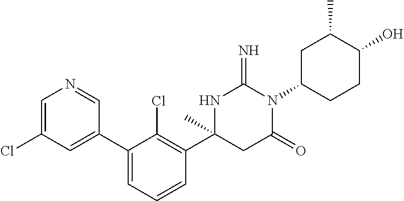 [SMILES: C[C@H]1C[C@@H](N2C(=N)N[C@](C)(c3cccc(-c4cncc(Cl)c4)c3Cl)CC2=O)CC[C@H]1O] | 0.74 | 461.4 |
| 83 | (6S)-6-[2-Chloro-3-(5-chloro- pyridin-3-yl)phenyl]-3- [(1R*,3R*,4S*)-4-hydroxy-3- methylcyclohexyl]-2-imino-6- methylhexahydropyrimidin-4-one hydrochloride | 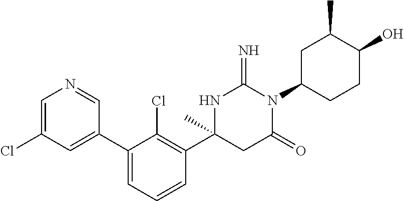 [SMILES: C[C@@H]1C[C@H](N2C(=N)N[C@](C)(c3cccc(-c4cncc(Cl)c4)c3Cl)CC2=O)CC[C@@H]1O] | 0.74 | 461.4 |
| 84 | (6S)-6-{2-Chloro-3-[1-(2,2,2- trifluoroethyl)pyrazol-4-yl]phenyl}- 3-[(1R*,3R*)-4,4-difluoro-3- hydroxycyclohexyl]-2-imino-6- methylhexahydropyrimidin-4-one hydrochloride | 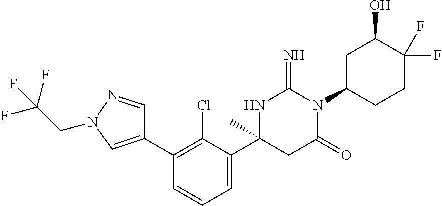 [SMILES: C[C@@]1(c2cccc(-c3cnn(CC(F)(F)F)c3)c2Cl)CC(=O)N([C@@H]2CCC(F)(F)[C@H](O)C2)C(=N)N1] | 0.64 | 520.3 |
| 85 | (6S)-6-{2-Chloro-3-[1-(2,2,2- trifluoroethyl)pyrazol-4-yl]phenyl}- 3-[(1S*,3S*)-4,4-difluoro-3- hydroxycyclohexyl]-2-imino-6- methylhexahydropyrimidin-4-one hydrochloride | 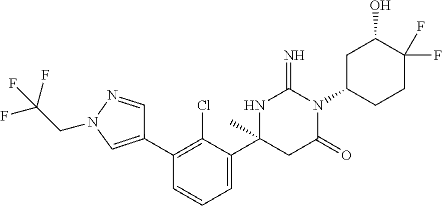 [SMILES: C[C@@]1(c2cccc(-c3cnn(CC(F)(F)F)c3)c2Cl)CC(=O)N([C@H]2CCC(F)(F)[C@@H](O)C2)C(=N)N1] | 0.70 | 520.3 |
| 86 | (6S)-6-[2-Chloro-3-(3-cyclopropyl- isoxazol-5-yl)phenyl]-3- [(1R*,3R*)-4,4-difluoro-3-hydroxy- cyclohexyl]-2-imino-6-methyl- hexahydropyrimidin-4-one hydrochloride | 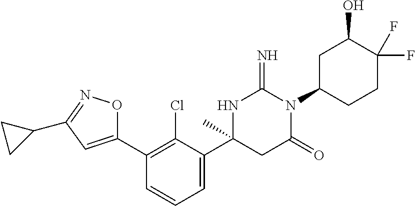 [SMILES: C[C@@]1(c2cccc(-c3cc(C4CC4)no3)c2Cl)CC(=O)N([C@@H]2CCC(F)(F)[C@H](O)C2)C(=N)N1] | 0.69 | 479.4 |
| 87 | (6S)-6-[2-Chloro-3-(3-cyclopropyl- isoxazol-5-yl)phenyl]-3-[(1S*,3S*)- 4,4-difluoro-3-hydroxycyclohexyl]- 2-imino-6-methylhexahydro- pyrimidin-4-one hydrochloride | 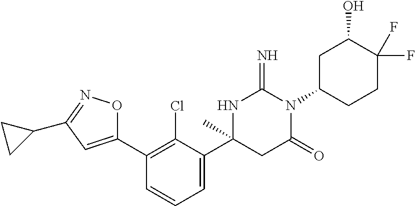 [SMILES: C[C@@]1(c2cccc(-c3cc(C4CC4)no3)c2Cl)CC(=O)N([C@H]2CCC(F)(F)[C@@H](O)C2)C(=N)N1] | 0.77 | 479.4 |
| 88 | (6S)-6-[2-Chloro-3-(2,4-difluoro- phenyl)phenyl]-3-[3-(dimethyl- amino)cyclobutyl]-2-imino-6- methylhexahydropyrimidin-4-one (isomer 1) | 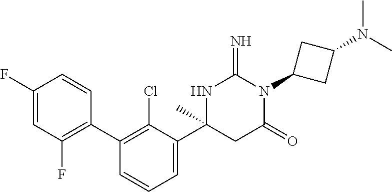 [SMILES: CN(C)[C@H]1C[C@H](N2C(=N)N[C@](C)(c3cccc(-c4ccc(F)cc4F)c3Cl)CC2=O)C1] | 0.45 | 447.3 |
| 89 | (6S)-6-[2-Chloro-3-(2,4-difluoro- phenyl)phenyl]-3-[3-(dimethyl- amino)cyclobutyl]-2-imino-6- methylhexahydropyrimidin-4-one (isomer 2) | 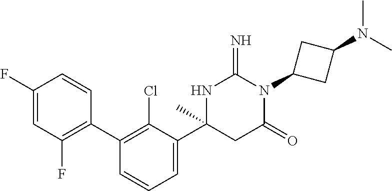 [SMILES: CN(C)[C@H]1C[C@@H](N2C(=N)N[C@](C)(c3cccc(-c4ccc(F)cc4F)c3Cl)CC2=O)C1] | 0.54 | 447.4 |
| 90 | (6S)-6-[2-Chloro-3-(3-chloro- phenyl)phenyl]-2-imino-6-methyl- 3-(1-methylpiperidin-4-yl)- hexahydropyrimidin-4-one | 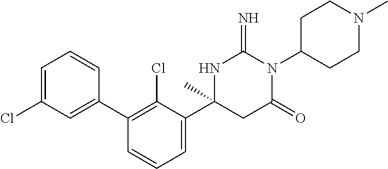 [SMILES: CN1CCC(N2C(=N)N[C@](C)(c3cccc(-c4cccc(Cl)c4)c3Cl)CC2=O)CC1] | 0.55 | 445.3 |
| 91 | (6S)-6-{2-Chloro-3-[2-chloro-4- (trifluoromethoxy)phenyl]phenyl}- 2-imino-6-methyl-3-(1-methyl- piperidin-4-yl)hexahydropyrimidin- 4-one | 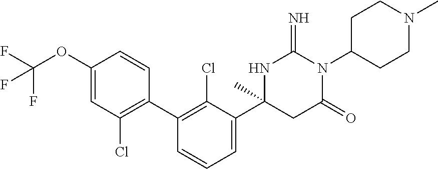 [SMILES: CN1CCC(N2C(=N)N[C@](C)(c3cccc(-c4ccc(OC(F)(F)F)cc4Cl)c3Cl)CC2=O)CC1] | 0.66 | 529.3 |
| 92 | (6S)-6-[2-Chloro-3-(2,5-dichloro- phenyl)phenyl]-2-imino-6-methyl- 3-(1-methylpiperidin-4-yl)- hexahydropyrimidin-4-one | 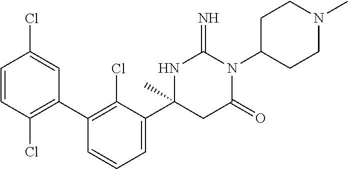 [SMILES: CN1CCC(N2C(=N)N[C@](C)(c3cccc(-c4cc(Cl)ccc4Cl)c3Cl)CC2=O)CC1] | 0.57 | 480.3 |
| 93 | (6S)-6-[2-Chloro-3-(2,4-difluoro- phenyl)phenyl]-2-imino-6-methyl- 3-(1-methylpiperidin-4-yl)- hexahydropyrimidin-4-one | 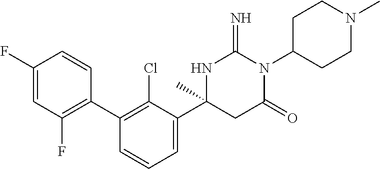 [SMILES: CN1CCC(N2C(=N)N[C@](C)(c3cccc(-c4ccc(F)cc4F)c3Cl)CC2=O)CC1] | 0.48 | 447.3 |
| 94 | (6S)-6-[2-Chloro-3-(3-chloro- phenyl)phenyl]-3-[(1R*)-1- cyclopropyl-2-hydroxyethyl]-2- imino-6-methylhexahydro- pyrimidin-4-one hydrochloride | 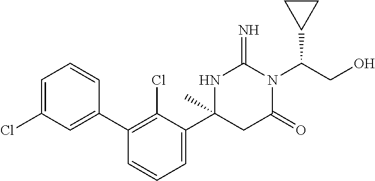 [SMILES: C[C@@]1(c2cccc(-c3cccc(Cl)c3)c2Cl)CC(=O)N([C@@H](CO)C2CC2)C(=N)N1] | 0.93 | 432.3 |
| 95 | (6S)-6-[2-Chloro-3-(3-chloro- phenyl)phenyl]-3-[(3R*,4S*)-3- hydroxytetrahydropyran-4-yl]-2- imino-6-methylhexahydro- pyrimidin-4-one hydrochloride | 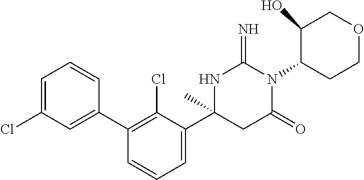 [SMILES: C[C@@]1(c2cccc(-c3cccc(Cl)c3)c2Cl)CC(=O)N([C@H]2CCOC[C@@H]2O)C(=N)N1] | 0.80 | 450.3 |
| 96 | (6S)-6-[2-Chloro-3-(3-chloro- phenyl)phenyl]-3-[(3S*,4R*)-3- hydroxytetrahydropyran-4-yl]-2- imino-6-methylhexahydro- pyrimidin-4-one hydrochloride | 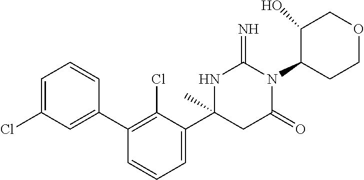 [SMILES: C[C@@]1(c2cccc(-c3cccc(Cl)c3)c2Cl)CC(=O)N([C@@H]2CCOC[C@H]2O)C(=N)N1] | 0.86 | 448.3 |
| 97 | (6S)-6-[2-Chloro-3-(2,4-difluoro- phenyl)phenyl]-3-[(1R*,3R*)-4,4- difluoro-3-hydroxycyclohexyl]-2- imino-6-methylhexahydro- pyrimidin-4-one hydrochloride | 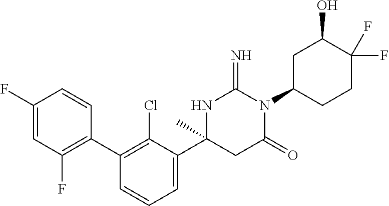 [SMILES: C[C@@]1(c2cccc(-c3ccc(F)cc3F)c2Cl)CC(=O)N([C@@H]2CCC(F)(F)[C@H](O)C2)C(=N)N1] | 0.79 | 484.3 |
| 98 | (6S)-6-[2-Chloro-3-(6-cyclopropyl- pyridin-3-yl)phenyl]-3-[(1R*,3R*)- 4,4-difluoro-3-hydroxycyclohexyl]- 2-imino-6-methylhexahydro- pyrimidin-4-one hydrochloride | 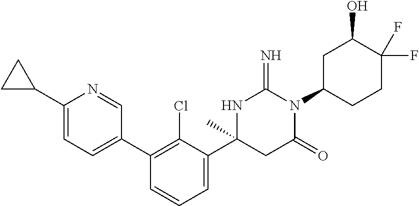 [SMILES: C[C@@]1(c2cccc(-c3ccc(C4CC4)nc3)c2Cl)CC(=O)N([C@@H]2CCC(F)(F)[C@H](O)C2)C(=N)N1] | 0.62 | 489.4 |
| 99 | (6S)-6-[2-Chloro-3-(6-cyclopropyl- pyridin-3-yl)phenyl]-3-[(1S*,3S*)- 4,4-difluoro-3-hydroxycyclohexyl]- 2-imino-6-methylhexahydro- pyrimidin-4-one hydrochloride | 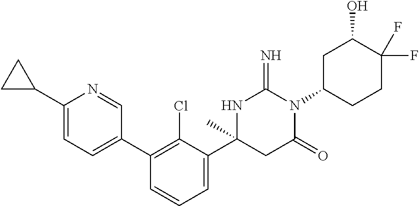 [SMILES: C[C@@]1(c2cccc(-c3ccc(C4CC4)nc3)c2Cl)CC(=O)N([C@H]2CCC(F)(F)[C@@H](O)C2)C(=N)N1] | 0.71 | 489.4 |
| 100 | (6S)-6-[2-Chloro-3-(3-chloro- phenyl)phenyl]-2-imino-6-methyl- 3-[(2S*,4S*)-2-methylpiperidin-4- yl]hexahydropyrimidin-4-one hydrochloride | 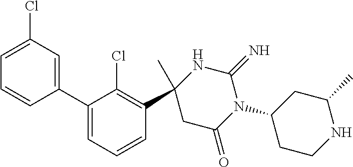 [SMILES: C[C@H]1C[C@@H](N2C(=N)N[C@](C)(c3cccc(-c4cccc(Cl)c4)c3Cl)CC2=O)CCN1] | 0.54 | 445.3 |
| 101 | (6S)-6-[2-Chloro-3-(3-chloro- phenyl)phenyl]-2-imino-6-methyl- 3-[(2R*,4R*)-2-methylpiperidin-4- yl]hexahydropyrimidin-4-one hydrochloride | 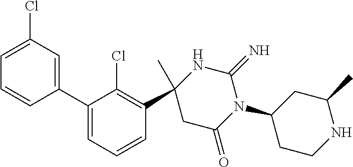 [SMILES: C[C@@H]1C[C@H](N2C(=N)N[C@](C)(c3cccc(-c4cccc(Cl)c4)c3Cl)CC2=O)CCN1] | 0.57 | 445.3 |
| 102 | (6S)-6-[2-Chloro-3-(2,4-difluoro- phenyl)phenyl]-2-imino-6-methyl- 3-[(2S*,4S*)-2-methylpiperidin-4- yl]hexahydropyrimidin-4-one hydrochloride | 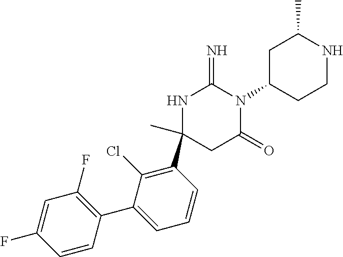 [SMILES: C[C@H]1C[C@@H](N2C(=N)N[C@](C)(c3cccc(-c4ccc(F)cc4F)c3Cl)CC2=O)CCN1] | 0.48 | 447.3 |
| 103 | (6S)-6-[2-Chloro-3-(2,4-difluoro- phenyl)phenyl]-2-imino-6-methyl- 3-[(2R*,4R*)-2-methylpiperidin-4- yl]hexahydropyrimidin-4-one hydrochloride | 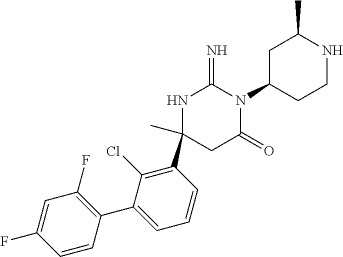 [SMILES: C[C@@H]1C[C@H](N2C(=N)N[C@](C)(c3cccc(-c4ccc(F)cc4F)c3Cl)CC2=O)CCN1] | 0.50 | 447.3 |
| 104 | (6S)-6-[2-Chloro-3-(3-chloro- phenyl)phenyl]-3-[(1S*,3S*,4S*)-4- hydroxy-3-methylcyclohexyl]-2- imino-6-methylhexahydro- pyrimidin-4-one hydrochloride | 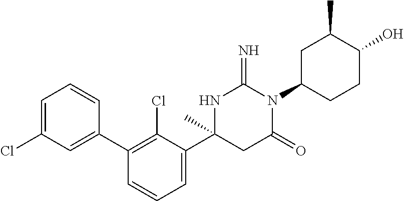 [SMILES: C[C@@H]1C[C@H](N2C(=N)N[C@](C)(c3cccc(-c4cccc(Cl)c4)c3Cl)CC2=O)CC[C@H]1O] | 0.86 | 460.4 |
| 105 | (6S)-6-[2-Chloro-3-(3-chloro- phenyl)phenyl]-3-[(1R*,3R*,4R*)- 4-hydroxy-3-methylcyclohexyl]-2- imino-6-methylhexahydro- pyrimidin-4-one hydrochloride | 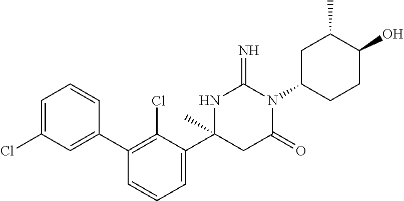 [SMILES: C[C@H]1C[C@@H](N2C(=N)N[C@](C)(c3cccc(-c4cccc(Cl)c4)c3Cl)CC2=O)CC[C@@H]1O] | 0.91 | 460.3 |
| 106 | (6S)-6-[2-Chloro-3-(2,4-difluoro- phenyl)phenyl]-3-[(2S*,4S*)-1,2- dimethylpiperidin-4-yl]-2-imino-6- methylhexahydropyrimidin-4-one hydrochloride | 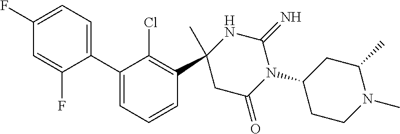 [SMILES: C[C@H]1C[C@@H](N2C(=N)N[C@](C)(c3cccc(-c4ccc(F)cc4F)c3Cl)CC2=O)CCN1C] | 0.44 | 461.4 |
| 107 | (6S)-6-[2-Chloro-3-(2,4-difluoro- phenyl)phenyl]-3-[(2R*,4R*)-1,2- dimethylpiperidin-4-yl]-2-imino-6- methylhexahydropyrimidin-4-one hydrochloride | 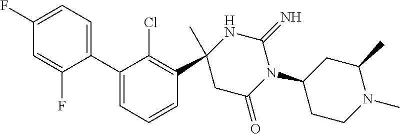 [SMILES: C[C@@H]1C[C@H](N2C(=N)N[C@](C)(c3cccc(-c4ccc(F)cc4F)c3Cl)CC2=O)CCN1C] | 0.51 | 461.3 |
| 108 | (6S)-6-{2-Chloro-3-[1-(3-fluoro- phenyl)-1,2,3-triazol-4-yl]phenyl}- 3-[(1S*,3S*)-4,4-difluoro-3- hydroxycyclohexyl]-2-imino-6- methylhexahydropyrimidin-4-one hydrochloride | 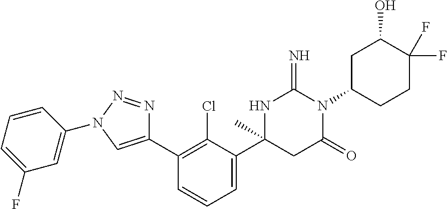 [SMILES: C[C@@]1(c2cccc(-c3cn(-c4cccc(F)c4)nn3)c2Cl)CC(=O)N([C@H]2CCC(F)(F)[C@@H](O)C2)C(=N)N1] | 0.77 | 533.4 |
| 109 | (6S)-6-{2-Chloro-3-[1-(2,4,5- trifluorophenyl)-1,2,3-triazol-4-yl]- phenyl}-2-imino-6-methyl-3- [(2S,4S)-2-methyltetrahydropyran- 4-yl]hexahydropyrimidin-4-one hydrochloride | 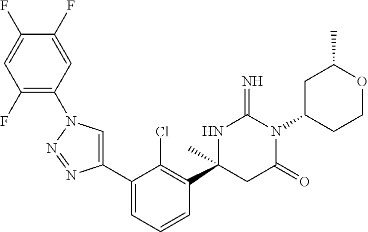 [SMILES: C[C@H]1C[C@@H](N2C(=N)N[C@](C)(c3cccc(-c4cn(-c5cc(F)c(F)cc5F)nn4)c3Cl)CC2=O)CCO1] | 0.86 | 553.3 |
| 110 | (6S)-6-{2-Chloro-3-[1-(2,2,2- trifluoroethyl)-1,2,3-triazol-4-yl]- phenyl}-2-imino-6-methyl-3- [(2S,4S)-2-methyltetrahydropyran- 4-yl]hexahydropyrimidin-4-one hydrochloride | 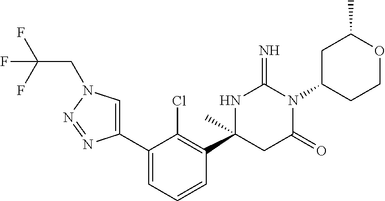 [SMILES: C[C@H]1C[C@@H](N2C(=N)N[C@](C)(c3cccc(-c4cn(CC(F)(F)F)nn4)c3Cl)CC2=O)CCO1] | 1.40 | 485.2 |
| 111 | (6S)-6-{2-Chloro-3-[1-(3-fluoro- phenyl)-1,2,3-triazol-4-yl]phenyl}- 2-imino-6-methyl-3-[(2S,4S)-2- methyltetrahydropyran-4-yl]- hexahydropyrimidin-4-one hydrochloride | 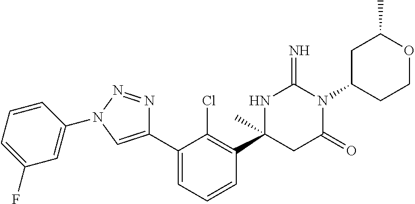 [SMILES: C[C@H]1C[C@@H](N2C(=N)N[C@](C)(c3cccc(-c4cn(-c5cccc(F)c5)nn4)c3Cl)CC2=O)CCO1] | 0.84 | 497.4 |
| 112 | (6S)-6-{2-Chloro-3-[1-(2,4,5- trifluorophenyl)-1,2,3-triazol-4-yl]- phenyl}-3-[(1S*,3S*,4R*)-4- hydroxy-3-methylcyclohexyl]-2- imino-6-methylhexahydro- pyrimidin-4-one hydrochloride | 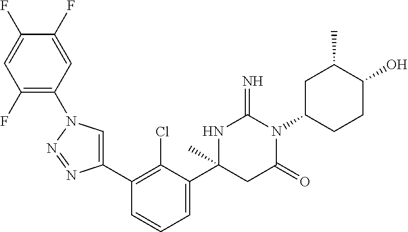 [SMILES: C[C@H]1C[C@@H](N2C(=N)N[C@](C)(c3cccc(-c4cn(-c5cc(F)c(F)cc5F)nn4)c3Cl)CC2=O)CC[C@H]1O] | 0.85 | 547.4 |
| 113 | (6S)-6-{2-Chloro-3-[1-(2,4,5- trifluorophenyl)-1,2,3-triazol-4-yl]- phenyl}-3-[(1R*,3R*,4S*)-4- hydroxy-3-methylcyclohexyl]-2- imino-6-methylhexahydro- pyrimidin-4-one hydrochloride | 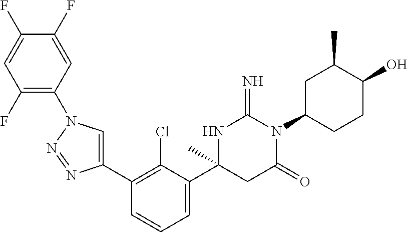 [SMILES: C[C@@H]1C[C@H](N2C(=N)N[C@](C)(c3cccc(-c4cn(-c5cc(F)c(F)cc5F)nn4)c3Cl)CC2=O)CC[C@@H]1O] | 0.85 | 547.4 |
| 114 | (6S)-6-(2-Chloro-3-{1-[2- (dimethylamino)ethyl]-1,2,3- triazol-4-yl}phenyl)-2-imino-6- methyl-3-[(2S,4S)-2-methyl- tetrahydropyran-4-yl]hexahydro- pyrimidin-4-one trifluoroacetic acid salt | 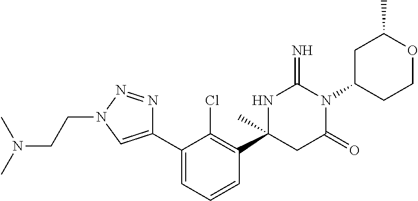 [SMILES: C[C@H]1C[C@@H](N2C(=N)N[C@](C)(c3cccc(-c4cn(CCN(C)C)nn4)c3Cl)CC2=O)CCO1] | 0.22 | 474.4 |
| 115 | (6S)-6-(2-Chloro-3-{1-[(1S)-1- (pyridin-3-yl)ethyl]-1,2,3-triazol-4- yl}phenyl)-2-imino-6-methyl-3- [(2S,4S)-2-methyltetrahydropyran- 4-yl]hexahydropyrimidin-4-one trifluoroacetic acid salt | 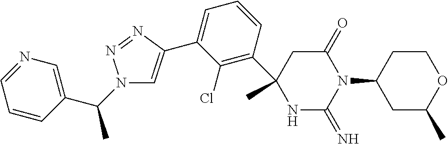 [SMILES: C[C@H]1C[C@@H](N2C(=N)N[C@](C)(c3cccc(-c4cn([C@@H](C)c5cccnc5)nn4)c3Cl)CC2=O)CCO1] | 0.59 | 508.4 |
| 116 | (6S)-6-(2-Chloro-3-{1-[(3,3- difluorocyclobutyl)methyl]-1,2,3- triazol-4-yl}phenyl)-2-imino-6- methyl-3-[(2S,4S)-2-methyl- tetrahydropyran-4-yl]hexahydro- pyrimidin-4-one trifluoroacetic acid salt | 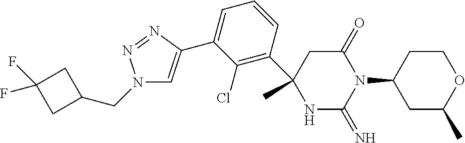 [SMILES: C[C@H]1C[C@@H](N2C(=N)N[C@](C)(c3cccc(-c4cn(CC5CC(F)(F)C5)nn4)c3Cl)CC2=O)CCO1] | 0.73 | 507.4 |
| 117 | (6S)-6-{2-Chloro-3-[1-(pyridin-3- ylmethyl)-1,2,3-triazol-4-yl]- phenyl}-2-imino-6-methyl-3- [(2S,4S)-2-methyltetrahydropyran- 4-yl]hexahydropyrimidin-4-one trifluoroacetic acid salt | 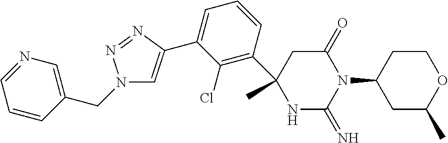 [SMILES: C[C@H]1C[C@@H](N2C(=N)N[C@](C)(c3cccc(-c4cn(Cc5cccnc5)nn4)c3Cl)CC2=O)CCO1] | 0.55 | 494.4 |
| 118 | (6S)-6-(2-Chloro-3-{1-[2- (pyrrolidin-1-yl)ethyl]-1,2,3- triazol-4-yl}phenyl)-2-imino-6- methyl-3-[(2S,4S)-2-methyl- tetrahydropyran-4-yl]hexahydro- pyrimidin-4-one trifluoroacetic acid salt | 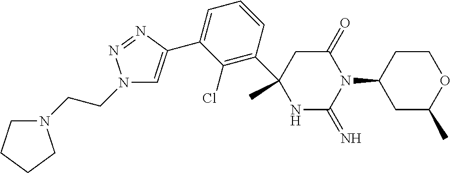 [SMILES: C[C@H]1C[C@@H](N2C(=N)N[C@](C)(c3cccc(-c4cn(CCN5CCCC5)nn4)c3Cl)CC2=O)CCO1] | 0.23 | 500.5 |
| 119 | (6S)-6-{2-Chloro-3-[1-(pyridin-4- yl)-1,2,3-triazol-4-yl]phenyl}-2- imino-6-methyl-3-[(2S,4S)-2- methyltetrahydropyran-4-yl]- hexahydropyrimidin-4-one trifluoroacetic acid salt | 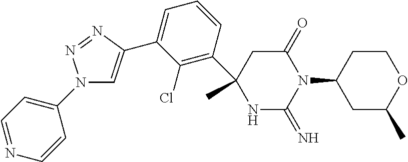 [SMILES: C[C@H]1C[C@@H](N2C(=N)N[C@](C)(c3cccc(-c4cn(-c5ccncc5)nn4)c3Cl)CC2=O)CCO1] | 0.63 | 480.4 |
| 120 | (6S)-6-{2-Chloro-3-[1-(pyridin-4- ylmethyl)-1,2,3-triazol-4-yl]- phenyl}-2-imino-6-methyl-3- [(2S,4S)-2-methyltetrahydropyran- 4-yl]hexahydropyrimidin-4-one trifluoroacetic acid salt | 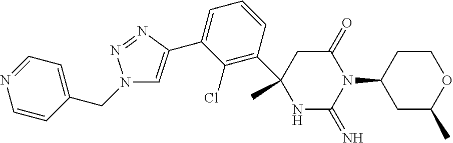 [SMILES: C[C@H]1C[C@@H](N2C(=N)N[C@](C)(c3cccc(-c4cn(Cc5ccncc5)nn4)c3Cl)CC2=O)CCO1] | 0.50 | 494.4 |
| 121 | (6S)-6-{2-Chloro-3-[1-(1-phenyl- vinyl)-1,2,3-triazol-4-yl]phenyl}-2- imino-6-methyl-3-[(2S,4S)-2- methyltetrahydropyran-4-yl]- hexahydropyrimidin-4-one trifluoroacetic acid salt | 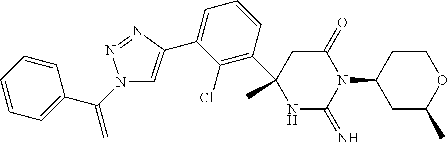 [SMILES: C=C(c1ccccc1)n1cc(-c2cccc([C@]3(C)CC(=O)N([C@H]4CCO[C@@H](C)C4)C(=N)N3)c2Cl)nn1] | 0.85 | 505.4 |
| 122 | (6S)-6-[2-Chloro-3-(1-cyclobutyl- 1,2,3-triazol-4-yl)phenyl]-2-imino- 6-methyl-3-[(2S,4S)-2-methyl- tetrahydropyran-4-yl]hexahydro- pyrimidin-4-one trifluoroacetic acid salt | 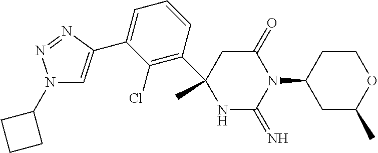 [SMILES: C[C@H]1C[C@@H](N2C(=N)N[C@](C)(c3cccc(-c4cn(C5CCC5)nn4)c3Cl)CC2=O)CCO1] | 0.71 | 457.4 |
| 123 | (6S)-6-{2-Chloro-3-[1-(2-methoxy- ethyl)-1,2,3-triazol-4-yl]phenyl}-2- imino-6-methyl-3-[(2S,4S)-2- methyltetrahydropyran-4-yl]- hexahydropyrimidin-4-one trifluoroacetic acid salt | 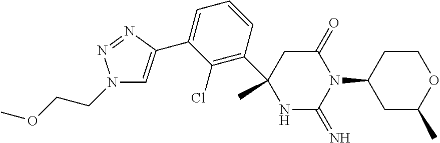 | 0.58 | 461.5 |
| 124 | (6S)-6-[3-(1-Benzyl-1,2,3-triazol-4- yl)-2-chlorophenyl]-2-imino-6- methyl-3-[(2S,4S)-2-methyl- tetrahydropyran-4-yl]hexahydro- pyrimidin-4-one trifluoroacetic acid salt | 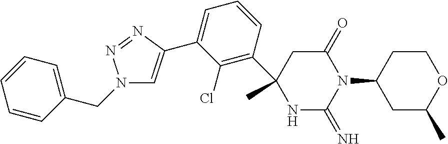 [SMILES: C[C@H]1C[C@@H](N2C(=N)N[C@](C)(c3cccc(-c4cn(Cc5ccccc5)nn4)c3Cl)CC2=O)CCO1] | 0.77 | 493.4 |
| 125 | (6S)-6-(2-Chloro-3-{1-[1-(2-oxa- bicyclo[2.1.1]hexan-1-yl)ethyl]- 1,2,3-triazol-4-yl}phenyl)-2-imino- 6-methyl-3-[(2S,4S)-2-methyl- tetrahydropyran-4-yl]hexahydro- pyrimidin-4-one trifluoroacetic acid salt | 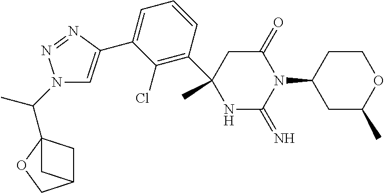 [SMILES: CC(n1cc(-c2cccc([C@]3(C)CC(=O)N([C@H]4CCO[C@@H](C)C4)C(=N)N3)c2Cl)nn1)C12CC(CO1)C2] | 0.70 | 513.4 |
| 126 | (6S)-6-[2-Chloro-3-(1-phenyl- 1,2,3-triazol-4-yl)phenyl]-2-imino- 6-methyl-3-[(2S,4S)-2-methyl- tetrahydropyran-4-yl]hexahydro- pyrimidin-4-one trifluoroacetic acid salt | 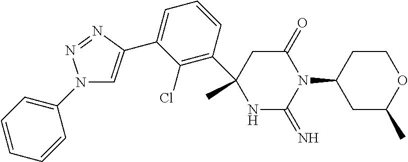 [SMILES: C[C@H]1C[C@@H](N2C(=N)N[C@](C)(c3cccc(-c4cn(-c5ccccc5)nn4)c3Cl)CC2=O)CCO1] | 0.80 | 479.4 |
| 127 | (6S)-6-[3-(1-Butyl-1,2,3-triazol-4- yl)-2-chlorophenyl]-2-imino-6- methyl-3-[(2S,4S)-2-methyl- tetrahydropyran-4-yl]hexahydro- pyrimidin-4-one trifluoroacetic acid salt | 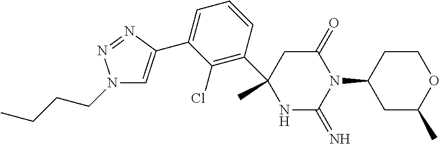 [SMILES: CCCCn1cc(-c2cccc([C@]3(C)CC(=O)N([C@H]4CCO[C@@H](C)C4)C(=N)N3)c2Cl)nn1] | 0.74 | 459.5 |
| 128 | (6S)-6-(2-Chloro-3-{1-[(1R)-1- (pyridin-3-yl)ethyl]-1,2,3-triazol-4- yl}phenyl)-2-imino-6-methyl-3- [(2S,4S)-2-methyltetrahydropyran- 4-yl]hexahydropyrimidin-4-one trifluoroacetic acid salt | 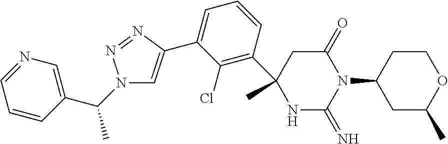 [SMILES: C[C@H](c1cccnc1)n1cc(-c2cccc([C@]3(C)CC(=O)N([C@H]4CCO[C@@H](C)C4)C(=N)N3)c2Cl)nn1] | 0.60 | 508.4 |
| 129 | (6S)-6-{3-[1-(Azepan-4-yl)-1,2,3- triazol-4-yl]-2-chlorophenyl}-2- imino-6-methyl-3-[(2S,4S)-2- methyltetrahydropyran-4-yl]- hexahydropyrimidin-4-one trifluoroacetic acid salt | 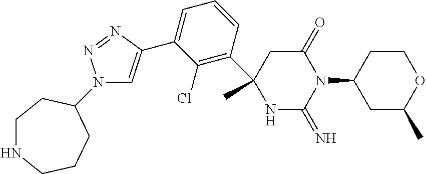 [SMILES: C[C@H]1C[C@@H](N2C(=N)N[C@](C)(c3cccc(-c4cn(C5CCCNCC5)nn4)c3Cl)CC2=O)CCO1] | 0.23 | 500.4 |

*Method 6: Examples 1, 3, 4, 6-26, 29-109 & 111-129
Method 4: Examples 2, 5, 27, 28 & 110

1. A compound of formula (I), or a pharmaceutically acceptable salt thereof: 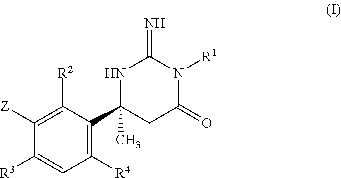
wherein
Z represents aryl or heteroaryl, either of which groups may be optionally substituted by one or more substituents;
R¹ represents C₃₋₇ cycloalkyl, C₃₋₇ cycloalkyl(C₁₋₆)alkyl, aryl(C₁₋₆)alkyl, C₃₋₇ heterocycloalkyl, C₃₋₇ heterocycloalkyl(C₁₋₆)alkyl, C₄₋₉ heterobicycloalkyl, C₄₋₉ spiroheterocycloalkyl or heteroaryl(C₁₋₆)alkyl, any of which groups may be optionally substituted by one or more substituents; and
R², R³ and R⁴ independently represent hydrogen, halogen or trifluoromethyl.

2. A compound as claimed in claim 1 wherein R¹ represents C₃₋₇ cycloalkyl, C₃₋₇ cycloalkyl(C₁₋₆)alkyl or C₃₋₇ heterocycloalkyl, any of which groups may be optionally substituted by one, two or three substituents independently selected from halogen, C₁₋₆ alkyl, hydroxy, oxo, C₁₋₆ alkoxy and di(C₁₋₆)alkylamino.

3. A compound as claimed in claim 1 or claim 2 wherein R³ represents hydrogen.

4. A compound as claimed in any one of the preceding claims wherein R⁴ represents hydrogen.

5. A compound as claimed in claim 1 represented by formula (IIA), or a pharmaceutically acceptable salt thereof: 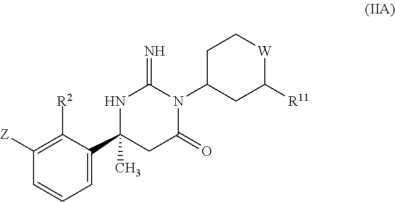
wherein
W represents O, S(O)₂, N—R¹², CH(OH) or CF₂;
R¹¹ represents hydrogen, methyl, hydroxy or methoxy;
R¹² represents hydrogen or methyl; and
Z and R² are as defined in claim 1.

6. A compound as claimed in any one of the preceding claims wherein Z represents phenyl, naphthyl, pyrrolo[2,3-b]pyridinyl, pyrazolyl, pyrazolo[1,5-a]-pyrimidinyl, indazolyl, isoxazolyl, benzimidazolyl, triazolyl, pyridinyl, pyridazinyl or pyrimidinyl, any of which groups may be optionally substituted by one, two or three substituents independently selected from halogen, cyano, C₁₋₆ alkyl, trifluoromethyl, trifluoroethyl, cyclopropyl, cyclobutyl, difluorocyclobutyl(C₁₋₆)alkyl, phenyl, fluoro-phenyl, trifluorophenyl, benzyl, phenyl(C₂₋₆)alkenyl, morpholinyl, azepanyl, pyrrolidinyl-(C₁₋₆)alkyl, 2-oxabicyclo[2.1.1]hexanyl(C₁₋₆)alkyl, pyrazolyl, pyridinyl, pyridinyl-(C₁₋₆)alkyl, (difluoro)(hydroxy)(C₁₋₆)alkyl, (trifluoro)(hydroxy)(C₁₋₆)alkyl, oxo, C₁₋₆ alkoxy, difluoromethoxy, trifluoromethoxy, C₁₋₆ alkoxy(C₁₋₆)alkyl, C₁₋₆ alkylsulfonyl, di(C₁₋₆)alkylamino(C₁₋₆)alkyl, C₁₋₆ alkylsulfonylamino, di(C₁₋₆)alkylsulfoximino and di(C₁₋₆)alkylphosphoryl.

7. A compound as claimed in any one of the preceding claims wherein R² represents chloro.

8. A compound as claimed in claim 1 as herein specifically disclosed in any one of the Examples.

9. A compound of formula (I) as defined in claim 1, or a pharmaceutically acceptable salt thereof, for use in therapy.

10. A compound of formula (I) as defined in claim 1, or a pharmaceutically acceptable salt thereof, for use in the treatment and/or prevention of malaria.

11. A pharmaceutical composition comprising a compound of formula (I) as defined in claim 1, or a pharmaceutically acceptable salt thereof, in association with a pharmaceutically acceptable carrier.

12. The use of a compound of formula (I) as defined in claim 1, or a pharmaceutically acceptable salt thereof, for the manufacture of a medicament for the treatment and/or prevention of malaria.

13. A method for the treatment and/or prevention of malaria, which comprises administering to a patient in need of such treatment an effective amount of a compound of formula (I) as defined in claim 1, or a pharmaceutically acceptable salt thereof.
